SEQUENCE LISTING (28 sequences)

SEQ ID NO: 1 - DNA, 20 bp, Ungulate erythroparvovirus 1
catcggctataaaaccccat

SEQ ID NO: 2 - DNA, 25 bp, Ungulate erythroparvovirus 1
ggacagtattatttccatgctctct

SEQ ID NO: 3 - DNA, 18 bp, Ungulate erythroparvovirus 1
caacgccatcaatcgcca

SEQ ID NO: 4 - DNA, 20 bp, Ungulate erythroparvovirus 1
gggttagaaacgctgcaatc

SEQ ID NO: 5 - DNA, 21 bp, Ungulate erythroparvovirus 1
agcaagcgagaggacatagtc

SEQ ID NO: 6 - DNA, 14 bp, Ungulate erythroparvovirus 1
ttaggtatggcgtt

SEQ ID NO: 7 - DNA, 22 bp, Ungulate erythroparvovirus 1
gcataaatgtgtcttggtgtgg

SEQ ID NO: 8 - DNA, 22 bp, Ungulate erythroparvovirus 1
attaatacgggagtgggagaca

SEQ ID NO: 9 - DNA, 15 bp, Ungulate erythroparvovirus 1
attgttgaggctgta

SEQ ID NO: 10 - DNA, 22 bp, Ungulate erythroparvovirus 1
tgtctcccactcccgtattaat

SEQ ID NO: 11 - DNA, 24 bp, Ungulate erythroparvovirus 1
aatgaccatcctctcacttaaagc

SEQ ID NO: 12 - DNA, 16 bp, Ungulate erythroparvovirus 1
atggaaacattgttac

SEQ ID NO: 13 - DNA, 28 bp, Ungulate erythroparvovirus 1
ttgttgattggctaaactatgtaaagtc

SEQ ID NO: 14 - DNA, 19 bp, Ungulate erythroparvovirus 1
gggctttccgctttatctc

SEQ ID NO: 15 - DNA, 14 bp, Ungulate erythroparvovirus 1
gctgataccgttca

SEQ ID NO: 16 - DNA, 23 bp, Ungulate erythroparvovirus 1
aacttggtcagagatcgaggagg

SEQ ID NO: 17 - DNA, 18 bp, Ungulate erythroparvovirus 1
ccgccagtgtgctggaat

SEQ ID NO: 18 - DNA, 15 bp, Ungulate erythroparvovirus 1
cgcccttcgaagcac

SEQ ID NO: 19 - DNA, 599 bp, Ungulate erythroparvovirus 1
aatgaaaattttcccttcaatgatgcgccgcataaatgtgtcttggtgtgggacgaaggccgaattacggccaaaattgttgaggctgtaaagagcattctggggggtcaggcggtgcgggtggatcagaaatgcaagggctctgtaagcttgtctcccactcccgtattaatcacatctaatgctgatattcgatatgtgcgtgatggaaacattgttactggggatcatgtaaaagctttaagtgagaggatggtcattgtacatttctctactccgtgccccgccaattttgggcttttgaaagcggaggaaattgttgattggctaaactatgtaaagtcatgcccggggagtatcactgctgataccgttcaggccacgtggggaacacgctccgcccccaatttatttgagataaagcggaaagccccacagcccgccagtcccattgaacctcagacggaggaacaagaagaagcagccgcatatcgctgtcagagcccaacttggtcagagatcgaggaggatttgagagcgtgcttcgaagggcgaattccagcacactggcggccgttactgcgcgcacattcaccaag

SEQ ID NO: 20 - DNA, 17 bp, Ungulate erythroparvovirus 1
atgcctccgagcaacac

SEQ ID NO: 21 - DNA, 26 bp, Ungulate erythroparvovirus 1
cctctctagactcttctctagattca

SEQ ID NO: 22 - DNA, 29 bp, Ungulate erythroparvovirus 1
cccagatatcacatcttctgtcggaaaca

SEQ ID NO: 23 - DNA, 18 bp, Ungulate erythroparvovirus 1
cggtggccacagcatatc

SEQ ID NO: 24 - DNA, 22 bp, Ungulate erythroparvovirus 1
gggacccactaggatacatttg

SEQ ID NO: 25 - DNA, 24 bp, Ungulate erythroparvovirus 1
tcagttcttgcgggactacttggc

SEQ ID NO: 26 - DNA, 19 bp, Ungulate erythroparvovirus 1
agcatatcctcggcctgat

SEQ ID NO: 27 - DNA, 19 bp, Ungulate erythroparvovirus 1
gataccgggacccactagg

SEQ ID NO: 28 - DNA, 23 bp, Ungulate erythroparvovirus 1
agttcttgcgggactacttggcc

(19) United States
(12) Patent Application Publication
(10) Pub. No.: US 2024/0026472 A1
(43) Pub. Date: Jan. 25, 2024

(54) REAL TIME PCR METHOD TO DETECT BOVINE PARVOVIRUS 3

(71) Applicant: AMGEN INC., Thousand Oaks, CA (US)

(72) Inventor: Samad AMINI BAVIL OLYAEE, Los Angeles, CA (US)

(73) Assignee: AMGEN INC., Thousand Oaks, CA (US)

(21) Appl. No.: 18/037,128

(22) PCT Filed: Dec. 16, 2021
(86) PCT No.: PCT/US2021/063668

§ 371 Date: May 16, 2023

Related U.S. Application Data
(60) Provisional application No. 63/126,939, filed on Dec. 17, 2020.
(60) Provisional application No. 63/211,607, filed on Jun. 17, 2021.

(51) Int. Cl. C12Q 1/70

(52) U.S. Cl. CPC C12Q 1/701; C12Q 2600/166

(57) ABSTRACT

The invention provides primer probe combinations for detecting DNA encoding Bovine parvovirus 3 (BPV-3) genomic DNA in the extracted DNA of a test sample. An internal positive control is also provided.

This application claims the benefit of U.S. Provisional Application No. 63/126,939, filed on Dec. 17, 2020, and U.S. Provisional Application No. 63/211,607, filed on Jun. 17, 2021 which are hereby incorporated by reference in its entirety and for all purposes as if fully set forth herein

The present application is being filed along with a Sequence Listing in electronic format. The Sequence Listing is provided as a file entitled A-2741-WO-PCT_ST25.txt, created Dec. 9, 2021, which is 6 KB in size. The information in the electronic format of the Sequence Listing is incorporated herein by reference in its entirety.

### FIELD OF DISCLOSURE

The present invention relates to the field of biopharmaceutical manufacturing. In particular, the invention provides a PCR assay for the qualitative or quantitative detection of Bovine parvovirus 3 (BPV-3) genomic DNA contamination in the extracted DNA of a test sample with an optional internal control.

### BACKGROUND

Manufacturing therapeutic biological drugs using cell culture processes carries an inherent risk of transmitting viral contaminants. Such contaminants can come from many sources, including materials and equipment, the use of reagents of animal origin during manufacture, and through contamination of the manufacturing system due to failures in the GMP process. Animal-derived raw materials, such as bovine serum (FBS), are sometimes used as a component of cell culture-based manufacturing processes and are one of the main sources for bovine-derived viral contamination in a biomanufacturing process. To meet the requirements of regulatory health to ensure absence of adventitious agents in biotherapeutics derived from mammalian cells, biomanufacturers need to take steps to detect, remove, and/or inactivate viral contamination. Raw materials can be tested for viral contamination prior to use and remedial action taken. Depending on the biologic molecule being manufactured, dedicated virus inactivation and removal steps can be added to the downstream purification process to ensure viral safety of biotherapeutics.

The Parvoviridae family includes single stranded DNA (ssDNA) viruses with small, non-enveloped capsids with T=1 icosahedral symmetry, known collectively as parvoviruses. Their diameter ranges between 18-26 nm. The viral capsid encompasses a ˜5 kb genome that encodes two main proteins, a non-structural (NS) protein, and a structural capsid (VP) protein. Virus in the Parvoviridae family infect a wide range of hosts and are divided into two subfamilies: the Parvovirinae and the Densovirinae, which infect vertebrate and arthropod hosts, respectively.

Bovine parvovirus 3 (BPV-3) belongs to the subfamily Parvovirinae, genus of Erythroparvovirus (species of Ungulate erythroparvovirus 1). While it is known that BPV-3 infect bovines, its pathogenesis and clinical manifestation remains unclear.

Per the requirements for testing in the Code of Federal Regulations (9 CFR113), the presence or absence of certain viruses in ingredients of animal origin used in production of biologics are required to be evaluated by incubating the animal-derived raw materials on specific indicator cells and which are subsequently observed for virus induced cytopathic effects and tested by hemadsorption or antibody fluorescence. Among the bovine parvoviruses, BPV-1 is capable of replicating in at least one of the selected indicator cell lines and can be detected by 9 CFR 113 test.

However, a permissive cell line to support BPV-3 replication has not been identified to date and, therefore, it cannot be detected by routine 9 CFR 113 testing. Although BPV-3 was detected by next-generation sequencing (NGS) a few years ago, it remains an emerging virus with limited information available. Traditional cell-culture based virus testing is not capable of supporting replication for BPV-3, thus, molecular biology assays are an essential alternative method to detect BPV-3 genomic DNA. The impact of BPV-3 in cell culture-based manufacturing remains unclear. There is a need for a rapid and specific method to detect the presence or absence of BPV-3 genomic DNA in raw materials such as fetal bovine serum (FBS) and/or unprocessed non-GMP bulk harvest samples containing FBS from cell culture-based manufacturing. The invention described herein meets this need.

### BRIEF SUMMARY OF THE INVENTION

The invention provides a composition comprising oligonucleotides selected from the groups consisting of a) oligonucleotides having the nucleic acid sequence of SEQ ID NO:1, SEQ ID NO:2, and SEQ ID NO:3; b) oligonucleotides having the nucleic acid sequence of SEQ ID NO:4, SEQ ID NO:5, and SEQ ID NO:6; c) oligonucleotides having the nucleic acid sequence of SEQ ID NO:7, SEQ ID NO:8, and SEQ ID NO:9; d) oligonucleotides having the nucleic acid sequence of SEQ ID NO:10, SEQ ID NO:11, and SEQ ID NO:12; and e) oligonucleotides having the nucleic acid sequence of SEQ ID NO:13, SEQ ID NO:14, SEQ ID NO:15; f) oligonucleotides having the nucleic acid sequence of SEQ ID NO:20, SEQ ID NO:21, and SEQ ID NO:22; g) oligonucleotides having the nucleic acid sequence of SEQ ID NO:23, SEQ ID NO:24, and SEQ ID NO:25; and h) oligonucleotides having the nucleic acid sequence of SEQ ID NO:26, SEQ ID NO:27, and SEQ ID NO:28. In one embodiment the composition optionally includes a second composition comprising oligonucleotides having the sequence of SEQ ID NO: 16, SEQ ID NO:17, and SEQ ID NO:18. In one embodiment the composition comprises oligonucleotides having the nucleic acid sequence of SEQ ID NO:7, SEQ ID NO:8, and SEQ ID NO:9. In one embodiment the composition comprises a first composition comprises oligonucleotides having the nucleic acid sequence of SEQ ID NO:7, SEQ ID NO:8, and SEQ ID NO:9 and a second composition comprises oligonucleotides having the sequence of SEQ ID NO: 16, SEQ ID NO:17, and SEQ ID NO:18.

The invention provides a reagent for detecting DNA encoding Bovine parvovirus 3 (BPV-3) genomic DNA in the extracted DNA of a test sample selected from the group consisting of a) a primer probe combination having the nucleic acid sequences of SEQ ID NO:1, SEQ ID NO:2, and SEQ ID NO:3; b) a primer probe combination having the nucleic acid sequences of SEQ ID NO:4, SEQ ID NO:5, and SEQ ID NO:6; c) a primer probe combination having the nucleic acid sequences of SEQ ID NO:7, SEQ ID NO:8, and SEQ ID NO:9; d) a primer probe combination having the nucleic acid sequences of SEQ ID NO:10, SEQ ID NO:11, and SEQ ID NO:12; and e) a primer probe combination having the nucleic acid sequences of SEQ ID NO:13, SEQ ID NO:14, SEQ ID NO:15; f) a primer probe combination having the nucleic acid sequences of SEQ ID NO:20, SEQ ID NO:21, and SEQ ID NO:22; g) a primer probe combination having the nucleic acid sequences of SEQ ID NO:23, SEQ ID NO:24, and SEQ ID NO:25; and h) a primer probe combination having the nucleic acid sequences of SEQ ID NO:26, SEQ ID NO:27, and SEQ ID NO:28.

In one embodiment SEQ ID NO: 3, SEQ ID NO:6, SEQ ID NO:9, SEQ ID NO:12, SEQ ID NO:15, SEQ ID NO:22, SEQ ID NO:25; or SEQ ID NO:28 have a fluorescent reporter dye and/or a non-fluorescent quencher. In a related embodiment one or more of SEQ ID NO: 3, SEQ ID NO:6, SEQ ID NO:9, SEQ ID NO:12, SEQ ID NO:15, SEQ ID NO:22, SEQ ID NO:25; or SEQ ID NO:28 have a fluorescent reporter dye 6-carboxyfluorescein (FAM) at the 5′ end and/or either a Minor Groove Binder non-fluorescence quencher (MGB-NFQ) or ZEN-IB and Iowa Black Fluorescence quencher (IBFQ) at the 3′ end. In one embodiment the primer probe combination detects DNA encoding the structural capsid (VP) protein and/or the non-structural (NS) protein of Bovine parvovirus 3 in a test sample. In one embodiment the reagent in combination with an internal positive control primer combination. In one embodiment the reagent comprises SEQ ID NO:7, SEQ ID NO:8, and SEQ ID NO:9.

The invention provides a reagent for use as an internal positive control primer probe combination in an assay for detecting Bovine parvovirus 3 (BPV-3) genomic DNA in the extracted DNA of a test sample comprising a primer probe combination. In a related embodiment the internal positive control primer probe combination has the nucleic acid sequences of SEQ ID NO: 16, SEQ ID NO:17, and SEQ ID NO:18. In one embodiment SEQ ID NO: 18 has a fluorescent reporter dye and/or a non-fluorescent quencher. In a related embodiment SEQ ID NO: 18 has a fluorescent reporter dye, 2′-chloro-7′phenyl-1,4-dichloro-6-carboxy-fluorescein (VIC) at the 5′ end and a Minor Groove Binder non-fluorescent quencher (MGB-NFQ) at the 3′ end. In one embodiment the reagent comprises SEQ ID NO:7, SEQ ID NO:8, and SEQ ID NO:9.

The invention provides a primer probe combination for detecting Bovine parvovirus 3 genomic DNA in the extracted DNA of a test sample in combination with an internal positive control primer probe combination for detecting Bovine parvovirus 3 (BPV-3) genomic DNA. In one embodiment the primer probe combination is selected from the groups consisting of a) a primer probe combination having the nucleic acid sequences of SEQ ID NO:1, SEQ ID NO:2, and SEQ ID NO:3; b) a primer probe combination having the nucleic acid sequences of SEQ ID NO:4, SEQ ID NO:5, and SEQ ID NO:6; c) a primer probe combination having the nucleic acid sequences of SEQ ID NO:7, SEQ ID NO:8, and SEQ ID NO:9; d) a primer probe combination having the nucleic acid sequences of SEQ ID NO:10, SEQ ID NO:11, and SEQ ID NO:12; e) a primer probe combination having the nucleic acid sequences of SEQ ID NO:13, SEQ ID NO:14, and SEQ ID NO:15, f) a primer probe combination having the nucleic acid sequences of SEQ ID NO:20, SEQ ID NO:21, and SEQ ID NO:22; g) a primer probe combination having the nucleic acid sequences of SEQ ID NO:23, SEQ ID NO:24, and SEQ ID NO:25; and h) a primer probe combination having the nucleic acid sequences of SEQ ID NO:26, SEQ ID NO:27, and SEQ ID NO:28. In one embodiment SEQ ID NO: 3, SEQ ID NO:6, SEQ ID NO:9, SEQ ID NO:12, or SEQ ID NO:15 have a fluorescent reporter dye and/or a non-fluorescent quencher. In one embodiment one or more of SEQ ID NO: 3, SEQ ID NO:6, SEQ ID NO:9, SEQ ID NO:12, SEQ ID NO:15, SEQ ID NO:22, SEQ ID NO:25; or SEQ ID NO:28 have a fluorescent reporter dye 6-carboxyfluorescein (FAM) at the 5′ end and/or either a Minor Groove Binder non-fluorescence quencher (MGB-NFQ) or ZEN-IB and Iowa Black Fluorescence quencher (IBFQ) at the 3′ end. In one embodiment the primer probe combination detects DNA encoding the structural capsid (VP) protein and/or the non-structural (NS) protein of Bovine parvovirus 3 in a test sample. In one embodiment an internal positive control primer probe combination having the nucleic acid sequences of SEQ ID NO: 16, SEQ ID NO:17, and SEQ ID NO:18 is used in combination with a primer probe combination used for detecting Bovine parvovirus 3 (BPV-3) genomic DNA in a test sample as described above. In a related embodiment SEQ ID NO: 20 has a fluorescent reporter dye and/or a non-fluorescent quencher. In a related embodiment SEQ ID NO: 18 has a fluorescent reporter dye, 2′-chloro-7′phenyl-1,4-dichloro-6-carboxy-fluorescein (VIC) at the 5′ end and a Minor Groove Binder non-fluorescent quencher (MGB-NFQ) at the 3′ end.

The invention provides a primer probe combination for detecting DNA encoding Bovine parvovirus 3 genomic DNA in the extracted DNA of a test sample comprising SEQ ID NO:7, SEQ ID NO:8, and SEQ ID NO:9 in combination with an internal positive control primer probe combination comprising SEQ ID NO: 16, SEQ ID NO:17, and SEQ ID NO:18, wherein SEQ ID NO: 9 has a fluorescent reporter dye 6-carboxyfluorescein at the 5′ end and a Minor Groove Binder non-fluorescent quencher (MGB-NFQ) non-fluorescence quencher at the 3′ end and SEQ ID NO:18 has a fluorescent reporter dye, 2′-chloro-7′phenyl-1,4-dichloro-6-carboxy-fluorescein at the 5′ end and a Minor Groove Binder non-fluorescent quencher (MGB-NFQ) at the 3′ end.

The invention provides a kit for detecting Bovine parvovirus 3 (BPV-3) genomic DNA contamination in the extracted DNA of a test sample comprising primer probe combination that detects DNA encoding BPV-3 selected from the group consisting of a) a primer probe combination having the nucleic acid sequences of SEQ ID NO:1, SEQ ID NO:2, and SEQ ID NO:3; b) a primer probe combination having the nucleic acid sequences of SEQ ID NO:4, SEQ ID NO:5, and SEQ ID NO:6; c) a primer probe combination having the nucleic acid sequences of SEQ ID NO:7, SEQ ID NO:8, and SEQ ID NO:9; d) a primer probe combination having the nucleic acid sequences of SEQ ID NO:10, SEQ ID NO:11, and SEQ ID NO:12; and e) a primer probe combination having the nucleic acid sequences of SEQ ID NO:13, SEQ ID NO:14, SEQ ID NO:15, f) a primer probe combination having the nucleic acid sequences of SEQ ID NO:20, SEQ ID NO:21, and SEQ ID NO:22; g) a primer probe combination having the nucleic acid sequences of SEQ ID NO:23, SEQ ID NO:24, and SEQ ID NO:25; and h) a primer probe combination having the nucleic acid sequences of SEQ ID NO:26, SEQ ID NO:27, and SEQ ID NO:28; and optionally an internal positive control primer probe combination having the nucleic acid sequences of SEQ ID NO: 16, SEQ ID NO:17, and SEQ ID NO:18. In one embodiment the kit comprises a primer probe combination that detects DNA encoding BPV-3 selected from the group consisting of a) a primer probe combination having the nucleic acid sequences of SEQ ID NO:1, SEQ ID NO:2, and SEQ ID NO:3; b) a primer probe combination having the nucleic acid sequences of SEQ ID NO:4, SEQ ID NO:5, and SEQ ID NO:6; c) a primer probe combination having the nucleic acid sequences of SEQ ID NO:7, SEQ ID NO:8, and SEQ ID NO:9; d) a primer probe combination having the nucleic acid sequences of SEQ ID NO:10, SEQ ID NO:11, and SEQ ID NO:12; and e) a primer probe combination having the nucleic acid sequences of SEQ ID NO:13, SEQ ID NO:14, and SEQ ID NO:15, f) a primer probe combination having the nucleic acid sequences of SEQ ID NO:20, SEQ ID NO:21, and SEQ ID NO:22; g) a primer probe combination having the nucleic acid sequences of SEQ ID NO:23, SEQ ID NO:24, and SEQ ID NO:25; and h) a primer probe combination having the nucleic acid sequences of SEQ ID NO:26, SEQ ID NO:27, and SEQ ID NO:28; and an internal positive control primer probe combination having the nucleic acid sequences of SEQ ID NO: 16, SEQ ID NO:17, and SEQ ID NO:18.

The invention provides a method for determining the presence or absence of Bovine parvovirus 3 genomic DNA in the extracted DNA of a test sample comprising 1) a reaction mixture comprising a test sample, a positive control, a BPV-3_IPC positive control plasmid DNA, nucleic acid amplification reagents, an internal positive control primer probe combination having the nucleic acid sequences of SEQ ID NO: 16, SEQ ID NO:17, and SEQ ID NO:18, wherein SEQ ID NO:18 has a fluorescent reporter dye at the 5′ end and a non-fluorescence quencher at the 3′ end, and a primer probe combination selective for a DNA sequence of Bovine parvovirus 3, selected from the group consisting of a) a primer probe combination having the nucleic acid sequences of SEQ ID NO:1, SEQ ID NO:2, and SEQ ID NO:3, wherein SEQ ID NO:3 has a fluorescent reporter dye at the 5′ end and a non-fluorescence quencher at the 3′ end; b) a primer probe combination having the nucleic acid sequences of SEQ ID NO:4, SEQ ID NO:5, and SEQ ID NO:6, wherein SEQ ID NO:6 has a fluorescent reporter dye at the 5′ end and a non-fluorescence quencher at the 3′ end; c) a primer probe combination having the nucleic acid sequences of SEQ ID NO:7, SEQ ID NO:8, and SEQ ID NO:9, wherein SEQ ID NO:9 has a fluorescent reporter dye at the 5′ end and a non-fluorescence quencher at the 3′ end; d) a primer probe combination having the nucleic acid sequences of SEQ ID NO:10, SEQ ID NO:11, and SEQ ID NO:12, wherein SEQ ID NO:12 has a fluorescent reporter dye at the 5′ end and a non-fluorescence quencher at the 3′ end; e) a primer probe combination having the nucleic acid sequences of SEQ ID NO:13, SEQ ID NO:14, and SEQ ID NO:15, wherein SEQ ID NO:15 has a fluorescent reporter dye at the 5′ end and a non-fluorescence quencher at the 3′ end; g) a primer probe combination having the nucleic acid sequences of SEQ ID NO:23, SEQ ID NO:24, and SEQ ID NO:25, wherein SEQ ID NO:25 has a fluorescent reporter dye at the 5′ end and a non-fluorescence quencher at the 3′ end; and h) a primer probe combination having the nucleic acid sequences of SEQ ID NO:26, SEQ ID NO:27, and SEQ ID NO:28, wherein SEQ ID NO:28 has a fluorescent reporter dye at the 5′ end and a non-fluorescence quencher at the 3′ end; 2) subjecting the reaction mixture to a quantitative PCR technique to obtain copies of the target sequence, 2) subjecting the reaction mixture to a quantitative PCR technique to obtain copies of the target sequence, 3) measuring any increase in fluorescence signal, wherein an increase in fluorescence signal indicates the presence of Bovine parvovirus 3 genomic DNA in the test sample. In one embodiment the fluorescent reporter dye is 6-carboxyfluorescein (FAM) and the non-fluorescence quencher is a Minor Groove Binder non-fluorescent quencher (MGB-NFQ) or ZEN-IB and Iowa Black Fluorescence quencher (IBFQ). In one embodiment the invention further comprises one or more negative extraction control, no template control, positive extraction control, positive control, and/or inhibition control. In one embodiment the sensitivity or analytical limit of detection is 22 genome copies per reaction. In one embodiment the sample limit of detection is 25 genome copies per reaction. In one embodiment the primer probe combination selective for a DNA sequence of Bovine parvovirus 3 detects genomic DNA encoding the non-structural (NS) protein and/or structural capsid (VP) protein of Bovine parvovirus 3. In one embodiment the primer probe combination selective for a DNA sequence of Bovine parvovirus 3 amplifies a 144 bp fragment. In one embodiment the primer probe combination selective for a DNA sequence of Bovine parvovirus 3 comprises the combination of SEQ ID NO:7, SEQ ID NO:8, and SEQ ID NO:9, wherein SEQ ID NO:9 has a 6-carboxyfluorescein (FAM) at the 5′ end and a Minor Groove Binder non-fluorescent quencher (MGB-NFQ) at the 3′ end. In one embodiment the method further comprises an internal positive control primer probe combination. In a related embodiment the primer probe combination has the nucleic acid sequences of SEQ ID NO: 16, SEQ ID NO:17, and SEQ ID NO:18. In a related embodiment SEQ ID NO: 18 has a fluorescent reporter dye and/or a non-fluorescent quencher. In a related embodiment SEQ ID NO: 18 has a fluorescent reporter dye, 2′-chloro-7′phenyl-1,4-dichloro-6-carboxy-fluorescein (VIC) at the 5′ end and a Minor Groove Binder non-fluorescent quencher (MGB-NFQ) at the 3′ end.

The invention provides a method for the quantification of 1E3 to 1E8 genome copies of Bovine parvovirus 3 genomic DNA in a PCR reaction comprising 1) a reaction mixture comprising the extracted DNA of a test sample, a positive control, a BPV-3 IPC positive control plasmid DNA, nucleic acid amplification reagents, an internal positive control primer probe combination having the nucleic acid sequences of SEQ ID NO: 16, SEQ ID NO:17, and SEQ ID NO:18, wherein SEQ ID NO:18 has a fluorescent reporter dye at the 5′ end and a non-fluorescence quencher at the 3′ end, and a primer probe combination selective for a DNA sequence of Bovine parvovirus 3, selected from the group consisting of a) a primer probe combination having the nucleic acid sequences of SEQ ID NO:1, SEQ ID NO:2, and SEQ ID NO:3, wherein SEQ ID NO:3 has a fluorescent reporter dye at the 5′ end and a non-fluorescence quencher at the 3′ end; b) a primer probe combination having the nucleic acid sequences of SEQ ID NO:4, SEQ ID NO:5, and SEQ ID NO:6, wherein SEQ ID NO:6 has a fluorescent reporter dye at the 5′ end and a non-fluorescence quencher at the 3′ end; c) a primer probe combination having the nucleic acid sequences of SEQ ID NO:7, SEQ ID NO:8, and SEQ ID NO:9, wherein SEQ ID NO:9 has a fluorescent reporter dye at the 5′ end and a non-fluorescence quencher at the 3′ end; d) a primer probe combination having the nucleic acid sequences of SEQ ID NO:10, SEQ ID NO:11, and SEQ ID NO:12, wherein SEQ ID NO:12 has a fluorescent reporter dye at the 5′ end and a non-fluorescence quencher at the 3′ end; e) a primer probe combination having the nucleic acid sequences of SEQ ID NO:13, SEQ ID NO:14, and SEQ ID NO:15, wherein SEQ ID NO:15 has a fluorescent reporter dye at the 5′ end and a non-fluorescence quencher at the 3′ end; f) a primer probe combination having the nucleic acid sequences of SEQ ID NO:20, SEQ ID NO:21, and SEQ ID NO:22, wherein SEQ ID NO:22 has a fluorescent reporter dye at the 5′ end and a non-fluorescence quencher at the 3′ end; g) a primer probe combination having the nucleic acid sequences of SEQ ID NO:23, SEQ ID NO:24, and SEQ ID NO:25, wherein SEQ ID NO:25 has a fluorescent reporter dye at the 5′ end and a non-fluorescence quencher at the 3′ end; and h) a primer probe combination having the nucleic acid sequences of SEQ ID NO:26, SEQ ID NO:27, and SEQ ID NO:28, wherein SEQ ID NO:28 has a fluorescent reporter dye at the 5′ end and a non-fluorescence quencher at the 3′ end; 2) subjecting the reaction mixture to a quantitative PCR technique to obtain copies of the target sequence, and 3) measuring any increase in fluorescence signal. In one embodiment the limit of detection (LOD95%) of the method is 27 genome copies of Bovine parvovirus 3 genomic DNA per reaction with a 95% confidence interval of 22 and 34 genome copies per reaction. In one embodiment the linearity of the method has a correlation coefficient (R2)≥0.98 and a PCR amplification efficiency within 90-110%. In one embodiment the method has a repeatability value that is a % CV of quantity equal or less than 25%. In one embodiment the method has an intermediate precision value that is % CV of quantity equal or less than 30%. In one embodiment the method has an accuracy value within ±30% of the accepted reference value (ST) across the whole dynamic range of the assay. In one embodiment the method has a limit of quantitation that is the % CV of quantity for repeatability at ≤25%, intermediate precision at ≤30% and acceptance criterion for the accuracy within ±30% of the expected standard reference value. In another embodiment the method has a robustness that has a percent CV of quantity for repeatability of ≤25%, an intermediate precision of ≤30%, and an accuracy of the mean of quantity of the combination matrix condition tested of ±30% of the mean of quantity of the optimized condition. In another embodiment the method includes one or more of a no template control, a positive control, a negative extraction control, a positive extraction control, an inhibition control, an internal positive control, and a standard.

### BRIEF DESCRIPTION OF THE DRAWINGS

FIG. 1. The Probit analysis used for determination of ALOD in BPV-3 qPCR with 95% of probability (LOD95%).

FIG. 2. POD graph and LOD95% analysis results.

### DETAILED DESCRIPTION OF THE INVENTION

As described herein, a qualitative real-time PRC method has been developed to detect potential Bovine parvovirus 3 (BPV-3) genomic DNA contamination in the extracted of test samples. Qualification parameters, including specificity, limit of detection (LOD), robustness and repeatability were used to test and confirm the performance of the assay.

The assay makes use of optimized primer probe combinations for detecting Bovine parvovirus 3 (BPV-3) genomic DNA in the extracted DNA of a test sample. The BPV-3 PCR primers amplify conserved regions in the genes encoding the BPV-3 non-structural (NS) protein and/or structural capsid (VP) protein in the BPV-3 genome. “Method Validation of U.S. Environmental Protection Agency (EPA) Microbiological Methods of Analysis”, REVISION: Dec. 21, 2016, was consulted and considered in the design and development of the assay.

The invention provides oligonucleotide primer probe combinations that can be used in detection methods, such as in quantitative polymerase chain reaction (qPCR) techniques, to detect DNA encoding Bovine parvovirus 3 genomic (BPV-3) and for use in methods for determining the presence or absence of BPV-3 in the extracted DNA of a test sample.

As used herein, “oligonucleotides” are short single stranded synthetic DNA or RNA molecules, less than 200 nucleotides in length, typically in the range of 13-25 nucleotides in length. Oligonucleotides bind to their complement oligonucleotide to form a duplex. As a result, oligonucleotides are commonly used in a variety of applications where detection of the presence or absence of specific DNA or RNA sequences is desired. In particular, oligonucleotides are useful as primers for use in polymerase chain reactions (PCR). Methods to synthesize oligonucleotides are known in the art, equipment to produce oligonucleotides are commercially available and there are service providers who will make custom oligonucleotides on demand. Oligonucleotides can also be obtained from the breakdown of larger nucleic acid molecules, or naturally occurring oligonucleotides, such as micro RNA.

The invention provides a composition comprising oligonucleotides selected from the groups consisting of a) oligonucleotides having the nucleic acid sequence of SEQ ID NO:1, SEQ ID NO:2, and SEQ ID NO:3; b) oligonucleotides having the nucleic acid sequence of SEQ ID NO:4, SEQ ID NO:5, and SEQ ID NO:6; c) oligonucleotides having the nucleic acid sequence of SEQ ID NO:7, SEQ ID NO:8, and SEQ ID NO:9; d) oligonucleotides having the nucleic acid sequence of SEQ ID NO:10, SEQ ID NO:11, and SEQ ID NO:12; e) oligonucleotides having the nucleic acid sequence of SEQ ID NO:13, SEQ ID NO:14, and SEQ ID NO:15, f) oligonucleotides having the nucleic acid sequence of SEQ ID NO:20, SEQ ID NO:21, and SEQ ID NO:22, g) oligonucleotides having the nucleic acid sequence of SEQ ID NO:23, SEQ ID NO:24, and SEQ ID NO:25, and h) oligonucleotides having the nucleic acid sequence of SEQ ID NO:26, SEQ ID NO:27, and SEQ ID NO:28. In one embodiment the composition comprises oligonucleotides having the nucleic acid sequence of SEQ ID NO:7, SEQ ID NO:8, and SEQ ID NO:9.

In one embodiment the composition optionally comprises a second composition comprising oligonucleotides having the sequences of SEQ ID NO: 16, SEQ ID NO:17, and SEQ ID NO:18 in combination with one or more of the primer probe combination mentioned above. In one embodiment the composition comprises oligonucleotides having the nucleic acid sequence of SEQ ID NO:7, SEQ ID NO:8, SEQ ID NO:9, SEQ ID NO: 16, SEQ ID NO:17, and SEQ ID NO:18. In one embodiment the composition comprises a first composition comprises oligonucleotides having the nucleic acid sequence of SEQ ID NO:7, SEQ ID NO:8, and SEQ ID NO:9 and a second composition comprises oligonucleotides having the sequence of SEQ ID NO: 16, SEQ ID NO:17, and SEQ ID NO:18.

In one embodiment one or more of SEQ ID NO: 3, SEQ ID NO:6, SEQ ID NO:9, SEQ ID NO:12, SEQ ID NO:15, SEQ ID NO:18, SEQ ID NO: 22, SEQ ID NO: 25, and SEQ ID NO: 28, may include at least one fluorescent reporter dye at the 5′ end and/or at least one non-fluorescence quencher at the 3′ end. In one embodiment one or more of SEQ ID NO: 3, SEQ ID NO:6, SEQ ID NO:9, SEQ ID NO:12, SEQ ID NO:15, SEQ ID NO:18, SEQ ID NO: 22, SEQ ID NO: 25, and SEQ ID NO: 28, have at least one fluorescent reporter dye at the 5′ end and at least one non-fluorescence quencher at the 3′ end. In one embodiment one or more of SEQ ID NO: 3, SEQ ID NO:6, SEQ ID NO:9, SEQ ID NO:12, SEQ ID NO:15, SEQ ID NO:18, SEQ ID NO: 22, SEQ ID NO: 25, and SEQ ID NO: 28, have a fluorescent reporter dye at the 5′ end and a non-fluorescence quencher at the 3′ end.

In one embodiment one or more of SEQ ID NO: 3, SEQ ID NO:6, SEQ ID NO:9, SEQ ID NO:12, SEQ ID NO:15, SEQ ID NO:18, SEQ ID NO: 22, SEQ ID NO: 25, and SEQ ID NO: 28, have a fluorescent reporter dye selected from 6-carboxyfluorescein (FAM) or 2′-chloro-7′phenyl-1,4-dichloro-6-carboxy-fluorescein (VIC) at the 5′ end. In one embodiment SEQ ID NO: 3, SEQ ID NO:6, SEQ ID NO:9, SEQ ID NO:12, SEQ ID NO:15, SEQ ID NO: 22, SEQ ID NO: 25, and SEQ ID NO: 28, have a fluorescent reporter dye 6-carboxyfluorescein (FAM) at the 5′ end. In one embodiment, SEQ ID NO:18 has the fluorescent reporter dye 2′-chloro-7′phenyl-1,4-dichloro-6-carboxy-fluorescein (VIC) at the 5′ end.

In one embodiment one or more of SEQ ID NO:3, SEQ ID NO:6, SEQ ID NO:9, SEQ ID NO:12, SEQ ID NO:15, SEQ ID NO: 18, SEQ ID NO: 22, SEQ ID NO: 25, and SEQ ID NO: 28, have a non-fluorescent quencher at the 3′ end. In one embodiment the quencher is selected from a Minor Groove Binder non-fluorescent quencher (MGB-NFQ) and ZEN-IB and Iowa Black Fluorescence quencher (IBFQ) at the 3′ end. In one embodiment one or more of SEQ ID NO:6, SEQ ID NO:9, SEQ ID NO:12, SEQ ID NO:15, and SEQ ID NO: 18 have a Minor Groove Binder non-fluorescent quencher (MGB-NFQ) at the 3′ end. In one embodiment one or more of SEQ ID NO: 3, SEQ ID NO: 22, SEQ ID NO: 25, and SEQ ID NO: 28, has ZEN-IB and Iowa Black Fluorescence quencher (IBFQ) at the 3′end.

In one embodiment SEQ ID NO: 3 has a fluorescent reporter dye 6-carboxyfluorescein (FAM) at the end and ZEN-IB and Iowa Black Fluorescence quencher (IBFQ) at the 3′ end. In one embodiment SEQ ID NO: 6 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 9 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 12 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 15 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 18 has a fluorescent reporter dye 2′-chloro-7′phenyl-1,4-dichloro-6-carboxy-fluorescein (VIC) at the 5′ end and a Minor Groove Binder non-fluorescent quencher (MGB-NFQ) at the 3′ end. In one embodiment SEQ ID NO: 22 has a fluorescent reporter dye 6-carboxyfluorescein (FAM) at the 5′ end and ZEN-IB and Iowa Black Fluorescence quencher (IBFQ) at the 3′ end. In one embodiment SEQ ID NO: 25 has a fluorescent reporter dye 6-carboxyfluorescein (FAM) at the 5′ end and ZEN-IB and Iowa Black Fluorescence quencher (IBFQ) at the 3′ end. In one embodiment SEQ ID NO: 28 has a fluorescent reporter dye 6-carboxyfluorescein (FAM) at the 5′ end and ZEN-IB and Iowa Black Fluorescence quencher (IBFQ) at the 3′ end.

The invention provides a reagent for use in detecting Bovine parvovirus 3 (BPV-3) genomic DNA. This reagent can be used for determining the presence or absence of Bovine parvovirus 3 genomic DNA in the extracted DNA of a test sample.

As used herein, “test sample” means any sample of extracted DNA for which a determination of the presence or absence of BPV-3 is desired. The test sample may be known or suspected to contain BPV-3. Test samples may come from raw materials, such as those used in the manufacture of biotherapeutics. Raw materials include those known or suspected of having an animal origin or contain components from an animal origin, particularly a bovine origin or known or suspected of having been in contact with other materials that have an animal, particularly a bovine origin, including such raw materials as fetal bovine serum and fetal calf serum. Test samples may also come from cell culture media, particularly cell culture media containing fetal bovine or fetal calf serum. The media can be obtained prior to or during cell culture, or after harvest of cell culture media from a cell culture operation. Periodic samples may be taken during cell culture, this may be multiple times a day, daily, at critical points during the culture, particularly at the start and harvest of the culture. Test samples may also come from cell lines used in the manufacture of biotherapeutics, including cells and cell lines of a bovine origin. Test samples may also come from samples taken during downstream processing, for example from the eluate from downstream purification steps, samples can be taken from the drug substance, and from the drug product.

The invention provides a reagent for detecting Bovine parvovirus 3 (BPV-3) genomic DNA in the extracted DNA of a test sample selected from the group consisting of a) a primer probe combination having the nucleic acid sequences of SEQ ID NO:1, SEQ ID NO:2, and SEQ ID NO:3; b) a primer probe combination having the nucleic acid sequences of SEQ ID NO:4, SEQ ID NO:5, and SEQ ID NO:6; c) a primer probe combination having the nucleic acid sequences of SEQ ID NO:7, SEQ ID NO:8, and SEQ ID NO:9; d) a primer probe combination having the nucleic acid sequences of SEQ ID NO:10, SEQ ID NO:11, and SEQ ID NO:12; and e) a primer probe combination having the nucleic acid sequences of SEQ ID NO:13, SEQ ID NO:14, and SEQ ID NO:15. In one embodiment the primer probe combination comprises the nucleic acid sequence of SEQ ID NO:7, SEQ ID NO:8, and SEQ ID NO:9.

In one embodiment, the reagent optionally includes an internal positive control primer probe combination. In one embodiment, the reagent further comprises an internal positive control primer probe combination. In one embodiment the internal positive control primer probe combination comprises the nucleic acid of SEQ ID NO: 16, SEQ ID NO:17, and SEQ ID NO:18. In one embodiment the reagent a primer probe combination having the nucleic acid sequence of SEQ ID NO:7, SEQ ID NO:8, SEQ ID NO:9, and a primer probe combination having the nucleic acid sequence of SEQ ID NO: 16, SEQ ID NO:17, and SEQ ID NO:18.

In one embodiment one or more of SEQ ID NO: 3, SEQ ID NO:6, SEQ ID NO:9, SEQ ID NO:12, SEQ ID NO:15, or SEQ ID NO:18 may include at least one fluorescent reporter dye at the 5′ end and/or at least one non-fluorescence quencher at the 3′ end. In a related embodiment one or more of SEQ ID NO: 3, SEQ ID NO:6, SEQ ID NO:9, SEQ ID NO:12, SEQ ID NO:15 or SEQ ID NO:18 have at least one fluorescent reporter dye at the 5′ end and at least one non-fluorescence quencher at the 3′ end. In a related embodiment one or more of SEQ ID NO: 3, SEQ ID NO:6, SEQ ID NO:9, SEQ ID NO:12, SEQ ID NO:15, and SEQ ID NO:18 have a fluorescent reporter dye at the 5′ end and a non-fluorescence quencher at the 3′ end.

In one embodiment one or more of SEQ ID NO: 3, SEQ ID NO:6, SEQ ID NO:9, SEQ ID NO:12, SEQ ID NO:15, and SEQ ID NO:18 have a fluorescent reporter dye selected from 6-carboxyfluorescein (FAM) or 2′-chloro-7′phenyl-1,4-dichloro-6-carboxy-fluorescein (VIC) at the 5′ end. In one embodiment SEQ ID NO: 3, SEQ ID NO:6, SEQ ID NO:9, SEQ ID NO:12, and SEQ ID NO:15 have a fluorescent reporter dye 6-carboxyfluorescein (FAM) at the 5′ end. In one embodiment, SEQ ID NO:18 has the fluorescent reporter dye 2′-chloro-7′phenyl-1,4-dichloro-6-carboxy-fluorescein (VIC) at the 5′ end.

In one embodiment one or more of SEQ ID NO:3, SEQ ID NO:6, SEQ ID NO:9, SEQ ID NO:12, SEQ ID NO:15, or SEQ ID NO: 18 have a non-fluorescent quencher at the 3′ end. In one embodiment the quencher is selected from a Minor Groove Binder non-fluorescent quencher (MGB-NFQ) and ZEN-IB and Iowa Black Fluorescence quencher (IBFQ) at the 3′ end. In one embodiment one or more of SEQ ID NO:6, SEQ ID NO:9, SEQ ID NO:12, SEQ ID NO:15, and SEQ ID NO: 18 have a Minor Groove Binder non-fluorescent quencher (MGB-NFQ) at the 3′ end. In one embodiment SEQ ID NO: 3 has ZEN-IB and Iowa Black Fluorescence quencher (IBFQ) at the 3′end.

In one embodiment SEQ ID NO: 3 has a fluorescent reporter dye 6-carboxyfluorescein (FAM) at the end and ZEN-IB and Iowa Black Fluorescence quencher (IBFQ) at the 3′ end. In one embodiment SEQ ID NO: 6 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 9 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 12 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 15 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 18 has a fluorescent reporter dye 2′-chloro-7′phenyl-1,4-dichloro-6-carboxy-fluorescein (VIC) at the 5′ end and a Minor Groove Binder non-fluorescent quencher (MGB-NFQ) at the 3′ end.

The invention provides primer probe combinations that targets the structural capsid (VP) protein of BPV-3. In one embodiment, the primer probe combination comprises a forward primer that targets oligo position 3535-3554 of the BPV-3 isolate having the GenBank accession number AF406967, a reverse primer that targets oligo position 3667-3643 of the BPV-3 isolate having the GenBank accession number AF406967, and a probe that targets oligo position 3571-3588 of the BPV-3 isolate having the GenBank accession number AF406967. In one embodiment, the primer probe combination the forward primer has the nucleic acid sequence of SEQ ID NO:1, the reverse primer has the nucleic acid sequence of SEQ ID NO:2, and the probe has the nucleic acid sequence of SEQ ID NO:3. In one embodiment, the primer probe combination comprises a forward primer that targets oligo position 1734-1716 of the BPV-3 isolate having the GenBank accession number AF406967, a reverse primer that targets oligo position 1618-1645 of the BPV-3 isolate having the GenBank accession number AF406967, and a probe that targets oligo position 1665-1678 of the BPV-3 isolate having the GenBank accession number AF406967. In one embodiment, the primer probe combination the forward primer has the nucleic acid sequence of SEQ ID NO:13, the reverse primer has the nucleic acid sequence of SEQ ID NO:14, and the probe has the nucleic acid sequence of SEQ ID NO:15. In one embodiment, the primer probe combination comprises a forward primer that targets oligo position 2862-2878 of the BPV-3 isolate having the GenBank accession number AF406967, a reverse primer that targets oligo position 2963-2938 of the BPV-3 isolate having the GenBank accession number AF406967, and a probe that targets oligo position 2902-2930 of the BPV-3 isolate having the GenBank accession number AF406967. In one embodiment, the primer probe combination the forward primer has the nucleic acid sequence of SEQ ID NO:20, the reverse primer has the nucleic acid sequence of SEQ ID NO:21, and the probe has the nucleic acid sequence of SEQ ID NO:22. In one embodiment, the primer probe combination comprises a forward primer that targets oligo position 3051-3068 of the BPV-3 isolate having the GenBank accession number AF406967, a reverse primer that targets oligo position 3134-3114 of the BPV-3 isolate having the GenBank accession number AF406967, and a probe that targets oligo position 3079-3102 of the BPV-3 isolate having the GenBank accession number AF406967. In one embodiment, the primer probe combination the forward primer has the nucleic acid sequence of SEQ ID NO:23, the reverse primer has the nucleic acid sequence of SEQ ID NO:24, and the probe has the nucleic acid sequence of SEQ ID NO:25. In one embodiment, the primer probe combination comprises a forward primer that targets oligo position 3061-3079 of the BPV-3 isolate having the GenBank accession number AF406967, a reverse primer that targets oligo position 3140-3122 of the BPV-3 isolate having the GenBank accession number AF406967, and a probe that targets oligo position 3081-3103 of the BPV-3 isolate having the GenBank accession number AF406967. In one embodiment, the primer probe combination the forward primer has the nucleic acid sequence of SEQ ID NO:26, the reverse primer has the nucleic acid sequence of SEQ ID NO:27, and the probe has the nucleic acid sequence of SEQ ID NO:28.

The invention provides primer probe combinations that targets the non-structural (NS) protein and structural capsid (VP) protein of BPV-3. In one embodiment, the primer probe combination comprises a forward primer that targets oligo position 2190-2209 of the BPV-3 isolate having the GenBank accession number AF406967, a reverse primer that targets oligo position 2256-2236 of the BPV-3 isolate having the GenBank accession number AF406967, and a probe that targets oligo position 2213-2226 of the BPV-3 isolate having the GenBank accession number AF406967. In one embodiment the forward primer has the nucleic acid sequence of SEQ ID NO:4, the reverse primer has the nucleic acid sequence of SEQ ID NO:5, and the probe has the nucleic acid sequence of SEQ ID NO:6.

The invention provides primer probe combinations that targets the non-structural (NS) protein of BPV-3. In one embodiment, the primer probe combination comprises a forward primer that targets oligo position 1331-1352 of the BPV-3 isolate having the GenBank accession number AF406967, a reverse primer that targets oligo position 1474-1453 of the BPV-3 isolate having the GenBank accession number AF406967, and a probe that targets oligo position 1377-1391 of the BPV-3 isolate having the GenBank accession number AF406967. In one embodiment the forward primer has the nucleic acid sequence of SEQ ID NO:7, the reverse primer has the nucleic acid sequence of SEQ ID NO:8, and the probe has the nucleic acid sequence of SEQ ID NO:9. In one embodiment, the primer probe combination comprises a forward primer that targets oligo position 1453-1474 of the BPV-3 isolate having the GenBank accession number AF406967, a reverse primer that targets oligo position 1562-1539 of the BPV-3 isolate having the GenBank accession number AF406967, and a probe that targets oligo position 1507-1522 of the BPV-3 isolate having the GenBank accession number AF406967. In one embodiment the forward primer has the nucleic acid sequence of SEQ ID NO:10, the reverse primer has the nucleic acid sequence of SEQ ID NO:11, and the probe has the nucleic acid sequence of SEQ ID NO:12.

In one embodiment, the reagent for detecting BPV-3 genomic DNA in a sample is used in combination with an internal positive control primer combination. The invention provides a primer probe combination for detecting DNA encoding Bovine parvovirus 3 genomic DNA in the extracted DNA of a test sample in combination with an internal positive control primer probe combination comprising SEQ ID NO: 16, SEQ ID NO:17, and SEQ ID NO:18. The invention provides a primer probe combination for detecting DNA encoding Bovine parvovirus 3 genomic DNA in the extracted DNA of a test sample comprising SEQ ID NO:7, SEQ ID NO:8, and SEQ ID NO:9 in combination with an internal positive control primer probe combination comprising SEQ ID NO: 16, SEQ ID NO:17, and SEQ ID NO:18, wherein SEQ ID NO: 9 has a fluorescent reporter dye 6-carboxyfluorescein at the 5′ end and a Minor Groove Binder non-fluorescent quencher (MGB-NFQ) non-fluorescence quencher at the 3′ end and SEQ ID NO:18 has a fluorescent reporter dye, 2′-chloro-7′phenyl-1,4-dichloro-6-carboxy-fluorescein at the 5′ end and a Minor Groove Binder non-fluorescent quencher (MGB-NFQ) at the 3′ end. In one embodiment SEQ ID NO:18 has a fluorescent reporter dye and/or a non-fluorescent quencher. In one embodiment SEQ ID NO:18 has a fluorescent reporter dye 2′-chloro-7′phenyl-1,4-dichloro-6-carboxy-fluorescein (VIC) at the 5′ end and a Minor Groove Binder non-fluorescent quencher (MGB-NFQ) at the 3′ end.

The invention provides a primer probe combination for detecting Bovine parvovirus 3 genomic DNA in the extracted DNA of a test sample in combination with an internal positive control primer probe combination for detecting Bovine parvovirus 3 (BPV-3) genomic DNA. In one embodiment the primer probe combination for detecting Bovine parvovirus 3 genomic DNA as described above wherein the primer probe combination is selected from the groups consisting of a) a primer probe combination having the nucleic acid sequences of SEQ ID NO:1, SEQ ID NO:2, and SEQ ID NO:3; b) a primer probe combination having the nucleic acid sequences of SEQ ID NO:4, SEQ ID NO:5, and SEQ ID NO:6; c) a primer probe combination having the nucleic acid sequences of SEQ ID NO:7, SEQ ID NO:8, and SEQ ID NO:9; d) a primer probe combination having the nucleic acid sequences of SEQ ID NO:10, SEQ ID NO:11, and SEQ ID NO:12; e) a primer probe combination having the nucleic acid sequences of SEQ ID NO:13, SEQ ID NO:14, and SEQ ID NO:15; f) a primer probe combination having the nucleic acid sequences of SEQ ID NO:20, SEQ ID NO:21, and SEQ ID NO:22; g) a primer probe combination having the nucleic acid sequences of SEQ ID NO:23, SEQ ID NO:24, and SEQ ID NO:25; and h) a primer probe combination having the nucleic acid sequences of SEQ ID NO:26, SEQ ID NO:27, and SEQ ID NO:28. In one embodiment the primer probe combination wherein SEQ ID NO: 3, SEQ ID NO:6, SEQ ID NO:9, SEQ ID NO:12, SEQ ID NO:15, SEQ ID NO:22, SEQ ID NO:25, and SEQ ID NO:28 have a fluorescent reporter dye and/or a non-fluorescent quencher. In one embodiment, the primer probe combination wherein one or more of SEQ ID NO: 3, SEQ ID NO:6, SEQ ID NO:9, SEQ ID NO:12, SEQ ID NO:15, SEQ ID NO:22, SEQ ID NO:25, and SEQ ID NO:28 have a fluorescent reporter dye 6-carboxyfluorescein (FAM) at the 5′ end and/or either a Minor Groove Binder non-fluorescence quencher (MGB-NFQ) or ZEN-IB and Iowa Black Fluorescence quencher (IBFQ) at the 3′ end. In one embodiment the primer probe combination detects DNA encoding the structural capsid (VP) protein and/or the non-structural (NS) protein of Bovine parvovirus 3 in the test sample.

In one embodiment an internal positive control primer probe combination for detecting Bovine parvovirus 3 (BPV-3) genomic DNA in the extracted DNA of a test sample in combination with a primer probe combination for detecting Bovine parvovirus 3 genomic DNA, wherein the primer probe combination has the nucleic acid sequences of SEQ ID NO: 16, SEQ ID NO:17, and SEQ ID NO:18. In one embodiment SEQ ID NO: 18 has a fluorescent reporter dye and/or a non-fluorescent quencher. In one embodiment SEQ ID NO: 18 has a fluorescent reporter dye, 2′-chloro-7′phenyl-1,4-dichloro-6-carboxy-fluorescein (VIC) at the 5′ end and a Minor Groove Binder non-fluorescent quencher (MGB-NFQ) at the 3′ end.

Also provided is a primer probe combination for detecting Bovine parvovirus 3 genomic DNA in the extracted DNA of a test sample comprising SEQ ID NO:7, SEQ ID NO:8, and SEQ ID NO:9 in combination with an internal positive control primer probe combination comprising SEQ ID NO: 16, SEQ ID NO:17, and SEQ ID NO:18, wherein SEQ ID NO: 9 has a fluorescent reporter dye 6-carboxyfluorescein at the 5′ end and a Minor Groove Binder non-fluorescent quencher (MGB-NFQ) non-fluorescence quencher at the 3′ end and SEQ ID NO:18 has a fluorescent reporter dye, 2′-chloro-7′phenyl-1,4-dichloro-6-carboxy-fluorescein at the 5′ end and a Minor Groove Binder non-fluorescent quencher (MGB-NFQ) at the 3′ end.

The invention provides a method for determining the presence or absence of Bovine parvovirus 3 genomic DNA in the extracted DNA of a test sample. The method includes a reaction mixture comprising the extracted DNA of a test sample, nucleic acid amplification reagents, a primer probe combination selective for Bovine parvovirus 3 genomic DNA, and optionally an internal positive control primer probe combination; subjecting the reaction mixture to a real-time PCR technique (qPCR) to obtain copies of the target sequence, measuring any increase in fluorescence signal, wherein an increase in fluorescence signal indicates the presence of Bovine parvovirus 3 genomic DNA in the test sample. The reagents described herein can also be used for quantitative of the BPV-3 genome copy number in a test sample, for example, to determine the level of positivity, the viral load, in terms of viral genome quantity per ml of test sample.

The reaction mixture comprises the components to needed perform the quantitative PCR technique. Standard master mixes, component mixes and the like are commercially available, 2× TAQMAN® Universal PCR Master Mix (Applied Biosystems). The components typically include dNTPs (dATP, dCTP, dGTP, dTTP or dUTP), magnesium, TAQ DNA polymerase, buffers, and loading dyes if required by the PCR thermocycler being used. Others include Quantabio, PerfeCTa qPCR SuperMix, Low ROX (Quantabio, Beverly, MA). There are also a variety of commercially available thermocyclers. One of skill in the art would be able to determine which met their needs.

Real time PCR or qPCR is a technique that requires relatively small amounts of DNA, cDNA or RNA that can be quantified and facilitates monitoring the progress of a PCR in real time, as the reaction progresses. This PCR technique makes use of combinations of oligonucleotide primers and dual-labeled oligonucleotide probes. The probes act as a reporter, if amplified they accumulate with each cycle of the PCR reaction.

Specific detection of amplified product can be performed using one or more oligonucleotide probes that are labeled with a reporter fluorescent dye and a quencher dye. Such probes are known to those skilled in the art and are commercially available, including molecular beacons, dual-labeled probes, FRET (fluorescence resonance energy transfer) probes, and Scorpion ° probes. Oligonucleotide probes can be labeled with a reporter fluorescent dye and one or more quencher dyes. Examples of fluorescent reporter dyes include 6-carboxyfluorescein (FAM or 6-FAM), 2′-chloro-7′phenyl-1,4-dichloro-6-carboxy-fluorescein (VIC), TET™, HEX™, JOE, Cy® 3, CY® 5, CY® 5.5, TAMRA, ROX™, LC Red610, Texas Red®, LC640, SUN, MAX™, ATTO™ 550, ATTO 647™, Cal Fluor Gold 540 and Orange 560, TxRd (Sulforhodamine 101-X), Quasar 570 and 670. Examples of fluorescent quenchers include a Minor Groove Binder (MGB-NFQ), ZEN-IB, Black Hole Quencher® (BHQ 1, 2 and 3), TAMRA, Iowa Black® FQ and RQ.

For example, dual labeled probes may be labeled with one or more fluorescent reporter dyes at the 5′ end which fluoresce in presence of a complementary target and one or more non-fluorescence quenchers at the 3′end. The dual-labeled probes are designed to hybridize to the template between the two primers and are used in conjunction with a DNA polymerase enzyme that has inherent 5′ to 3′ endonuclease activity. When the probe is intact, the fluorescence of the reporter dye is quenched by the proximity of the quencher. During the extension phase of each PCR cycle, the 5′ exonuclease activity of DNA polymerase enzyme cleaves the annealed probe, releasing the reporter dye from the probe, resulting in an increase in fluorescence. This increase in fluorescence is directly proportional to the amount of amplified target DNA present in the reaction. The fluorescence is continually monitored throughout the PCR reaction. During the early cycles of the PCR reaction, the amount of fluorescence is below the detection threshold of the instrument. Monitoring for fluorescence signal continues, the first PCR cycle in which fluorescence is detected is noted. The more target DNA present in the sample at the outset of the reaction, the earlier fluorescence is detected, which reversely correlates with the sample target DNA quantity.

In one embodiment the primer probe combination selective for a DNA sequence of Bovine parvovirus 3 amplifies a 144 bp fragment. In one embodiment, the primer probe combination having the nucleic acid sequences of SEQ ID NO:7, SEQ ID NO:8, and SEQ ID NO:9 amplifies a 144 bp fragment.

The reaction mixture also includes a primer probe combination selective for a DNA sequence of Bovine parvovirus 3. In one embodiment the primer probe combination is selected from the group consisting of a) a primer probe combination having the nucleic acid sequences of SEQ ID NO:1, SEQ ID NO:2, and SEQ ID NO:3, wherein SEQ ID NO:3 has a fluorescent reporter dye at the 5′ end and a non-fluorescence quencher at the 3′ end; b) a primer probe combination having the nucleic acid sequences of SEQ ID NO:4, SEQ ID NO:5, and SEQ ID NO:6, wherein SEQ ID NO:6 has a fluorescent reporter dye at the 5′ end and a non-fluorescence quencher at the 3′ end; c) a primer probe combination having the nucleic acid sequences of SEQ ID NO:7, SEQ ID NO:8, and SEQ ID NO:9, wherein SEQ ID NO:9 has a fluorescent reporter dye at the 5′ end and a non-fluorescence quencher at the 3′ end; d) a primer probe combination having the nucleic acid sequences of SEQ ID NO:10, SEQ ID NO:11, and SEQ ID NO:12, wherein SEQ ID NO:12 has a fluorescent reporter dye at the end and a non-fluorescence quencher at the 3′ end; e) a primer probe combination having the nucleic acid sequences of SEQ ID NO:13, SEQ ID NO:14, and SEQ ID NO:15, wherein SEQ ID NO:15 has a fluorescent reporter dye at the 5′ end and a non-fluorescence quencher at the 3′ end; f) a primer probe combination having the nucleic acid sequences of SEQ ID NO:20, SEQ ID NO:21, and SEQ ID NO:22, wherein SEQ ID NO:22 has a fluorescent reporter dye at the 5′ end and a non-fluorescence quencher at the 3′ end; g) a primer probe combination having the nucleic acid sequences of SEQ ID NO:23, SEQ ID NO:24, and SEQ ID NO:25, wherein SEQ ID NO:25 has a fluorescent reporter dye at the 5′ end and a non-fluorescence quencher at the 3′ end; and f) a primer probe combination having the nucleic acid sequences of SEQ ID NO:26, SEQ ID NO:27, and SEQ ID NO:28, wherein SEQ ID NO:28 has a fluorescent reporter dye at the 5′ end and a non-fluorescence quencher at the 3′ end.

In one embodiment, the reaction mixture optionally includes an internal positive control primer probe combination. In one embodiment the mixture includes an internal positive control primer probe combination. In one embodiment the internal positive control primer probe combination has the nucleic acid sequences of SEQ ID NO: 16, SEQ ID NO:17, and SEQ ID NO:18, wherein SEQ ID NO:18 has a fluorescent reporter dye at the 5′ end and a non-fluorescence quencher at the 3′ end.

In one embodiment the method further comprising an internal positive control primer probe combination. In one embodiment the primer probe combination has the nucleic acid sequences of SEQ ID NO: 16, SEQ ID NO:17, and SEQ ID NO:18. In one embodiment SEQ ID NO: 18 has a fluorescent reporter dye and/or a non-fluorescent quencher. In one embodiment SEQ ID NO: 18 has a fluorescent reporter dye, 2′-chloro-7′phenyl-1,4-dichloro-6-carboxy-fluorescein (VIC) at the 5′ end and a Minor Groove Binder non-fluorescent quencher (MGB-NFQ) at the 3′ end.

In one embodiment one or more of SEQ ID NO: 3, SEQ ID NO:6, SEQ ID NO:9, SEQ ID NO:12, SEQ ID NO:15, SEQ ID NO:18, SEQ ID NO: 22, SEQ ID NO: 25, and SEQ ID NO: 28 have a fluorescent reporter dye selected from 6-carboxyfluorescein (FAM) or 2′-chloro-7′phenyl-1,4-dichloro-6-carboxy-fluorescein (VIC) at the 5′ end. In one embodiment SEQ ID NO: 3, SEQ ID NO:6, SEQ ID NO:9, SEQ ID NO:12, SEQ ID NO:15, SEQ ID NO: 22, SEQ ID NO: 25, and SEQ ID NO: 28 have a fluorescent reporter dye 6-carboxyfluorescein (FAM) at the 5′ end. In one embodiment, SEQ ID NO:18 has the fluorescent reporter dye 2′-chloro-7′phenyl-1,4-dichloro-6-carboxy-fluorescein (VIC) at the 5′ end.

In one embodiment one or more of SEQ ID NO:3, SEQ ID NO:6, SEQ ID NO:9, SEQ ID NO:12, SEQ ID NO:15, SEQ ID NO: 18, SEQ ID NO: 22, SEQ ID NO: 25, and SEQ ID NO: 28 have a non-fluorescent quencher at the 3′ end. In one embodiment the quencher is selected from a Minor Groove Binder non-fluorescent quencher (MGB-NFQ) and ZEN-IB and Iowa Black Fluorescence quencher (IBFQ) at the 3′ end.

In one embodiment one or more of SEQ ID NO:6, SEQ ID NO:9, SEQ ID NO:12, SEQ ID NO:15, and SEQ ID NO: 18 have a Minor Groove Binder non-fluorescent quencher (MGB-NFQ) at the 3′ end. In one embodiment one or more of SEQ ID NO: 3, SEQ ID NO: 22, SEQ ID NO: 25, and SEQ ID NO: 28 has ZEN-IB and Iowa Black Fluorescence quencher (IBFQ) at the 3′end.

In one embodiment SEQ ID NO: 3 has a fluorescent reporter dye 6-carboxyfluorescein (FAM) at the end and ZEN-IB and Iowa Black Fluorescence quencher (IBFQ) at the 3′ end. In one embodiment SEQ ID NO: 6 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 9 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 12 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 15 has a fluorescent reporter dye 6-carboxyfluorescein (FAM) at the 5′ end and a Minor Groove Binder non-fluorescent quencher (MGB-NFQ) at the 3′ end. In one embodiment SEQ ID NO: 18 has a fluorescent reporter dye 2′-chloro-7′phenyl-1,4-dichloro-6-carboxy-fluorescein (VIC) at the 5′ end and a Minor Groove Binder non-fluorescent quencher (MGB-NFQ) at the 3′ end. In one embodiment SEQ ID NO: 22 has a fluorescent reporter dye 6-carboxyfluorescein (FAM) at the 5′ end and ZEN-IB and Iowa Black Fluorescence quencher (IBFQ) at the 3′ end. In one embodiment SEQ ID NO: 25 has a fluorescent reporter dye 6-carboxyfluorescein (FAM) at the 5′ end and ZEN-IB and Iowa Black Fluorescence quencher (IBFQ) at the 3′ end. In one embodiment SEQ ID NO: 28 has a fluorescent reporter dye 6-carboxyfluorescein (FAM) at the 5′ end and ZEN-IB and Iowa Black Fluorescence quencher (IBFQ) at the 3′ end.

In one embodiment the primer probe combination targets the non-structural (NS) protein of BPV-3. In one embodiment the primer probe combinations target the non-structural (NS) protein and structural capsid (VP) protein of BPV-3. In one embodiment the primer probe combinations target the structural capsid (VP) protein of BPV-3.

In one embodiment the primer probe combinations target the structural capsid (VP) protein of BPV-3. In one embodiment, the primer probe combination comprises a forward primer that targets oligo position 3535-3554 of the BPV-3 isolate having the GenBank accession number AF406967, a reverse primer that targets oligo position 3667-3643 of the BPV-3 isolate having the GenBank accession number AF406967, and a probe that targets oligo position 3571-3588 of the BPV-3 isolate having the GenBank accession number AF406967. In one embodiment, in the primer probe combination the forward primer has the nucleic acid sequence of SEQ ID NO:1, the reverse primer has the nucleic acid sequence of SEQ ID NO:2, and the probe has the nucleic acid sequence of SEQ ID NO:3. In one embodiment, in the primer probe combination comprises a forward primer that targets oligo position 1734-1716 of the BPV-3 isolate having the GenBank accession number AF406967, a reverse primer that targets oligo position 1618-1645 of the BPV-3 isolate having the GenBank accession number AF406967, and a probe that targets oligo position 1665-1678 of the BPV-3 isolate having the GenBank accession number AF406967. In one embodiment, in the primer probe combination the forward primer has the nucleic acid sequence of SEQ ID NO:13, the reverse primer has the nucleic acid sequence of SEQ ID NO:14, and the probe has the nucleic acid sequence of SEQ ID NO:15. In one embodiment, in the primer probe combination comprises a forward primer that targets oligo position 2862-2878 of the BPV-3 isolate having the GenBank accession number AF406967, a reverse primer that targets oligo position 2963-2938 of the BPV-3 isolate having the GenBank accession number AF406967, and a probe that targets oligo position 2902-2930 of the BPV-3 isolate having the GenBank accession number AF406967. In one embodiment, in the primer probe combination the forward primer has the nucleic acid sequence of SEQ ID NO:20, the reverse primer has the nucleic acid sequence of SEQ ID NO:21, and the probe has the nucleic acid sequence of SEQ ID NO:22. In one embodiment, in the primer probe combination comprises a forward primer that targets oligo position 3051-3068 of the BPV-3 isolate having the GenBank accession number AF406967, a reverse primer that targets oligo position 3134-3114 of the BPV-3 isolate having the GenBank accession number AF406967, and a probe that targets oligo position 3079-3102 of the BPV-3 isolate having the GenBank accession number AF406967. In one embodiment, in the primer probe combination the forward primer has the nucleic acid sequence of SEQ ID NO:24, the reverse primer has the nucleic acid sequence of SEQ ID NO:25, and the probe has the nucleic acid sequence of SEQ ID NO:26. In one embodiment, in the primer probe combination comprises a forward primer that targets oligo position 3061-3079 of the BPV-3 isolate having the GenBank accession number AF406967, a reverse primer that targets oligo position 3140-3122 of the BPV-3 isolate having the GenBank accession number AF406967, and a probe that targets oligo position 3081-3103 of the BPV-3 isolate having the GenBank accession number AF406967. In one embodiment, in the primer probe combination the forward primer has the nucleic acid sequence of SEQ ID NO:26, the reverse primer has the nucleic acid sequence of SEQ ID NO:27, and the probe has the nucleic acid sequence of SEQ ID NO:28.

In one embodiment the primer probe combinations target the structural capsid (VP) protein and the non-structural (NS) protein of BPV-3. In one embodiment, the primer probe combination comprises a forward primer that targets oligo position 2190-2209 of the BPV-3 isolate having the GenBank accession number AF406967, a reverse primer that targets oligo position 2256-2236 of the BPV-3 isolate having the GenBank accession number AF406967, and a probe that targets oligo position 2213-2226 of the BPV-3 isolate having the GenBank accession number AF406967. In one embodiment the forward primer has the nucleic acid sequence of SEQ ID NO:4, the reverse primer has the nucleic acid sequence of SEQ ID NO:5, and the probe has the nucleic acid sequence of SEQ ID NO:6.

In one embodiment the primer probe combination targets the non-structural (NS) protein of BPV-3. In one embodiment, the primer probe combination comprises a forward primer that targets oligo position 1331-1352 of the BPV-3 isolate having the GenBank accession number AF406967, a reverse primer that targets oligo position 1474-1453 of the BPV-3 isolate having the GenBank accession number AF406967, and a probe that targets oligo position 1377-1391 of the BPV-3 isolate having the GenBank accession number AF406967. In one embodiment the forward primer has the nucleic acid sequence of SEQ ID NO:7, the reverse primer has the nucleic acid sequence of SEQ ID NO:8, and the probe has the nucleic acid sequence of SEQ ID NO:9. In one embodiment, the primer probe combination comprises a forward primer that targets oligo position 1453-1474 of the BPV-3 isolate having the GenBank accession number AF406967, a reverse primer that targets oligo position 1562-1539 of the BPV-3 isolate having the GenBank accession number AF406967, and a probe that targets oligo position 1507-1522 of the BPV-3 isolate having the GenBank accession number AF406967. In one embodiment the forward primer has the nucleic acid sequence of SEQ ID NO:10, the reverse primer has the nucleic acid sequence of SEQ ID NO:11, and the probe has the nucleic acid sequence of SEQ ID NO:12.

In one embodiment the primer probe combination comprises SEQ ID NO:7, SEQ ID NO:8, and SEQ ID NO:9 in combination with an internal positive control primer probe combination comprising SEQ ID NO: 16, SEQ ID NO:17, and SEQ ID NO:18, wherein SEQ ID NO: 9 has a fluorescent reporter dye 6-carboxyfluorescein at the 5′ end and a Minor Groove Binder non-fluorescent quencher (MGB-NFQ) non-fluorescence quencher at the 3′ end and SEQ ID NO:18 has a fluorescent reporter dye, 2′-chloro-7′phenyl-1,4-dichloro-6-carboxy-fluorescein at the 5′ end and a Minor Groove Binder non-fluorescent quencher (MGB-NFQ) at the 3′ end.

The reaction mixtures used in the methods described herein may further comprise one or more controls. These controls include one or more of a no template control that contains all the PCR reagents except for the DNA template; a negative extraction control to monitor cross contamination during nucleic acid extraction and verify that the assay does not produce false positive results with non-specific background DNA; a positive extraction control to evaluate DNA extraction performance during test sample preparation and verify that the assay does not produce false negative results; a positive control to verify that the qPCR components work accurately and give a PCR amplification signal specific to the target sequence; an inhibition control to determined sample interference/inhibition level, a standard to determine the PCR efficiency, linear range and to quantify the absolute copy number of target sequence in the extracted DN of the test sample/control, and/or internal positive control to monitor cross contamination of positive control plasmid in the test sample that is used during sample preparation and assay execution.

A suitable level of precision, accuracy and linearity of the assay is preferentially demonstrated within the dynamic range of the analytical assays described herein, particularly for quantitative assays. The precision of the assay is based on repeatability (intra-assay precision) and intermediate precision (within-laboratory precision). Repeatability is the coefficient of variation (CV) of the results obtained with the same method by the same analyst, in the same laboratory, with the same equipment, on the same samples over a short period of time. To determine the repeatability of the assay the mean and standard deviation (StdDev) of quantity (genome copy of target sequence per reaction, GC/rxn) and % CV of quantity is calculated from each set of PCR reactions at respected concentration. Intermediate precision accounts for the inherent variability, such as different analysts, different detection systems, different times. To determine intermediate precision of the assay the mean and StdDev of quantity and % CV of quantity is calculated from all sets of PCR reactions of the all respected concentrations of the independent experiments over the time.

Accuracy (also referred to as trueness) compares the obtained value from a series of samples (such as a positive control plasmid with defined concentrations) to the actual or reference value, called the standard (ST). To determine accuracy of the assay the mean of quantity (genome copy of target sequence per reaction, GC/rxn) is calculated from each set of PCR reactions at the respected concentration.

The limit of quantification (LOQ) of a quantitative qPCR assay is the lowest amount of target sequence in a sample which can be quantitatively determined with suitable precision (repeatability and intermediate precision) and accuracy. To determine the LOQ, the lowest, highest, and some number of middle ranges are tested. The lowest, middle and upper range concentration of positive control plasmid is prepared and spiked into an exogenous extracted DNA and quantified in an appropriate number of replicates in the presence of standard curve preferably using different analysts performing the assay on different dates.

The limit of detection (LOD) or sensitivity of the assay is the lowest amount of target sequence which can be detected by the assay, but not necessarily quantitated as an exact value.

The robustness of the assay may be determined according to the method of Youden and Steiner (Youden, Steiner, Statistical Manual of the Association of Official Analytical Chemists, Association of Official Analytical Chemists ed., Arlington 1975, pps. 33-36, 70-71, 82083). For example, changes to certain critical reagent concentrations may be evaluated. Certain critical PCR factors may be selected and subjected to slight changes. The acceptance criterion would require that the response obtained for any robustness condition with respect to the applied small changes should meet established assay acceptance criteria.

The invention provides an assay for the quantification of Bovine parvovirus 3 genomic DNA in the extracted DNA of a test sample. In one embodiment is provided an assay for the quantification of 1E3 to 1E8 genome copies of Bovine parvovirus 3 genomic DNA in a PCR reaction.

The invention provides a method for the quantification of 1E3 to 1E8 genome copies of Bovine parvovirus 3 genomic DNA in PCR reaction comprising 1) a reaction mixture comprising the extracted DNA of a test sample, a positive control, a BPV-3 IPC positive control plasmid DNA, nucleic acid amplification reagents, an internal positive control primer probe combination having the nucleic acid sequences of SEQ ID NO: 16, SEQ ID NO:17, and SEQ ID NO:18, wherein SEQ ID NO:18 has a fluorescent reporter dye at the 5′ end and a non-fluorescence quencher at the 3′ end, and a primer probe combination selective for a DNA sequence of Bovine parvovirus 3, selected from the group consisting of a) a primer probe combination having the nucleic acid sequences of SEQ ID NO:1, SEQ ID NO:2, and SEQ ID NO:3, wherein SEQ ID NO:3 has a fluorescent reporter dye at the 5′ end and a non-fluorescence quencher at the 3′ end; b) a primer probe combination having the nucleic acid sequences of SEQ ID NO:4, SEQ ID NO:5, and SEQ ID NO:6, wherein SEQ ID NO:6 has a fluorescent reporter dye at the 5′ end and a non-fluorescence quencher at the 3′ end; c) a primer probe combination having the nucleic acid sequences of SEQ ID NO:7, SEQ ID NO:8, and SEQ ID NO:9, wherein SEQ ID NO:9 has a fluorescent reporter dye at the 5′ end and a non-fluorescence quencher at the 3′ end; d) a primer probe combination having the nucleic acid sequences of SEQ ID NO:10, SEQ ID NO:11, and SEQ ID NO:12, wherein SEQ ID NO:12 has a fluorescent reporter dye at the 5′ end and a non-fluorescence quencher at the 3′ end; e) a primer probe combination having the nucleic acid sequences of SEQ ID NO:13, SEQ ID NO:14, and SEQ ID NO:15, wherein SEQ ID NO:15 has a fluorescent reporter dye at the 5′ end and a non-fluorescence quencher at the 3′ end; f) a primer probe combination having the nucleic acid sequences of SEQ ID NO:13, SEQ ID NO:20, and SEQ ID NO:21, wherein SEQ ID NO:21 has a fluorescent reporter dye at the 5′ end and a non-fluorescence quencher at the 3′ end; g) a primer probe combination having the nucleic acid sequences of SEQ ID NO:23, SEQ ID NO:24, and SEQ ID NO:25, wherein SEQ ID NO:25 has a fluorescent reporter dye at the 5′ end and a non-fluorescence quencher at the 3′ end; and h) a primer probe combination having the nucleic acid sequences of SEQ ID NO:26, SEQ ID NO:27, and SEQ ID NO:28, wherein SEQ ID NO:28 has a fluorescent reporter dye at the 5′ end and a non-fluorescence quencher at the 3′ end; 2) subjecting the reaction mixture to a quantitative PCR technique to obtain copies of the target sequence, and 3) measuring any increase in fluorescence signal.

In one embodiment the dynamic range is 1E3 to 1E8 GC/rxn. In one embodiment the lower limit of quantification is 1E3 GC/rxn. In one embodiment the upper limit of quantification is 1E8 GC/rxn. In one embodiment the limit of detection (LOD95%) of the quantification assay is 27 genome copies per reaction with a 95% confidence interval of 22 and 34 genome copies per reaction. In one embodiment the assay has linearity having a correlation coefficient (R2)≥0.98 and a PCR amplification efficiency within 90-110%. In one embodiment the assay has a repeatability value that is a % CV of quantity equal or less than 25%. In one embodiment the assay has an intermediate precision value that is % CV of quantity equal or less than 30%. In one embodiment the assay has an accuracy value within ±30% of the accepted reference value (ST) across the whole dynamic range of the assay. In one embodiment the assay has a limit of quantitation that is the % CV of quantity for repeatability at ≤25%, intermediate precision at ≤30% and acceptance criterion for the accuracy (the value for lowest, middle and upper ranges) within ±30% of the expected standard (ST) reference value. In one embodiment the assay has a robustness that has a percent CV of quantity for repeatability of ≤25%, intermediate precision of ≤30% and accuracy of the mean of quantity of the combination matrix condition tested of ±30% of the mean of quantity of the optimized condition.

The invention provides a kit for use in detecting Bovine parvovirus 3 (BPV-3) genomic DNA contamination in the extracted DNA of a test sample comprising a primer probe combination that detects DNA encoding BPV-3 selected from the group consisting of a) a primer probe combination having the nucleic acid sequences of SEQ ID NO:1, SEQ ID NO:2, and SEQ ID NO:3; b) a primer probe combination having the nucleic acid sequences of SEQ ID NO:4, SEQ ID NO:5, and SEQ ID NO:6; c) a primer probe combination having the nucleic acid sequences of SEQ ID NO:7, SEQ ID NO:8, and SEQ ID NO:9; d) a primer probe combination having the nucleic acid sequences of SEQ ID NO:10, SEQ ID NO:11, and SEQ ID NO:12; e) a primer probe combination having the nucleic acid sequences of SEQ ID NO:13, SEQ ID NO:14, and SEQ ID NO:15; f) a primer probe combination having the nucleic acid sequences of SEQ ID NO:20, SEQ ID NO:21, and SEQ ID NO:22; g) a primer probe combination having the nucleic acid sequences of SEQ ID NO:23, SEQ ID NO:24, and SEQ ID NO:25; and g) a primer probe combination having the nucleic acid sequences of SEQ ID NO:26, SEQ ID NO:27, and SEQ ID NO:28; and optionally an internal positive control primer probe combination having the nucleic acid sequences of SEQ ID NO: 16, SEQ ID NO:17, and SEQ ID NO:18.

While the terminology used in this application is standard within the art, definitions of certain terms are provided herein to assure clarity and definiteness to the meaning of the claims. Units, prefixes, and symbols may be denoted in their International System of Units (SI) accepted form. Numeric ranges recited herein are inclusive of the numbers defining the range and include and are supportive of each integer within the defined range. The methods and techniques described herein are generally performed according to conventional methods well known in the art and as described in various general and more specific references that are cited and discussed throughout the present specification unless otherwise indicated. All documents, or portions of documents, cited in this application, including but not limited to patents, patent applications, articles, books, and treatises, are hereby expressly incorporated by reference.

The present invention is not to be limited in scope by the specific embodiments described herein that are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. What is described in an aspect or embodiment of the invention can be combined with other aspects and/or embodiments of the invention.

The following examples, including the experiments conducted and the results achieved, are provided for illustrative purposes only and are not to be construed as limiting the scope of the appended claims.

### EXAMPLES

### Example 1 BPV-3 Primer/Probe Set Development

To locate conserved regions on the BPV-3 genome, all published full-length genome sequence of BPV-3 isolates were retrieved from GenBank (Table 1).

TABLE 1
GenBank Accession numbers.

| | | | GenBank |
|---|---|---|---|
| Genus | Species | Isolate | Accession # |
| Erythroparvovirus | Ungulate | Bovine | AF406967 |
| | erythroparvovirus 1 | parvovirus 3 | MG745680 |
| | | | NC_037053 |
| | | | MG026727 |
| | | | MG026728 |

Multiple alignments were performed and different conserved regions in the genes encoding the BPV-3 non-structural (NS) protein and structural capsid (VP) protein were selected and used to design eight different primer/probe sets (BPV-3 v1.0-BPV-3 v8.0). The probes were labeled with a fluorescent reporter dye 6-carboxyfluorescein (FAM) at the 5′ end and either a Minor Groove Binder, non-fluorescence quencher (MGB-NFQ) or ZEN-IB and Iowa Black Fluorescence quencher (IBFQ) at the 3′ end, (Applied Biosystems, Carlsbad, CA or Integrated DNA Technologies, Inc., Coralville, IA). Table 2 provides the oligo positions and sequences of the probes and primer sets. The primer/probe sets were synthesized and tested using five-log concentration range of positive control plasmid (1e7 to 1e3) with 6 replicates.

TABLE 2
Prime and probe sequence used for BPV-3 v3.0. Oligonucleotide position for the
probe/primers is based on BPV-3 isolate GenBank accession number AF406967.

| Probe | | | |
|---|---|---|---|
| Primer | Oligo | | |
| Target | position | Oligo function | Sequence (5′→3′) |
| BPV-3 | 3535-3554 | Forward | CATCGGCTATAAAACCCCAT |
| v1.0 | | Primer | SEQ ID NO: 1 |
| Gene: | 3667-3643 | Reverse | GGACAGTATTATTTCCATGCTCTCT |
| VP | | Primer | SEQ ID NO: 2 |
| | 3571-3588 | Probe | CAACGCCATCAATCGCCA |
| | | | SEQ ID NO: 3 |
| | | | Reporter Dye: FAM |
| | | | Quencher Dye: ZEN AND IBFQ |
| BPV-3 | 2190-2209 | Forward | GGGTTAGAAACGCTGCAATC |
| v2.0 | | Primer | SEQ ID NO: 4 |
| Gene: | 2256-2236 | Reverse | AGCAAGCGAGAGGACATAGTC |
| NS-VP | | Primer | SEQ ID NO: 5 |
| | 2213-2226 | Probe | TTAGGTATGGCGTT |
| | | | SEQ ID NO: 6 |
| | | | Reporter Dye: FAM |
| | | | Quencher Dye: MGB-NFQ |
| BPV-3 | 1331-1352 | Forward | GCATAAATGTGTCTTGGTGTGG |
| v3.0 | | Primer | SEQ ID NO: 7 |
| Gene: | 1474-1453 | Reverse | ATTAATACGGGAGTGGGAGACA |
| NS | | Primer | SEQ ID NO: 8 |
| | 1377-1391 | Probe | ATTGTTGAGGCTGTA |
| | | | SEQ ID NO: 9 |
| | | | Reporter Dye: FAM |
| | | | Quencher Dye: MGB-NFQ |
| BPV-3 | 1453-1474 | Forward | TGTCTCCCACTCCCGTATTAAT |
| v4.0 | | Primer | SEQ ID NO: 10 |
| Gene: | 1562-1539 | Reverse | AATGACCATCCTCTCACTTAAAGC |
| NS | | Primer | SEQ ID NO: 11 |
| | 1507-1522 | Probe | ATGGAAACATTGTTAC |
| | | | SEQ ID NO: 12 |
| | | | Reporter Dye: FAM |
| | | | Quencher Dye: MGB-NFQ |
| BPV-3 | 1618-1645 | Forward | TTGTTGATTGGCTAAACTATGTAAAGTC |
| v5.0 | | Primer | SEQ ID NO: 13 |
| Gene: | 1734-1716 | Reverse | GGGCTTTCCGCTTTATCTC |
| VP | | Primer | SEQ ID NO: 14 |
| | 1665-1678 | Probe | GCTGATACCGTTCA |
| | | | SEQ ID NO: 15 |
| | | | Reporter Dye: FAM |
| | | | Quencher Dye: MGB-NFQ |
| BPV-3 | 2862-2878 | Forward | ATGCCTCCGAGCAACAC |
| v6.0 | | Primer | SEQ ID NO: 20 |
| Gene: | 2963-2938 | Reverse | CCTCTCTAGACTCTTCTCTAGATTCA |
| VP | | Primer | SEQ ID NO: 21 |
| | 2902-2930 | Probe | CCCAGATATCACATCTTCTGTCGGAAACA |
| | | | SEQ ID NO: 22 |
| | | | Reporter Dye: FAM |
| | | | Quencher Dye: ZEN AND IBFQ |
| BPV-3 | 3051-3068 | Forward | CGGTGGCCACAGCATATC |
| v7.0 | | Primer | SEQ ID NO: 23 |
| Gene: | 3134-3114 | Reverse | GGGACCCACTAGGATACATTTG |
| VP | | Primer | SEQ ID NO: 24 |
| | 3079-3102 | Probe | TCAGTTCTTGCGGGACTACTTGGC |
| | | | SEQ ID NO: 25 |
| | | | Reporter Dye: FAM |
| | | | Quencher Dye: ZEN AND IBFQ |
| BPV-3 | 3061-3079 | Forward | AGCATATCCTCGGCCTGAT |
| v8.0 | | Primer | SEQ ID NO: 26 |
| Gene: | 3140-3122 | Reverse | GATACCGGGACCCACTAGG |
| VP | | Primer | SEQ ID NO: 27 |
| | 3081-3103 | Probe | AGTTCTTGCGGGACTACTTGGCC |
| | | | SEQ ID NO: 28 |
| | | | Reporter Dye: FAM |
| | | | Quencher Dye: ZEN AND IBFQ |

Based on LOD, dynamic range, PCR efficiency and R² value, BPV-3 v3.0 was selected for use in assay development and qualification. The primers amplified a 144-bp fragment of BPV 3. The probe has a fluorescent reporter dye 6-carboxyfluorescein (FAM) at the 5′ end and a Minor Groove Binder, non-fluorescence quencher (MGB-NFQ) at the 3′ end, (Applied Biosystems, Carlsbad, CA). The designed BPV-3 v3.0 primer/probe sequences fully matched with the sequences of all five of the Parvovirinae subfamily sequences by in silico analysis. Multiple alignments with other parvoviruses did not produce a match indicating the sequences of the primer probe combination were specific for BPV-3.

### Internal Positive Control Primer/Probe Set and BPV-3_IPC Positive Control Plasmid DNA

An internal positive control (IPC) was designed to monitor the extraction efficiency and any accidental cross-contamination between spiked positive control plasmids and the extracted DNA of the test sample. The IPC probe (IPC v2.0) was labeled with a fluorescent reporter dye, 2′-chloro-7′phenyl-1,4-dichloro-6-carboxy-fluorescein (VIC), at the 5′ end and a Minor Groove Binder non-fluorescent quencher (MGB-NFQ) at the 3′ end (Applied Biosystems, Carlsbad, CA). The IPC primer/probe set amplified a 67-bp fragment, to facilitate distinguishing it from the BPV-3 primer/probe. Table 3 provides the oligo function and sequences of the probes and primer. This probe/primer set was used for further BPV-3 assay development and qualification.

TABLE 3
Prime and probe sequence used for IPC v2.0.

| Probe Primer | | |
|---|---|---|
| Target | Oligo function | Sequence (5′→3′) |
| IPC v2.0 | Forward Primer | AACTTGGTCAGAGATCGAGGAGG SEQ ID NO: 16 |
| | Reverse Primer | CCGCCAGTGTGCTGGAAT SEQ ID NO: 17 |
| | Probe | CGCCCTTCGAAGCAC SEQ ID NO: 18 |
| | | Reporter Dye: VIC-MGB |
| | | Quencher Dye: MGB-FNQ |

To generate BPV-3_IPC positive control plasmid DNA, the target sequences of the BPV-3 and IPC were synthesized and cloned into a pUC57 vector. The 5′ termini of the positive control sequence carried the BPV-3 target genome and 3′ termini and the positive control sequence carried IPC target sequence. Therefore, the positive control plasmid carries both BPV-3 and IPC sequence, which served as an BPV-3 positive control as well as internal positive control to monitor extraction efficiency and any accidental cross contamination. The sequence of the positive control DNA is shown in Table 4.

TABLE 4
BPV-3_IPC positive control plasmid DNA.

5′AATGAAAATTTTCCCTTCAATGATGCGCCGCATAAATGTGTCTTGGT
GTGGGACGAAGGCCGAATTACGGCCAAAATTGTTGAGGCTGTAAAGAGC
ATTCTGGGGGGTCAGGCGGTGCGGGTGGATCAGAAATGCAAGGGCTCTG
TAAGCTTGTCTCCCACTCCCGTATTAATCACATCTAATGCTGATATTCG
ATATGTGCGTGATGGAAACATTGTTACTGGGGATCATGTAAAAGCTTTA
AGTGAGAGGATGGTCATTGTACATTTCTCTACTCCGTGCCCCGCCAATT
TTGGGCTTTTGAAAGCGGAGGAAATTGTTGATTGGCTAAACTATGTAAA
GTCATGCCCGGGGAGTATCACTGCTGATACCGTTCAGGCCACGTGGGGA
ACACGCTCCGCCCCCAATTTATTTGAGATAAAGCGGAAAGCCCCACAGC
CCGCCAGTCCCATTGAACCTCAGACGGAGGAACAAGAAGAAGCAGCCGC
ATATCGCTGTCAGAGCCCAACTTGGTCAGAGATCGAGGAGGATTTGAGA
GCGTGCTTCGAAGGGCGAATTCCAGCACACTGGCGGCCGTTACTGCGCG
CACATTCACCAAG 3′ SEQ ID NO: 19

### Example 2 BPV-3 Qualitative Assay

### Sample Preparation

For test samples that contained cells, the samples were subjected to a low speed centrifugation (LSC) before DNA extraction, 320×g to 1000×g, for 10 minutes at room temperature. The supernatant was collected for DNA extraction. Cell free samples were processed directly.

To a 250 μL of a test sample was added, 10 μL of an RNase Cocktail™ Enzyme Mix (Thermo Fisher, Carlsbad, CA) and 0.5M EDTA, pH 8.0 (9 μL:1 μL) mixture. An AutoMate Express™ Nucleic Acid Extraction System (ThermoFisher) was used to extract the DNA from the test sample. On the AutoMate, the PrepSEQ (PS) Express 1-2-3 protocol was selected for the DNA extraction procedure with following parameters: 1-hour proteinase K (PK) digestion and 100 μL elution.

Extracted DNA was visually inspected and if there were any residual magnetic beads in the eluted DNA, a DynaMag™-2 Magnet benchtop workstation (ThermoFisher) was used the remove any remaining beads in the eluted DNA. The DNA was used immediately or stored at −30 C.

Alongside the DNA extracted from the test samples, a BPV-3 IPC (PEC) plasmid control and a negative extraction control (NEC) were also prepared and run as described above. For the positive extraction control, 250 μL sterile 1× phosphate buffered saline (PBS) was spiked with 62,000 genome copies (GC) BPV-3_IPC plasmid DNA. The positive extraction control was used to evaluate DNA extraction efficiency during sample preparation. Assuming 100% efficiency of DNA extraction, final eluted DNA should contain 500 GC/μL. The negative extraction control contained 250 μL sterile 1× PBS. The negative extraction control was used to monitor any accidental cross contamination that may happen during nucleic acid extraction.

Preparation of additional BPV-3 detection assay controls No template control (NTC): The no template control contained all the PCR reagents except for the DNA template. This was used as a negative control to verify qPCR raw materials were free of any DNA contamination.

Positive control (PC): the positive control contained 2000 GC/reaction of BPV-3_IPC recombinant plasmid DNA. The positive control was used to verify the accuracy of the assay.

Inhibition control (IHN): The inhibition control is used to monitor PCR amplification and the level of interference or inhibition imposed from the test sample matrix. The final extracted DNA from the test sample was spiked (+S) with same amount of BPV-3_IPC plasmid (2000 GC/reaction) as the positive control. The CT (cycle threshold) is the number of cycles required for the fluorescent signal to cross the threshold (i.e. exceeds background level). The mean cycle threshold (CT) value of the inhibition control (+S) was compared with the mean CT value of the positive control as a measure of interference or inhibition. The ΔCT value should be less than 3.32 CT (equal to one log of DNA concentration). The inhibition or interference was calculated as ΔCT=|PC (mean CT)−IHN (mean CT).

BPV-3 qPCR and IPC qPCR Detection Assay Setups

Two different master mixes are prepared, a BPV-3 qPCR master mix for the BPV_3 pPCR assay and an IPC qPCR master mix and the IPC qPCR assay.

The BPV-3 qPCR detection assay setup contained the BPV-3 v3.0 prime/probe set to detect BPV-3 DNA in the DNA extracted from a test sample. The BPV-3 qPCR master mix comprised the BPV-3 v3.0 Primer-Probe set, water, and 2× TAQMAN® Universal PCR Master Mix (Applied Biosystems). The 2× TAQMAN® Universal PCR Master Mix contained the essential components for the qPCR reaction including optimized buffers to amplify G/C-rich sequence, dNTPs with dUTP, AmpliTaq Gold™ DNA polymerase, ROX dye (as a passive internal reference) and AmpErase™ UNG (Uracil-DNA Glycosylase enzyme to eliminate carry-over contamination PCR products containing dU).

Table 5 provides the amount of each reaction component for each sample tested in the BPV-3 qPCR reaction: the DNA extracted from the test sample and the five controls: negative extraction control (NEC), no template control (NTC), positive extraction control (PEC), inhibition control (IHN), and positive control (PC) at two different concentrations, an above detection limit (ADL) amount and a detection limit (DL) amount.

TABLE 5
With inhibition positive controls tested at and above the detection limit

| | | 2X TAQMAN ® | | Extracted | BPV_IPC PC | |
|---|---|---|---|---|---|---|
| | | Universal | BPV-3 v3.0 | DNA (μL) | Plasmid | |
| | Water | PCR Master | Primer-Probe | from test | (GC/μL) | |
| | (μL) | Mix (μL) | 60x (μL) | sample | 125 | 12.5 |
| Test Sample | 4.5 | 15 | 0.5 | 10 | n/a | n/a |

Controls

| | | | | | | |
|---|---|---|---|---|---|---|
| NEC | 4.5 | 15 | 0.5 | 10 | n/a | n/a |
| NTC | 14.5 | 15 | 0.5 | n/a | n/a | n/a |
| PEC | 4.5 | 15 | 0.5 | 10 | n/a | n/a |
| IHN_ADL | 2.5 | 15 | 0.5 | 10 | 2 | n/a |
| (250 GC/RXN) | | | | | | |
| IHN_DL | 2.5 | 15 | 0.5 | 10 | n/a | 2 |
| (25 GC/RXN) | | | | | | |
| PC_ADL | 12.5 | 15 | 0.5 | n/a | 2 | n/a |
| (250 GC/RXN) | | | | | | |
| PC_DL | 12.5 | 15 | 0.5 | n/a | n/a | 2 |
| (25 GC/RXN) | | | | | | |

For the IPC qPCR reaction an IPC prime/probe master mix was prepared that comprised the IPC Primer-Probe, water, and 2× TAQMAN® Universal PCR Master Mix (Applied Biosystems). The amount of each reaction component for the test sample as well as for the four controls, negative extraction control, no template control, positive extraction control, and positive control at two different concentrations, an above detection limit (ADL) amount and a detection limit (DL) amount are provided in Table 6.

TABLE 6
IPC qPCR reaction component (qualitative assay)

| | | 2X TAQMAN ® | | Extracted | BPV_IPC PC | |
|---|---|---|---|---|---|---|
| | | Universal | IPC v2.0 | DNA (μL) | Plasmid | |
| | Water | PCR Master | Primer-Probe | from test | (GC/μL) | |
| | (μL) | Mix (μL) | 60x (μL) | sample | 125 | 12.5 |
| Test Sample | 4.5 | 15 | 0.5 | 10 | n/a | n/a |

Controls

| | | | | | | |
|---|---|---|---|---|---|---|
| NEC | 4.5 | 15 | 0.5 | 10 | n/a | n/a |
| NTC | 14.5 | 15 | 0.5 | n/a | n/a | n/a |
| PEC | 4.5 | 15 | 0.5 | 10 | n/a | n/a |
| PC_ADL | 12.5 | 15 | 0.5 | n/a | 2 | n/a |
| (250 GC/RXN) | | | | | | |
| PC_DL | 12.5 | 15 | 0.5 | n/a | n/a | 2 |
| (25 GC/RXN) | | | | | | |

Both the BPV_3 qPCR reaction and the IPC qPCR reaction were executed at least in triplicate (3 PCR reaction wells) for each sample and control in a 96 well reaction plate. The reaction components could be set up either manually or using a liquid handler robot, such as the QIAgility HEPA/UV liquid handler (QIAGEN, Inc., Germantown, MD). A MicroAmp Optical 96-well plate (Applied Biosystems) was used to accommodate the qPCR reaction mixtures and covered with MicroAmp Optical Adhesive Film (Applied Biosystems). The amount of PCR reagent components, samples, and controls in each PCR reaction is listed in Tables 6 and 7. The sealed plate was then loaded into a QuantStudior™ 7 Flex Real-Time PCR System (Applied Biosystems).

A QuantStudior™ 7 Flex Real-Time PCR System (Applied Biosystems) was used to perform the qPCR reactions. The thermal cycling program was set for the following reaction times and temperatures: 50° C. 2 min, 95° C. 10 min, and 40 cycle of 95° C. 15 sec, 60° C. 1 min (data collection). Table 7 provides the experimental properties that were selected.

TABLE 7
QuantStudio ™ software experimental
properties for BPV-3 qPCR and IPC qPCR.

| Experimental property name: | Experimental property selected: |
|---|---|
| Instrument | QuantStudio ™ 7 Flex System |
| Block | 96-Well (0.2 mL) |
| Experiment | Standard Curve |
| Reagent | TAQMAN ® Reagents |
| Run Properties | Standard |
| Reporter | FAM (for BPV-3 v3.0 primer/probe set) |
| | VIC (for IPC v2.0 primer/probe set) |
| Quencher | MGB-NFQ |
| Passive Dye | ROX |
| Threshold | ΔRn 0.1 for BPV-3 assay |
| | ΔRn 0.2 for IPC assay |
| Baseline | Automatic |
| Reaction volume | 30 μL |

Before test samples are evaluated, the assay must have met system suitability and assay acceptance criteria to be considered a valid assay. If the assay acceptance criteria are not met, the assay must be repeated. If the test sample was valid and positive, the assay should be repeated to confirm the achieved positive result. The criteria for a valid assay and the evaluation of the test results for the BPV-3 qualitative assay is outlined in Table 8. If the test sample presents a positive amplification signal, the IHN and ΔCT controls are irrelevant and should not be considered as a part of system suitability and assay acceptance criteria.

TABLE 8
System suitability and assay acceptance criteria.

| | |
|---|---|
| Target BPV-3 | At least 2 out of 3 reactions of the NTC and NEC must |
| and IPC for | show no amplification signal (CT = 40.00). |
| NTC and NEC | |
| Target BPV-3 | The PC, PEC and IHN must produce amplification signal |
| for PC, PEC, | in at least 2 out of 3 reactions (CT less than 40.00). |
| and IHN | |
| Target IPC for | The PC and PEC must produce amplification in at least |
| PC and PEC | 2 out of 3 reactions (CT less than 40.00). |
| Target BPV-3 | Calculate interference/inhibition per ΔCT = \| PC (mean |
| for ΔCT | CT) − IHN (mean CT) \| equation. If the ΔCT is equal |
| | and greater than 3.32, the assay is invalid. |

### Test Sample Results Acceptance Criteria

If the assay meets the system suitability and assay acceptance criteria, Table 9 provides the test sample acceptance criteria. The results of the qualitative assay are reported as “positive” or “negative”.

TABLE 9
Test sample acceptance criteria.

| | |
|---|---|
| Positive | A test sample is reported as “positive” if at least 2 out of 3 |
| test | reactions present a positive amplification signal (CT less than |
| sample | 40.00) for target BPV-3; and at least 2 out of 3 replicates |
| criteria | present no amplification signal (CT = 40.00) for target IPC. |
| Negative | A test sample is reported as “negative” if at least 2 out of 3 |
| test | replicates show no amplification signal (CT = 40.00) for both |
| sample | targets BPV-3 and IPC. |
| criteria | |

### Repeating Testing Strategy

The assay may be repeated using 1:2 dilution of extracted DNA if ΔCT does not meet the acceptance criteria using neat DNA sample. The dilution will reduce sample matrix interference. In this case all samples and control DNA must be diluted as 1:2 with molecular biology grade water before repeating the assay.

If a test sample shows positive amplification signal for the target IPC, this indicates cross contamination of positive control plasmid DNA with test sample. In this case the assay is invalid and after implementation of decontamination per a site applicable procedure, the assay needs to be repeated.

### Example 3 Testing the BPV3 Qualitative Assay

### Specificity: Sample Matrix Effect (to Test for the Absence of False Positives)

Three sample matrices (Dulbecco's Modified Eagle Medium (DMEM) (ThermoFisher) and two fetal bovine serum (FBS) (SAFC St. Louis, MO, and Hyclone Logan, UT) were tested to demonstrate the absence of false positive results between media components and the BPV-3 v3.0 and IPC v2.0 primer/probe sets. The assay procedures and set up was as described in Example 2.

Table 10 shows the tested samples and the results of qPCR testing with the BPV-3 and IPC assays. The acceptance criterion for this procedure required that all tested samples show a negative result with both primer/probe sets. Results from valid experiments when all implemented controls and system suitability met acceptance criteria revealed that all tested samples were negative and showed no amplification signal (CT=40.00).

TABLE 10
Results from the sample matrices.

| Sample | PCR | BPV-3 v3.0 | IPC v2.0 | |
|---|---|---|---|---|
| matrix | Rep. | Mean ± SD CT | Mean ± SD CT | Results |
| DMEM | 6 | 40.00 ± 0.00 | 40.00 ± 0.00 | NEG/Pass |
| DMEM | 6 | 40.00 ± 0.00 | 40.00 ± 0.00 | NEG/Pass |
| FBS | 6 | 40.00 ± 0.00 | 40.00 ± 0.00 | NEG/Pass |
| FBS | 6 | 40.00 ± 0.00 | 40.00 ± 0.00 | NEG/Pass |
| NTC | 3 | 40.00 ± 0.00 | 40.00 ± 0.00 | NEG/Pass |
| PC | 3 | 29.21 ± 0.26 | 28.67 ± 0.04 | POS/Pass |
| PEC | 3 | 28.57 ± 0.13 | 28.10 ± 0.21 | POS/Pass |

### BPV-3 Detection (to Test the Absence of False Negatives)

Due to lack of a commercially available BPV-3 virus stock or BPV-3 genomic DNA, the BPV-3_IPC positive control plasmid was used to test the specificity of the BPV-3 v3.0 primer/probe set. The BPV-3_IPC plasmid was sequenced, and the accuracy of the BPV-3 NS gene was confirmed by BLAST analysis.

The assay as described in Example 2 was used. As part of specificity testing, two fetal bovine serum samples and an unprocessed non-GMP bulk harvest sample containing FBS which were confirmed to be positive for BPV-3 DNA (using Bioreliance (BREL) qPCR Bovine Parvo Panel, Bioreliance, Rockville, MD), served as true positive samples. The positive control (PC) was confirmed by sequencing.

The acceptance criteria for this procedure required that all tested samples show a positive result using the BPV-3 assay, and that the assay did not generate a false negative result on a previously confirmed positive BPV-3 sample.

Table 11 shows the results of the BPV-3 detection in PC, which all met the acceptance criterion. Of note, the IPC v2.0 primer/probe were negative in the positive FBS and the unprocessed non-GMP bulk harvest samples containing FBS as expected, and only generated positive results when positive control plasmid (BPV-3_IPC) was used. The results of IPC qPCR reactions were as expected and met the acceptance criterion.

TABLE 11
Results of true positive samples testing.

| | | BPV-3 v3.0 | IPC v2.0 | |
|---|---|---|---|---|
| Sample | Reps | Mean ± SD CT | Mean ± SD CT | Results |
| FBS #1 | 4 | 21.78 ± 0.21 | 40.00 ± 0.00 | POS/Pass |
| FBS #2 | 4 | 25.35 ± 0.12 | 40.00 ± 0.00 | POS/Pass |
| Unprocessed non- | 6 | 29.55 ± 0.29 | 40.00 ± 0.00 | POS/Pass |
| GMP bulk harvest | | | | |
| containing FBS | | | | |
| PC | 3 | 29.21 ± 026 | 28.67 ± 0.04 | POS/Pass |
| NTC | 3 | 40.00 ± 0.00 | 40.00 ± 0.00 | NEG/Pass |

### Cross Reactivity

To test the effect of cross reactivity of the BPV-3 v3.0 and IPC v2.0 primer/probe sets, extracted nucleic acids derived from various viruses and cell lines were evaluated using the assay as described in Example 2. The closest commercially available parvoviruses to BPV-3 are Bovine parvovirus 1 (BPV-1) (ATCC® VR-767™) and Mouse minute virus (MMV, rodent parvovirus), were included to test for cross reactivity. Several other DNA viruses were included to test cross reactivity: Porcine circovirus 2 (PCV 2), Herpes simplex virus 1 (HSV 1), Pseudorabies virus (Pry). DNA from four cell lines were also tested: African green monkey kidney (VERO), baby hamster kidney cells (BHK), Chinese hamster ovary (CHO), MSV-transformed cat brain (PG 4). DNA was extracted as described in Example 2.

The accuracy of extracted nucleic acid from all viruses was confirmed using in-house specific qPCR or next generation sequencing (NGS) method.

The acceptance criterion for this procedure required that all tested species show a negative result (CT=40.00) using BPV-3 v3.0 and IPC v2.0 primer/probe sets. Table 12 shows that cross reactivity testing results revealed that all tested species were negative for BPV-3 and met the acceptance criterion.

TABLE 12
Results of cross reactivity testing.

| | | | BPV-3 v3.0 | IPC v2.0 | |
|---|---|---|---|---|---|
| Species | gene | Replicates | Mean ± SD CT | Mean ± SD CT | Results |
| Bovine parvovirus 1 | ssDNA | 6 | 40.00 ± 0.00 | 40.00 ± 0.00 | NEG/Pass |
| Mouse minute virus | ssDNA | 6 | 40.00 ± 0.00 | 40.00 ± 0.00 | NEG/Pass |
| Porcine circovirus 2 | ssDNA | 6 | 40.00 ± 0.00 | 40.00 ± 0.00 | NEG/Pass |
| Herpes simplex virus 1 | dsDNA | 6 | 40.00 ± 0.00 | 40.00 ± 0.00 | NEG/Pass |
| Pseudorabies virus | dsDNA | 6 | 40.00 ± 0.00 | 40.00 ± 0.00 | NEG/Pass |
| African green monkey | dsDNA | 6 | 40.00 ± 0.00 | 40.00 ± 0.00 | NEG/Pass |
| kidney | | | | | |
| Baby hamster kidney | dsDNA | 6 | 40.00 ± 0.00 | 40.00 ± 0.00 | NEG/Pass |
| Chinese hamster ovary | dsDNA | 6 | 40.00 ± 0.00 | 40.00 ± 0.00 | NEG/Pass |
| MSV-transformed cat | dsDNA | 6 | 40.00 ± 0.00 | 40.00 ± 0.00 | NEG/Pass |
| brain | | | | | |
| NTC | n/a | 6 | 40.00 ± 0.00 | 40.00 ± 0.00 | NEG/Pass |

### Limit of Detection (LOD)

The BPV-3 qPCR method is intended to obtain a qualitative result, proof of linearity is not required. In practice, the LOD is determined as the cut-off point in the form of the minimum number of amplified target sequences by positively ratio detected in 95% of the sample series, which is referred to as LOD95%.

To determine the sensitivity or analytical limit of detection (ALOD) of the method, BPV-3_IPC plasmid DNA was diluted at different concentrations (1, 5, 10, 20, 25, 50, 75, 100, 250, 500 GC/PCR reaction). The ALOD is a concentration of the target DNA at which an amplification product is detected with a probability of at least 0.95 (LOD95%). To test this, twelve PCR replicate measurements from each plasmid DNA concentration were evaluated in the absence of sample matrix, using the assay as described in Example 2. The concentration level with the lowest number of genome copies for which all 12 replicates were positive was considered an approximate value for LOD95%.

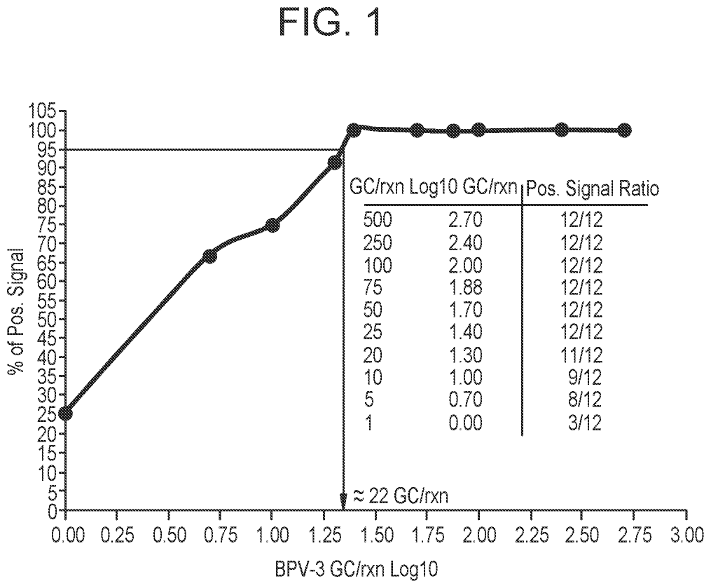

Probit analysis was employed with associated probability of 95% to calculate ALOD of BPV-3 qPCR assay. Results of analysis demonstrated that the ALOD of the BPV-3 qPCR assay is about 22 genome copies per reaction (FIG. 1).

The sample limit of detection (SLOD) is the minimum amount of target sequence that can be detected with a given level of confidence (LOD95%) in presence of sample matrix. Experiments to determine the SLOD were performed in the same manner as the ALOD experiments; with the exception that positive control plasmid DNA at different level of concentrations (25, 50, 75 and 100 GC/reaction) were spiked into the extracted DNA from the different sample matrices rather than molecular biology grade water (MBGW).

In this study, different sample matrices were used, see Table 13. Each sample was spiked with 25, 50, 75 and 100 genome copies (BPV-3_IPC positive control plasmid) per reaction using a QIAgility liquid handler (QIAGEN, Inc.) and tested using the BPV-3 qPCR detection assay described in Example 2. All samples were evaluated in 12 replicates. Table 14 and 15 present the results of the SLOD. Results revealed that the SLOD of the BPV-3 qPCR assay was 25 genome copies per PCR reaction. Similar results were achieved for the IPC qPCR assay (Table 16).

TABLE 13
Matrix samples used for SLOD determination.

| Sample | DNA amount |
|---|---|
| FBS | 10 μL DNA/qPCR reaction |
| Vero | 30 ng/10 μL DNA/qPCR reaction |
| BHK | 30 ng/10 μL DNA/qPCR reaction |
| CHO | 50 ng/10 μL DNA/qPCR reaction |

TABLE 14
BPV-3 assay SLOD results using FBS and Vero cells.

| | Sample matrix | | | | | | | |
|---|---|---|---|---|---|---|---|---|
| | FBS spiked with PC | | | | Vero spiked with PC | | | |
| Replicates and GC/rxn | 25 | 50 | 75 | 100 | 25 | 50 | 75 | 100 |
| rep 1 | 34.35 | 34.15 | 32.41 | 32.65 | 34.83 | 33.82 | 33.54 | 31.94 |
| rep 2 | 34.90 | 34.68 | 32.77 | 32.47 | 34.71 | 34.45 | 33.23 | 32.97 |
| rep 3 | 34.35 | 33.80 | 32.52 | 32.35 | 34.91 | 33.70 | 33.45 | 33.08 |
| rep 4 | 34.32 | 34.31 | 33.04 | 32.71 | 34.75 | 34.01 | 33.73 | 33.11 |
| rep 5 | 32.96 | 33.87 | 32.67 | 32.57 | 35.38 | 34.22 | 32.85 | 33.00 |
| rep 6 | 35.13 | 33.39 | 33.20 | 32.97 | 34.04 | 33.86 | 32.97 | 33.16 |
| rep 7 | 34.63 | 34.08 | 33.24 | 33.05 | 35.96 | 34.32 | 33.43 | 32.97 |
| rep 8 | 35.78 | 34.57 | 33.21 | 33.39 | 34.44 | 33.71 | 33.40 | 33.10 |
| rep 9 | 33.06 | 34.33 | 32.80 | 33.30 | 36.55 | 33.43 | 33.46 | 33.30 |
| rep 10 | 35.04 | 33.61 | 33.39 | 32.96 | 34.78 | 34.12 | 33.54 | 32.96 |
| rep 11 | 34.78 | 33.70 | 33.41 | 32.97 | 34.29 | 33.69 | 33.18 | 32.95 |
| rep 12 | 34.45 | 34.56 | 33.38 | 33.17 | 36.01 | 34.01 | 33.31 | 33.62 |
| Mean | 34.48 | 34.09 | 33.00 | 32.88 | 35.05 | 33.95 | 33.34 | 33.01 |
| Positive signal ratio | 12/12 | 12/12 | 12/12 | 12/12 | 12/12 | 12/12 | 12/12 | 12/12 |
| Positive signal ratio % | 100 | 100 | 100 | 100 | 100 | 100 | 100 | 100 |

TABLE 15
BPV-3 assay SLOD results using BHK and CHO cells.

| | Sample matrix | | | | | | | |
|---|---|---|---|---|---|---|---|---|
| | BHK spiked with PC | | | | CHO spiked with PC | | | |
| Replicates and GC/rxn | 25 | 50 | 75 | 100 | 25 | 50 | 75 | 100 |
| rep 1 | 34.84 | 34.23 | 33.05 | 32.39 | 34.55 | 34.07 | 32.58 | 33.31 |
| rep 2 | 34.22 | 33.94 | 33.20 | 32.14 | 34.17 | 33.46 | 32.16 | 32.74 |
| rep 3 | 35.22 | 33.70 | 32.97 | 32.96 | 34.37 | 33.61 | 33.06 | 32.77 |
| rep 4 | 34.50 | 33.96 | 33.26 | 32.96 | 33.44 | 33.60 | 32.54 | 31.99 |
| rep 5 | 34.09 | 34.29 | 33.12 | 32.96 | 34.24 | 32.51 | 32.94 | 33.35 |
| rep 6 | 34.29 | 33.54 | 32.99 | 32.55 | 33.79 | 33.43 | 33.11 | 33.47 |
| rep 7 | 34.62 | 34.19 | 33.43 | 32.81 | 34.18 | 33.36 | 33.58 | 33.34 |
| rep 8 | 34.18 | 33.67 | 33.31 | 32.95 | 34.47 | 33.50 | 32.68 | 33.15 |
| rep 9 | 33.96 | 34.10 | 33.11 | 32.16 | 33.93 | 33.53 | 33.68 | 32.10 |
| rep 10 | 34.78 | 33.44 | 32.78 | 32.52 | 34.44 | 33.51 | 33.36 | 32.60 |
| rep 11 | 34.20 | 33.91 | 33.12 | 32.63 | 34.25 | 34.09 | 33.05 | 32.85 |
| rep 12 | 34.25 | 33.45 | 32.87 | 32.99 | 35.01 | 33.51 | 32.29 | 32.94 |
| Mean | 34.43 | 33.87 | 33.10 | 32.67 | 34.24 | 33.51 | 32.92 | 32.88 |
| Positive signal ratio | 12/12 | 12/12 | 12/12 | 12/12 | 12/12 | 12/12 | 12/12 | 12/12 |
| % of positive signal | 100 | 100 | 100 | 100 | 100 | 100 | 100 | 100 |

TABLE 16
IPC assay SLOD results using FBS and Vero cells.

| | Sample matrix | | | | | | | |
|---|---|---|---|---|---|---|---|---|
| | FBS spiked with PC | | | | Vero spiked with PC | | | |
| Replicates and GC/rxn | 25 | 50 | 75 | 100 | 25 | 50 | 75 | 100 |
| rep 1 | 34.22 | 33.23 | 32.28 | 32.32 | 35.02 | 33.97 | 32.13 | 32.09 |
| rep 2 | 33.69 | 32.76 | 31.83 | 31.86 | 33.88 | 34.19 | 31.95 | 32.19 |
| rep 3 | 34.08 | 34.20 | 32.19 | 31.48 | 34.61 | 33.82 | 32.17 | 30.96 |
| rep 4 | 34.54 | 33.74 | 32.49 | 32.15 | 33.54 | 33.77 | 32.39 | 32.17 |
| rep 5 | 33.75 | 33.70 | 32.17 | 32.21 | 33.81 | 33.79 | 32.01 | 31.77 |
| rep 6 | 33.88 | 33.78 | 32.19 | 32.37 | 34.46 | 33.45 | 32.08 | 32.47 |
| rep 7 | 35.36 | 33.88 | 32.36 | 31.94 | 34.37 | 33.26 | 31.98 | 31.98 |
| rep 8 | 34.34 | 33.83 | 32.06 | 32.34 | 34.25 | 34.24 | 32.08 | 31.69 |
| rep 9 | 31.13 | 33.54 | 32.22 | 32.26 | 34.26 | 33.81 | 31.80 | 31.99 |
| rep 10 | 33.79 | 33.45 | 30.79 | 29.74 | 34.51 | 33.51 | 32.45 | 31.30 |
| rep 11 | 35.83 | 33.90 | 31.97 | 31.98 | 35.57 | 33.70 | 32.35 | 32.00 |
| rep 12 | 35.17 | 33.31 | 32.57 | 31.38 | 34.64 | 33.25 | 32.11 | 31.55 |
| Mean | 34.15 | 33.61 | 32.09 | 31.84 | 34.41 | 33.73 | 32.12 | 31.85 |
| Positive signal ratio | 12/12 | 12/12 | 12/12 | 12/12 | 12/12 | 12/12 | 12/12 | 12/12 |
| % of positive signal | 100 | 100 | 100 | 100 | 100 | 100 | 100 | 100 |

### Robustness

To evaluate the robustness of the BPV-3 qPCR detection assay, unprocessed non-GMP bulk harvest samples containing FBS, and FBS samples were tested (Table 17). The acceptance criterion would be to meet the proposed system suitability and assay acceptance criteria and sample acceptance criteria as described in Example 2. The samples were previously confirmed to be negative or positive for BPV-3 DNA by alternative methods such as NGS, sanger DNA sequencing or Bioreliance (BREL) Bovine Parvo Panel qPCR assay.

Samples were subjected to DNA extraction as described in Example 2. All controls were prepared, and qPCR executed in parallel with the test sample and then subjected to the BPV-3 qPCR detection assay as described in Example 2. The results of BPV-3 qPCR assay were interpreted as described in Example 2 and reported as “positive” or “negative”.

TABLE 17
Test samples used for BPV-3 assay robustness evaluation.

| Sample | BPV-3 status |
|---|---|
| FBS | negative |
| irradiated FBS #1 | negative |
| characterized FBS | negative |
| unprocessed non-GMP bulk harvest sample | positive |
| containing FBS # 1 | |
| unprocessed non-GMP bulk harvest sample | positive |
| containing FBS # 2 | |
| irradiated FBS #2 | positive |
| irradiated FBS #3 | positive |

The results of negative test samples from Table 17 are shown in Tables 18-20. The validity of the assay was demonstrated with results of assay controls including negative results in NTC and NEC for BPV-3 and IPC assays in 3 out of 3 PCR reaction replicates; detection of target amplification signal in PC with BPV-3 and IPC assays in 3 out of 3 PCR reaction replicates; detection of amplification signal with BPV-3 and IPC assays for PEC in 3 out of 3 PCR reaction replicates indicating an accurate extraction recovery; detection of BPV-3 in spiked test sample (+S) in 3 out of 3 PCR reaction replicates and ΔCT results indicating no sample matrix interference. Test samples were negative with the IPC assay in 3 out of 3 PCR reaction replicates indicating the absence of cross contamination between positive control and sample, also the test samples were negative with BPV-3 assay in 3 out of 3 PCR reaction replicates indicating absence of BPV-3 DNA. Altogether, BPV-3 assay results indicate that assay and sample acceptance criteria were met and passed, and test samples were valid and “negative” for BPV-3.

TABLE 18
Results for FBS Sample

| qPCR reaction controls/samples | BPV-3 assay CT | | | IPC assay CT | | | Results BPV-3/IPC |
|---|---|---|---|---|---|---|---|
| No template control (NTC) | 40.00 | 40.00 | 40.00 | 40.00 | 40.00 | 40.00 | NEG/NEG |
| Negative extraction control (NEC) | 40.00 | 40.00 | 40.00 | 40.00 | 40.00 | 40.00 | NEG/NEG |
| Positive extraction control (PEC) | 28.84 | 28.89 | 28.72 | 28.04 | 28.22 | 28.33 | POS/POS |
| Positive control (PC) | 28.18 | 29.12 | 29.33 | 28.34 | 28.43 | 28.78 | POS/POS |
| Inhibition control (IHN) | 29.96 | 29.97 | 30.16 | n/a | | | POS |
| Sample matrix interference (ΔCT) | 1.15 | | | n/a | | | met |
| Test sample | 40.00 | 40.00 | 40.00 | 40.00 | 40.00 | 40.00 | NEG/NEG |

| | |
|---|---|
| System suitability and assay acceptance criteria | Passed |
| Sample results acceptance criteria | Passed |
| Test sample result | Valid and Negative |

TABLE 19
Results for irradiated FBS #1

| qPCR reaction controls/samples | BPV-3 assay CT | | | IPC assay CT | | | Results BPV-3/IPC |
|---|---|---|---|---|---|---|---|
| No template control (NTC) | 40.00 | 40.00 | 40.00 | 40.00 | 40.00 | 40.00 | NEG/NEG |
| Negative extraction control (NEC) | 40.00 | 40.00 | 40.00 | 40.00 | 40.00 | 40.00 | NEG/NEG |
| Positive extraction control (PEC) | 28.00 | 28.06 | 27.87 | 27.16 | 27.16 | 27.41 | POS/POS |
| positive control (PC) | 27.70 | 28.72 | 28.78 | 28.10 | 28.39 | 28.55 | POS/POS |
| Inhibition control (IHN) | 28.42 | 28.87 | 29.00 | n/a | | | POS |
| Sample matrix interference (ΔCT) | 0.36 | | | n/a | | | met |
| Test sample | 40.00 | 40.00 | 40.00 | 40.00 | 40.00 | 40.00 | NEG/NEG |

| | |
|---|---|
| System suitability and assay acceptance criteria | Passed |
| Sample results acceptance criteria | Passed |
| Test sample result | Valid and Negative |

TABLE 20
Results for characterized FBS

| qPCR reaction controls/samples | BPV-3 assay CT | | | IPC assay CT | | | Results BPV-3/IPC |
|---|---|---|---|---|---|---|---|
| No template control (NTC) | 40.00 | 40.00 | 40.00 | 40.00 | 40.00 | 40.00 | NEG/NEG |
| Negative extraction control (NEC) | 40.00 | 40.00 | 40.00 | 40.00 | 40.00 | 40.00 | NEG/NEG |
| Positive extraction control (PEC) | 28.48 | 28.72 | 28.57 | 28.01 | 28.33 | 27.95 | POS/POS |
| positive control (PC) | 28.95 | 29.46 | 29.24 | 28.67 | 28.71 | 28.63 | POS/POS |
| Inhibition control (IHN) | 29.03 | 28.74 | 29.29 | n/a | | | POS |
| Sample matrix interference (ΔCT) | 0.19 | | | n/a | | | met |
| Test sample | 40.00 | 40.00 | 40.00 | 40.00 | 40.00 | 40.00 | NEG/NEG |

| | |
|---|---|
| System suitability and assay acceptance criteria | Passed |
| Sample results acceptance criteria | Passed |
| Test sample result | Valid and Negative |

The BPV-3 assay results for the positive test samples (two unprocessed non-GMP bulk harvest samples containing FBS and two FBS samples (FBS #2 and FBS #3) are shown in Tables 21-24. The validity of the assay was demonstrated, the results of the assay controls including the expected with negative results for NTC and NEC for BPV-3 and IPC assays in 3 out of 3 PCR reaction replicates; and detection of target amplification signal in PC with BPV-3 assay in 3 out of 3 PCR reaction replicates.

When a test sample showed a positive amplification signal and detection of BPV-3, evaluation of the spiked test sample (IHC) and ΔCT became irrelevant and did not need to be considered as a part of assay acceptance criteria. Detection of amplification signal with BPV-3 assay and IPC assays for PEC in 3 out of 3 PCR reaction replicates indicated an accurate extraction recovery; test sample negative signal with IPC assay in 3 out of 3 PCR reaction replicates indicated the absence of cross contamination between positive control and sample. Test sample presented a positive amplification signal with BPV-3 assay in 3 out of 3 PCR reaction replicates indicating the presence of BPV-3 DNA. Altogether, results indicated that assay and sample acceptance criteria were met and passed, and the test sample was valid and positive for BPV-3.

TABLE 21
Results for unprocessed non-GMP bulk harvest sample 1

| qPCR reaction controls/samples | BPV-3 assay CT | | | IPC assay CT | | | Results BPV-3/IPC |
|---|---|---|---|---|---|---|---|
| No template control (NTC) | 40.00 | 40.00 | 40.00 | 40.00 | 40.00 | 40.00 | NEG/NEG |
| Negative extraction control (NEC) | 40.00 | 40.00 | 40.00 | 40.00 | 40.00 | 40.00 | NEG/NEG |
| Positive extraction control (PEC) | 28.39 | 28.46 | 28.14 | 27.81 | 27.91 | 27.68 | POS/POS |
| positive control (PC) | 28.49 | 28.42 | 28.35 | 28.15 | 28.02 | 28.06 | POS/POS |
| Inhibition control (IHN) | n/a | | | n/a | | | n/a |
| Sample matrix interference (ΔCT) | n/a | | | n/a | | | n/a |
| Test sample | 29.36 | 29.21 | 29.52 | 40.00 | 40.00 | 40.00 | POS/NEG |

| | |
|---|---|
| System suitability and assay acceptance criteria | Passed |
| Sample results acceptance criteria | Passed |
| Test sample result | Valid and Positive |

TABLE 22
Results for unprocessed non-GMP bulk harvest sample 2

| qPCR reaction controls/samples | BPV-3 assay CT | | | IPC assay CT | | | Results BPV-3/IPC |
|---|---|---|---|---|---|---|---|
| No template control (NTC) | 40.00 | 40.00 | 40.00 | 40.00 | 40.00 | 40.00 | NEG/NEG |
| Negative extraction control (NEC) | 40.00 | 40.00 | 40.00 | 40.00 | 40.00 | 40.00 | NEG/NEG |
| Positive extraction control (PEC) | 28.61 | 28.57 | 28.42 | 27.99 | 28.13 | 27.81 | POS/POS |
| positive control (PC) | 28.53 | 28.38 | 28.37 | 27.98 | 27.92 | 27.94 | POS/POS |
| Inhibition control (IHN) | n/a | | | n/a | | | n/a |
| Sample matrix interference (ΔCT) | n/a | | | n/a | | | n/a |
| Test sample | 28.76 | 28.79 | 28.88 | 40.00 | 40.00 | 40.00 | POS/NEG |

| | |
|---|---|
| System suitability and assay acceptance criteria | Passed |
| Sample results acceptance criteria | Passed |
| Test sample result | Valid and Positive |

TABLE 23
Results for FBS #2

| qPCR reaction controls/samples | BPV-3 assay CT | | | IPC assay CT | | | Results BPV-3/IPC |
|---|---|---|---|---|---|---|---|
| No template control (NTC) | 40.00 | 40.00 | 40.00 | 40.00 | 40.00 | 40.00 | NEG/NEG |
| Negative extraction control (NEC) | 40.00 | 40.00 | 40.00 | 40.00 | 40.00 | 40.00 | NEG/NEG |
| Positive extraction control (PEC) | 28.39 | 28.43 | 28.52 | 27.87 | 27.93 | 28.12 | POS/POS |
| positive control (PC) | 28.21 | 28.25 | 28.44 | 28.13 | 27.91 | 27.95 | POS/POS |
| Inhibition control (IHN) | n/a | | | n/a | | | n/a |
| Sample matrix interference (ΔCT) | n/a | | | n/a | | | n/a |
| Test sample | 21.31 | 21.43 | 21.53 | 40.00 | 40.00 | 40.00 | POS/NEG |

| | |
|---|---|
| System suitability and assay acceptance criteria | Passed |
| Sample results acceptance criteria | Passed |
| Test sample result | Valid and Positive |

TABLE 24
Results for FBS #3

| qPCR reaction controls/samples | BPV-3 assay CT | | | IPC assay CT | | | Results BPV-3/IPC |
|---|---|---|---|---|---|---|---|
| No template control (NTC) | 40.00 | 40.00 | 40.00 | 40.00 | 40.00 | 40.00 | NEG/NEG |
| Negative extraction control (NEC) | 40.00 | 40.00 | 40.00 | 40.00 | 40.00 | 40.00 | NEG/NEG |
| Positive extraction control (PEC) | 28.47 | 28.58 | 28.61 | 28.08 | 28.10 | 28.29 | POS/POS |
| positive control (PC) | 28.45 | 28.44 | 28.40 | 27.83 | 27.89 | 27.69 | POS/POS |
| Inhibition control (IHN) | n/a | | | n/a | | | n/a |
| Sample matrix interference (ΔCT) | n/a | | | n/a | | | n/a |
| Test sample | 24.98 | 25.09 | 25.22 | 40.00 | 40.00 | 40.00 | POS/NEG |

| | |
|---|---|
| System suitability and assay acceptance criteria | Passed |
| Sample results acceptance criteria | Passed |
| Test sample result | Valid and Positive |

### Repeatability

To test the repeatability, the extracted DNA from BHK and Vero cells (30 ng/PCR reaction) were prepared either as unspiked or spiked with 25 genome copies of BPV-3_IPC positive control plasmid (spiked at the SLOD level). The BPV-3 detection assay (as described in Example 2) was performed in 12 PCR reaction replicates on different days.

The acceptance criterion for this procedure required that at least 11 out of the 12 replicates show no amplification signal for unspiked samples by BPV-3 and IPC qPCR assays and at least 11 out of 12 samples showed a positive amplification signal for spiked samples by BPV-3 and IPC qPCR assays. The results of the repeatability assay revealed that 12 out of 12 unspiked (Table 25) and spiked (Table 26) met the acceptance criteria demonstrating the repeatability of the BPV-3 detection assay.

TABLE 25
Repeatability of BPV-3 qPCR on unspiked test samples.

| | PCR | | | | | BPV-3 and |
|---|---|---|---|---|---|---|
| | Reaction | BPV-3 | IPC | Negative | Negative | IPC qPCR |
| Sample | Replicates | assay CT | assay CT | signal ratio | signal % | assay results |
| Vero | 12 | 40.00 | 40.00 | 12 out of 12 | 100 | NEG/Passed |
| BHK | 12 | 40.00 | 40.00 | 12 out of 12 | 100 | NEG/Passed |

TABLE 26
Intra- and inter-assay repeatability of BPV-3 qPCR
results on spiked test sample at two different days.
Spiked (25 GC/PCR reaction)

| | Assay | | | | | |
|---|---|---|---|---|---|---|
| | BPV-3 CT | | | | IPC CT | |
| | Sample matrix | | | | | |
| PCR Reaction | BHK | | Vero | | BHK | Vero |
| replicate & repeat | Day 1 | Day 2 | Day 1 | Day 2 | One run | |
| rep 1 | 34.30 | 34.84 | 34.63 | 34.83 | 33.76 | 34.44 |
| rep 2 | 34.59 | 34.22 | 35.17 | 34.71 | 34.17 | 34.95 |
| rep 3 | 34.47 | 35.22 | 35.14 | 34.91 | 34.65 | 34.18 |
| rep 4 | 34.97 | 34.50 | 34.68 | 34.75 | 32.81 | 34.32 |
| rep 5 | 34.21 | 34.09 | 34.86 | 35.38 | 34.24 | 34.30 |
| rep 6 | 33.23 | 34.29 | 35.95 | 34.04 | 33.71 | 33.96 |
| rep 7 | 34.85 | 34.62 | 35.69 | 35.96 | 32.91 | 34.44 |
| rep 8 | 33.66 | 34.18 | 35.03 | 34.44 | 33.99 | 34.60 |
| rep 9 | 34.28 | 33.96 | 34.72 | 36.55 | 33.92 | 34.05 |
| rep 10 | 34.41 | 34.78 | 34.17 | 34.78 | 34.17 | 34.19 |
| rep 11 | 34.47 | 34.20 | 34.14 | 34.29 | 33.83 | 34.32 |
| rep 12 | 34.94 | 34.25 | 34.46 | 36.01 | 33.68 | 34.16 |
| Positive signal ratio | 12/12 | 12/12 | 12/12 | 12/12 | 12/12 | 12/12 |
| % of positive signal | 100 | 100 | 100 | 100 | 100 | 100 |
| Results | POS/ | POS/ | POS/ | POS/ | POS/ | POS/ |
| | Passed | Passed | Passed | Passed | Passed | Passed |
| Mean CT | 34.37 | 34.43 | 34.89 | 35.05 | 33.82 | 34.33 |
| StdDev CT | 0.51 | 0.37 | 0.55 | 0.76 | 0.53 | 0.26 |
| CV % | 1.48 | 1.07 | 1.58 | 2.18 | 1.55 | 0.77 |

In summary, all tested samples met required qualification specifications for a qualitative assay including specificity, limit of detection, robustness, and repeatability. The qualified BPV-3 detection assay allows for robust detection of 25 BPV-3 genome copies per PCR reaction. All tested samples met the proposed system suitability and assay acceptance criteria as well as test sample acceptance criteria. Thus, the qualified BPV-3 real-time PCR assay allowed the detection of BPV-3 DNA in test sample.

### Example 4 Bovine Parvovirus 3 (BPV-3) Real-Time Quantitative Polymerase Chain Reaction Assay

### Sample Preparation

For test samples that contained cells, the cells were subjected to a low speed centrifugation (LSC) before DNA extraction, 320×g, for 10 minutes at room temperature. The supernatant was collected for DNA extraction. Cell free samples were processed directly.

To 250 μL of a test sample was added 10 μL of an RNase Cocktail™ Enzyme Mix (Thermo Fisher, Carlsbad, CA) and 0.5M EDTA, pH 8.0 (9 μL:1 μL) mixture. An AutoMate Express™ Nucleic Acid Extraction System (ThermoFisher) was used to extract 200 μL of the test sample. On the AutoMate, the PrepSEQ (PS) Express 1-2-3 protocol was selected for the DNA extraction procedure with the following parameters: 1-hour proteinase K (PK) digestion and 100 μL elution. Extracted DNA was visually inspected and if there were any residual magnetic beads in the eluted DNA, a DynaMag™-2 Magnet benchtop workstation (ThermoFisher) was used the remove any remaining beads in the eluted DNA. The DNA was used immediately or stored at −20 C.

Alongside the test samples, a BPV-3_IPC (PEC) plasmid control and a negative extraction control (NEC) were also prepared and run as described above. For the positive extraction control, 250 μL sterile 1× phosphate buffered saline (PBS) was spiked with 62,000 genome copies (GC) BPV-3_IPC plasmid DNA. This was used to evaluate DNA extraction performance during test sample preparation and verified that assay does not produce false negative results. Assuming 100% efficiency of DNA extraction, final eluted DNA should contain 500 GC/μL. For the negative extraction control, ˜300 ng human genomic DNA in 1×PBS was used. The NEC was used to monitor cross contamination during nucleic acid extraction and verifies that assay did not produce false positive results with non-specific background DNA.

### Preparation of Other Assay Controls

No template control (NTC): Contained all the PCR reagents except for the DNA template. This was used as a negative control to verify qPCR raw materials were free of any DNA contamination.

Positive control (PC): Three PC concentrations were included, 2E4, 2E5, and 2E7 GC/reaction of BPV-3_IPC positive control plasmid DNA. This was used to verify that qPCR components work accurately and should have PCR amplification signal specific to target sequence.

Positive extraction control (PEC): The ˜400 ng human genomic DNA (˜3 μL) added to 250 μL if 1× PBS and is also spiked with 62,500 copies of BPV-3_IPC positive control plasmid DNA (6.25 μL) followed by extraction.

Inhibition control (IHN): Contains extracted DNA from the test sample which is spiked with BPV-3_IPC plasmid DNA at 2E4 GC/reaction. This is used to determine sample interference/inhibition level.

Standard (ST): A 10-fold dilution of BPV-3_IPC plasmid DNA (1E8, 1E7, 1E6, 1E5, 1E4, 1E3 GC/reaction) used to determine PCR efficiency, linear range, and to quantify the absolute copy number of target sequence in the test sample/control.

BPV-3 qPCR and IPC qPCR Detection Assay Setups

The BPV-3 qPCR detection assay setup contained the BPV-3 v3.0 prime/probe set to detect BPV-3 DNA in test sample and the IPC v2.0 primer/probe set to monitor any accidental cross contamination between positive samples and negative samples. Two different master mixes were prepared, a BPV-3 qPCR master mix and an IPC qPCR master mix.

The BPV-3 v3.0 prime/probe master mix comprised the BPV-3 v3.0 Primer-Probe, water, and 2× TAQMAN® Universal PCR Master Mix (Applied Biosystems). The TAQMAN® master mix contained the essential components for the qPCR reaction including optimized buffers to amplify G/C-rich sequence, dNTPs with dUTP, AmpliTaq Gold™ DNA polymerase, ROX dye (as a passive internal reference) and AmpErase™ UNG (Uracil-DNA Glycosylase enzyme to eliminate carry-over contamination PCR products containing dU). Table 27 provides the amount of each reaction component for each sample tested, the test sample and the five controls: negative extraction control (NEC), no template control (NTC), positive extraction control (PEC), inhibition control (IHN), and positive control (PC).

TABLE 27
Reaction components for BPV_3 v3.0 quantitative assay

| | Stock | Reaction | |
|---|---|---|---|
| Reagent | concentration | concentration | |
| BPV-3 v3.0 primer/probe set, Assay | 60X | 1X | |
| ID: AP47Y2C | | | |
| IPC v2.0 primer/probe set, Assay | 60X | 1X | |
| ID: APGZJVP | | | |
| ABI Universal TaqMan PCR master | 2X | 1X | |
| mix 2X | | | |
| Extracted DNA from test sample | n/a | 10 | μL |
| Human genomic DNA | Variable | ~300 | ng |

| | | |
|---|---|---|
| Standard/Controls | n/a | variable |
| Water | n/a | up to 30 μL |

Each reaction for samples and controls was performed in triplicate (3 wells) in a 96 well reaction plate. The qPCR reaction set up could be performed manually or using a liquid handler robot, such as QIAgility HEPA/UV liquid handler (Qiagen, Inc., Germantown, MD). A MicroAmp Optical 96-well plate (Applied Biosystems) was used to accommodate the qPCR reaction mixtures and then covered with MicroAmp Optical Adhesive Film (Applied Biosystems). The amount of PCR reagent components, sample, and controls in each PCR reaction is listed in Table 27 and 28. The sample layout on 96-well plate can be set up per analyst, and the sealed plate loaded into the a QuantStudior™ 7 Flex Real-Time PCR System (Applied Bioscience).

A QuantStudio™ 7 Flex Real-Time PCR System (Applied Bioscience) was used to perform the qPCR reactions. The thermal cycling program was set for the following reaction times and temperatures: 50° C. 2 min (AmpEraser™ UNG activation), 95° C. 10 min (AmpliTaq Gold™ DNA polymerase activation/denaturation), and 40 cycle of 95° C. 15 sec, 60° C. 1 min (annealing/extension/data collection). Table 28 provides the experimental properties that were selected.

TABLE 28
QuantStudio ™ software experimental
properties for BPV-3 and IPC qPCR.

| Experimental property name: | Experimental property selected: |
|---|---|
| Instrument | QuantStudio ™ 7 Flex System |
| Block | 96-Well (0.2 mL) |
| Experiment | Standard Curve |
| Reagent | TAQMAN ® Reagents |
| Run Properties | Standard |
| Reporter | FAM (for BPV-3 v3.0 primer/probe set) |
| | VIC (for IPC v2.0 primer/probe set) |
| Quencher | MGB-NFQ |
| Passive Dye | ROX |
| Threshold | ΔRn 0.1 for BPV-3 assay |
| | ΔRn 0.2 for IPC assay |
| Baseline | Automatic |
| Reaction volume | 30 μL |

The assay utilizes the QS7 real-time PCR system (ThermoFisher Scientific, Waltham, MA) in combination with BPV_3 and IPC primers and probes described herein.

The assay must meet the following assay acceptance criteria to be considered a valid assay, see Table 29. If any of the assay acceptance criteria are not met, the assay should be repeated.

TABLE 29
Valid assay acceptance criteria

| | |
|---|---|
| Control | Valid assay acceptance criteria |

BPV 3

| | |
|---|---|
| No | A minimum of 2 out of 3 reactions must show no amplification signal, CT = |
| Template | 40.00 (undetermined) and the obtained mean of GC quantity per reaction |
| Control | (if any) shall be less than the assay LOD95% (27 GC/reaction). |
| Negative | Any CT value reported as “Undetermined” represents the absence of DNA |
| Extraction | and can be converted to 40.00 for statistical analysis. |
| Control | |
| Positive | A minimum of 2 out of 3 reactions must show an amplification signal of CT < |
| Extraction | 40.00, and the obtained mean GC of quantity per reaction greater than |
| Control | assay LOQ (1E3 GC/rxn). |
| Positive | A minimum of 2 out of 3 reactions must show amplification signal of CT < |
| control | 40.00, and the obtained mean GC of quantity per reaction must meet the |
| PC_2E4 | established assay accuracy criteria, ±30% of PC reference value: |
| PC_2E5 | PC_2E7 acceptance accuracy range: 1.4E7 to 2.6E8 GC/rxn |
| PC_2E7 | PC_2E5 acceptance accuracy range: 1.4E5 to 2.6E6 GC/rxn |
| | PC_2E4 acceptance accuracy range: 1.4E4 to 2.6E4 GC/rxn |
| Inhibition | A minimum of 2 out of 3 reactions must show amplification signal of CT < |
| Control | 40.00, and the obtained mean GC of quantity per reaction shall be within |
| (IHN or +S) | one magnitude of the original (2E4) spiked concentration level; the INH |
| | mean GC of quantity should be within 2E3 to 2E5 GC/reaction. |
| | INH control is irrelevant and not applicable if test sample is positive with |
| | BPV-3 with mean GC of quantity of equal or greater than 2E4 per reaction. |
| Standard | Correlation coefficient (R²) ≥ 0.98 |
| | PCR amplification efficiency within 90 to 110%. |

IPC

| | |
|---|---|
| Negative | A minimum of 2 out of 3 reactions must show no amplification signal, CT = |
| Extraction | 40.00 (undetermined). |
| Control | |
| Positive | A minimum of 2 out of 3 reactions must show amplification signal of CT < |
| Extraction | 40.00, when the IPC is the target sequence. |
| Control | |
| Test Sample | A minimum of 2 out of 3 reactions must show no amplification signal, CT = |
| | 40.00 (undetermined), when the IPC is the target sequence. |

Statistical analysis of the data was carried out with Excel software (Microsoft, Redmond, WA) and/or TIBCO SPOTFIRE© software (Palo Alto, CA). Any CT value reported as “Undetermined” represented the absence of target DNA and was converted to 40.00 for further statistical analysis. The sample, standard, and controls were tested in 3 replicates. The outlier rule based on Grubbs' test or Dixon's Q test may be applied to the CT values from samples, controls or standards. An outlier analysis test may be applied to the triplicates data points to determine if one of the results could be omitted from the analysis as an outlier. If outlier exclusion is statistically significant (significance level: α=0.05) and is allowed, repeat data analysis with valid duplicate results. Results based on duplicate data points must meet all acceptance criteria for the controls and standard to be valid. Determination of outlier (two-sided test with significant level of alpha 0.05; may be tested using online or offline resources known to those of skill in the art. Examples of reporting and interpretation of data is provided in Table 30.

TABLE 30
Reporting and interpretation of data.

| Test sample (BPV-3 GC/rxn) | Report | Interpretation |
|---|---|---|
| BPV-3 CT value was | BPV-3 Not Detected | Target sequence not detected or |
| Undetermined (CT = 40.00) | | detected at a level below the |
| indicating the absence of BPV-3 | | LOD95% of the assay. |
| or the mean of the BPV-3 GC/rxn | | |
| wss equal or less than assay | | |
| LOD95% (27 GC/rxn) | | |
| Mean GC/rxn of BPV-3 was | BPV-3 Detected | Target sequence was detected, but |
| less than assay LOQ (1E3) and | (quantity as Log10 of mean ± | at a level below the LOQ of the |
| greater than assay LOD95% | StdDev per mL provided) | assay; not accurately quantifiable. |
| (27 GC/rxn) | | |
| Mean of BPV-3 GC/rxn was | BPV-3 Detected | Target sequence was detected, and |
| within the dynamic range of the | (quantity as Log10 of mean ± | quantification was accurate and |
| assay (1E3 to 1E8 GC/rxn) | StdDev per mL provided) | within the linear range of the assay. |
| Mean GC/rxn of BPV-3 was | BPV-3 Detected | Target sequence was detected and |
| greater than upper set point | (quantity as Log10 of mean ± | was greater than the upper |
| (1E8) of assay dynamic range | StdDev per mL provided) | dynamic range of the assay. |
| | Alternatively, the quantity may | The assay can be repeated with |
| | be reported as greater than the | different dilution factors to allow |
| | upper level of the dynamic | for quantification within |
| | range of the assay. | operational range. |

### Example 5 Quantitation Assay Qualification

### Test Sample Results Acceptance Criteria

Qualification of the quantitative assay was performed. The following parameters were qualified: specificity, limit of detection (LOD), linearity, dynamic range, precision including repeatability (intra-assay specificity) and intermediate precision (within-laboratory precision), accuracy, limit of quantification (LOQ), and robustness and assay verification was determined.

### Specificity: Sample Matrix Effect (to Test the Absence of False Positives)

To demonstrate that the assay did not generate false positive results (an error in which qPCR incorrectly results in a positive in the absence of the target sequence) the following sample matrixes were tested.

Cell lines tested: PG4 (Cat brain Moloney sarcoma virus-transformed), Vero (African green monkey kidney), 324K (SV40-transformed human newborn kidney), L929 (Mouse fibroblast cell line), CHO (Chinese hamster ovary), HEK293 (human embryonic kidney), MDBK (Madin-Darby bovine kidney), NRK (Normal Rat kidney), human white blood cells (Promega, San Luis Obispo, CA). Media tested: DMEM, McCoy 9A, HAM F12. Animal serum tested: fetal bovine serum and horse serum.

Acceptance criterion required that all tested samples show a negative result in at least 5 out of 6 PCR replicates. No false positives were detected in 6 out of 6 replicates.

### Specificity: BPV-3 Detection (to Test the Absence of False Negatives)

To demonstrate that the assay did not generate false negative results (an error in which qPCR incorrectly shows negative in the presence of the target sequence) the above-mentioned cell lines, media, and animal serums were spiked with a BPV-3 positive control plasmid (50,000 genome copies/200 μL of sample) followed by DNA extraction and BPV-3 qPCR assay as described in Example 4. The acceptance criteria required that at least 5 out of 6 replicates show a positive result. All of the tested samples showed 6 out of 6 replicates detecting BVP-3. No false negatives were detected.

### Specificity: Cross Reactivity

To determine the cross reactivity of the assay, closely related viral species/isolates were evaluated for exclusivity and inclusivity.

Exclusivity demonstrated that the assay did not detect non-target viral species/isolates which are closely related to the target sequence. BPV-1 (Bovine Parvovirus 1, VR-767™ ATCC®, Manassas, VA), BPV-2 (Bovine Parvovirus 2) and MMVp (Mouse Minute Virus prototype) viruses were extracted as described above. Since BPV-2 is not commercially available, full length non-structural (NS) gene sequence of BPV-2 (GenBank accession number: NC_006259) was synthesized and cloned in a plasmid and verified by sequencing. The plasmid was used as a BPV-2 reference isolate with concentration of 1E6 GC per PCR reaction.

The viral species/isolates were used in the BPV-3 qPCR assay as described in Example 4. The acceptance criterion required that all tested samples show a negative result in at least 5 out of 6 replicates. The results of the BPV-3 qPCR did not show any cross reactivity with these closely related species and meet the acceptance criteria with 6 out of 6 replicates not detecting the non-target species/isolates.

Inclusivity demonstrates that the assay can specifically detect BPV-3 target sequence species/isolates. A previously confirmed BPV-3 positive fetal bovine serum sample (confirmation was done by full-length genome sequencing of BPV-3) was extracted followed by BPV-3 qPCR assay. Acceptance criterion for this procedure required that the tested sample show a positive result in at least 5 out of 6 replicates. The results of the BPV-3 qPCR assay did specifically detect BPV-3 DNA and met the acceptance criteria with 6 out of 6 replicates detecting the target isolates.

### Limit of Detection (LOD)

Limit of detection (LOD) is the lowest amount of target sequence which can be detected, but not necessarily quantitated as an exact value. Probit analysis with LOD95% detection limit approach was employed to determine the assay LOD. LOD95% is the number of copies of the target DNA sequence required to ensure 95% probability of detection (POD) in the qPCR assay.

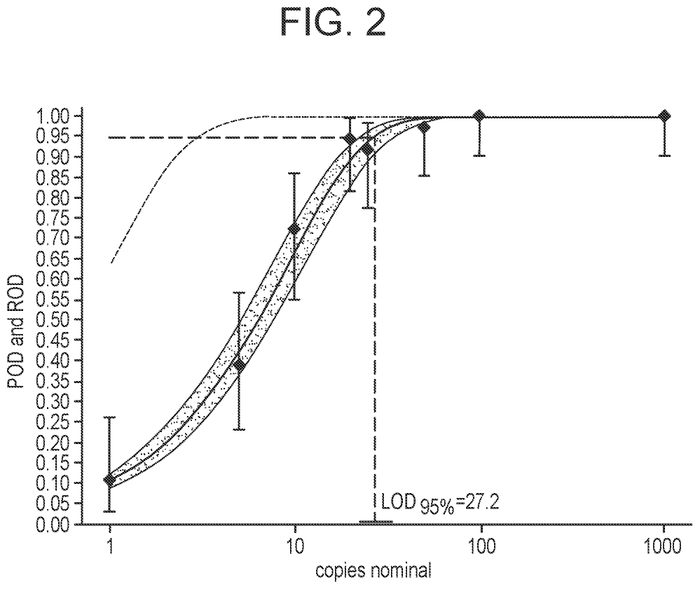

BPV-3 positive control DNA (1000, 100, 50, 25, 20, 10, 5, and 1 genome copies/reaction) was spiked into an exogenous extracted DNA (˜300 ng human genomic DNA) and was tested using the BPV-3 qPCR assay as described in Example 4. Each concentration was tested in 12 PCR replicates to obtain a statistically reliable POD curve from which the LOD95% with 95% confidence interval (CI) was derived. The experiment was repeated by 3 different analysts. Any CT value ≤39.99 was considered a positive signal, and any undetermined CT value (CT=40.00) was considered as a negative signal. The 36 set points for each concentration (gathered from all analysts) were obtained for Probit analysis to achieve a statistically reliable POD curve from which the LOD95% with 95% confidence interval (CI) was derived, See Table 31 and FIG. 2. The Probit analysis results showed that the BPV-3 qPCR assay LOD95% was 27 GC/rxn with a 95% confidence interval of 22 and 34 GC/rxn.

TABLE 31
Result of LOD95% from all experiments

| DNA | # of PCR | # positive |
|---|---|---|
| concentration | replicates from | PCR signal |
| (GC/rxn) | all experiments | CT ≤ 39.99 |
| 1 | 36 | 4 |
| 5 | 36 | 14 |
| 10 | 36 | 26 |
| 20 | 36 | 34 |
| 25 | 36 | 33 |
| 50 | 36 | 35 |
| 100 | 36 | 36 |
| 1000 | 36 | 36 |

### Dynamic Range and Linearity

A suitable level of precision, accuracy and linearity must be demonstrated within the dynamic range of the analytical procedure. To evaluate linearity, the minimum and maximum dynamic (operational) range of the standard curve, a preliminary standard curve was generated and evaluated with concentrations spanning 8 orders of magnitude with the defined copy number of positive control plasmid. Three replicates at each of 1E8, 1E7, 1E6, 1E5, 1E4, 1E3, 1E2, and 1E1 genome copies/reaction, were used in the BVP-3 assay.

A minimum of 6 magnitudes were selected for operational range not only per assay application and expected use but also per acceptable standard curve performance parameters derived from the preliminary testing results. For this procedure, the linearity of the standard curve within 6 chosen magnitudes (including maximum and minimum set points) must meet the acceptance criterions of (i) correlation coefficient (R2) 0.98 and (ii) PCR amplification efficiency within 90-1e10.

Although the dynamic range at seven set points (9E2 to 1E8 genome copies/reaction) met assay acceptance criteria, it was anticipated that using seven set points from E2 to E8 as the minimum and maximum standard range would not be robust. Therefore, an upper magnitude (1E3) for low limit of quantification (LOQ) was selected and the minimum and maximum dynamic range of standard curve was designated to be six data set points between E3 to E8 genome copies/reaction, Table 32.

TABLE 32
Standard curve dynamic range and performance

| | | Correlation | PCR | Acceptance Criteria |
|---|---|---|---|---|
| | | coefficient | amplification | R² ≥ 0.98 |
| Experiment | Dynamic Range | (R²) | efficiency (%) | PCR efficiency: 90-110% |
| 1 | 1E1 to 1E8 | 0.997 | 91.328 | Met |
| | 1E2 to 1E8 | 0.999 | 95.572 | Met |
| | 1E3 to 1E8 | 0.999 | 94.312 | Met |
| 2 | 1E1 to 1E8 | 0.997 | 94.957 | Met |
| | 1E2 to 1E8 | 0.998 | 97.441 | Met |
| | 1E3 to 1E8 | 1.000 | 93.731 | Met |
| 3 | 1E1 to 1E8 | 0.995 | 85.425 | Not Met |
| | 1E2 to 1E8 | 0.998 | 90.827 | Met |
| | 1E3 to 1E8 | 1.000 | 94.630 | Met |

### Accuracy and Precision

The accuracy (trueness) and the precision (repeatability and intermediate precision) of the assay were determined. To determine accuracy and precision two separate sets of positive control plasmids with the defined range and genome copy number for BPV-3 (1E8, 1E7, 1E6, 1E5, 1E4, and 1E3 genome copies/reaction) were prepared and tested in parallel in one 96-well PCR reaction plate performing the BVP-3 assay. One set served as standard reference set points and the other set was used for assay evaluation. This procedure was tested by three different analysts on different days.

For repeatability (intra-assay precision), the mean and standard deviation of the quantity (genome copy of the target sequence/reaction) and % CV (coefficient of variation) of quantity was calculated for each triplicate at each concentration. Acceptance criterion for this procedure was that the % CV of quantity was ≤25%.

For intermediate precision (within-laboratory precision), the mean and standard deviation of quantity and % CV were calculated from all triplicate PCR reactions at all concentrations of the three independent experiments. The acceptance criterion was that the % CV of quantity was ≤30%.

For accuracy, (accounted for the inherent variability in different analysts performing the assay, different detection systems, and the assay being performed on different times/times) the mean quantity (genome copy of target sequence/reaction) was calculated from each triplicate PCR reaction at each concentration (1E8, 1E7, 1E6, 1E5, 1E4, and 1E3). The acceptance criterion was that the % CV of quantity, was calculated for each sample concentration described above. The acceptance criterion was that the mean quantification was within ±30% of the reference value across the entire dynamic range of the assay. Acceptance criterion for this procedure also required that the linearity for each independent experiment must show the correlation coefficient (R²)≥0.98 and PCR amplification efficiency within 90-110%.

The results of the 3 independent runs showed that the assay met the established acceptance criteria for linearity, See Tables 33-36. Repeatability was ≤25%, intermediate precision was ≤25% and accuracy was within ±30%. The Linearity also was within the acceptance criteria with a correlation coefficient (R²)≥0.98 and PCR amplification efficiency within 90-110%.

TABLE 33
Run #1: Repeatability and accuracy of the first experiment met acceptance criteria.

| | | | | | | Acceptance Criteria |
|---|---|---|---|---|---|---|
| | | | Accuracy | | | Accuracy: ±30% of standard |
| | | | Mean of | StdDev | Repeatability | Repeatability: % CV ≤ |
| Sample | | Quantity | Quantity | of | % CV of | 25% R² ≥ 0.98 |
| Name | CT | GC/rxn | (GC/rxn) | quantity | Quantity | PCR efficiency: 90-110% |
| PC_1E8 | 12.465 | 92,025,368 | 93,049,523 | 1,502,632 | 1.61 | Met |
| | 12.421 | 94,774,544 | | | | |
| | 12.460 | 92,348,656 | | | | |
| PC_1E7 | 15.510 | 12,117,075 | 10,908,287 | 1,047,926 | 9.61 | Met |
| | 15.760 | 10,256,219 | | | | |
| | 15.746 | 10,351,568 | | | | |
| PC_1E6 | 19.432 | 889,522 | 895,233 | 7,440 | 0.83 | Met |
| | 19.408 | 903,646 | | | | |
| | 19.427 | 892,530 | | | | |
| PC_1E5 | 22.919 | 87,213 | 88,386 | 1,252 | 1.42 | Met |
| | 22.877 | 89,704 | | | | |
| | 22.902 | 88,239 | | | | |
| PC_1E4 | 26.323 | 9,039 | 9,318 | 287 | 3.08 | Met |
| | 26.280 | 9,302 | | | | |
| | 26.231 | 9,613 | | | | |
| PC_1E3 | 30.033 | 764 | 715 | 47 | 6.64 | Met |
| | 30.141 | 711 | | | | |
| | 30.232 | 669 | | | | |

| | | |
|---|---|---|
| Linearity | Correlation coefficient (R²): 0.999 | Met |
| | PCR amplification efficiency: 94.312% | |

TABLE 34
Run #2: Repeatability and accuracy of the second experiment met acceptance criteria.

| | | | | | | Acceptance Criteria |
|---|---|---|---|---|---|---|
| | | | Accuracy | | | Accuracy: ±30% of standard |
| | | | Mean of | StdDev | Repeatability | Repeatability: % CV ≤ |
| Sample | | Quantity | Quantity | of | % CV of | 25% R² ≥ 0.98 |
| Name | CT | GC/rxn | (GC/rxn) | quantity | Quantity | PCR efficiency: 90-110% |
| PC_1E8 | 12.051 | 95,262,496 | 94,595,301 | 917,893 | 0.97 | Met |
| | 12.079 | 93,548,496 | | | | |
| | 12.056 | 94,974,912 | | | | |
| PC_1E7 | 15.515 | 9,642,861 | 9,573,740 | 64,503 | 0.67 | Met |
| | 15.535 | 9,515,152 | | | | |
| | 15.527 | 9,563,208 | | | | |
| PC_1E6 | 18.915 | 1,017,622 | 1,031,438 | 16,246 | 1.58 | Met |
| | 18.869 | 1,049,335 | | | | |
| | 18.901 | 1,027,357 | | | | |
| PC_1E5 | 22.457 | 97,816 | 94,600 | 2,821 | 2.98 | Met |
| | 22.541 | 92,543 | | | | |
| | 22.526 | 93,441 | | | | |
| PC_1E4 | 25.229 | 15,640* | 9,572 | 20 | 0.21 | Met |
| | 25.970 | 9,586 | | | | |
| | 25.974 | 9,558 | | | | |
| PC_1E3 | 29.679 | 825 | 822 | 4 | 0.45 | Met |
| | 29.691 | 818 | | | | |
| | 29.680 | 824 | | | | |

| | | |
|---|---|---|
| Linearity | Correlation coefficient (R²): 1.000 | Met |
| | PCR amplification efficiency: 93.731% | |

*This value was an outlier per Dixon's Q test and excluded from data analysis.

TABLE 35
Run #3: Repeatability and accuracy of third experiment met acceptance criteria.

| | | | | | | Acceptance Criteria |
|---|---|---|---|---|---|---|
| | | | Accuracy | | | Accuracy: ±30% of standard |
| | | | Mean of | StdDev | Repeatability | Repeatability: % CV ≤ |
| Sample | | Quantity | Quantity | of | % CV of | 25% R² ≥ 0.98 |
| Name | CT | GC/rxn | (GC/rxn) | quantity | Quantity | PCR efficiency: 90-110% |
| PC_1E8 | 11.818 | 117,211,200 | 115,519,208 | 2,229,747 | 1.93 | Met |
| | 11.873 | 112,992,544 | | | | |
| | 11.829 | 116,353,880 | | | | |
| PC_1E7 | 15.668 | 9,084,705 | 12,202,317 | 2,713,306 | 22.24 | Met |
| | 15.073 | 13,492,047 | | | | |
| | 15.014 | 14,030,200 | | | | |
| PC_1E6 | 18.758 | 1,166,552 | 1,279,299 | 119,641 | 9.35 | Met |
| | 18.634 | 1,266,533 | | | | |
| | 18.478 | 1,404,811 | | | | |
| PC_1E5 | 22.277 | 112,593 | 110,984 | 6,333 | 5.71 | Met |
| | 22.397 | 104,002 | | | | |
| | 22.228 | 116,357 | | | | |
| PC_1E4 | 25.871 | 10,344 | 10,570 | 1,468 | 13.89 | Met |
| | 26.043 | 9,227 | | | | |
| | 25.630 | 12,138 | | | | |
| PC_1E3 | 29.156 | 1,167 | 922 | 213 | 23.07 | Met |
| | 29.686 | 820 | | | | |
| | 29.763 | 780 | | | | |

| | | |
|---|---|---|
| Linearity | Correlation coefficient (R²): 0.999 | Met |
| | PCR amplification efficiency: 94.312% | |

TABLE 36
Results of intermediate precision based on 3 separate runs
by different analysts (Runs # 1, 2 and 3).

| | Mean of | | Intermediate | Acceptance Criteria |
|---|---|---|---|---|
| Sample | Quantity | StdDev of | precision % | Intermediate |
| Name | (GC/rxn) | Quantity | CV of quantity | Precision: % CV ≤ 30% |
| PC_1E8 | 101,054,677 | 10,961,469 | 10.85 | Met |
| Run # 1, 2 and 3 | | | | |
| PC_1E7 | 10,894,782 | 1,847,080 | 16.95 | Met |
| Run # 1, 2 and 3 | | | | |
| PC_1E6 | 1,068,657 | 179,151 | 16.76 | Met |
| Run # 1, 2 and 3 | | | | |
| PC_1E5 | 97,990 | 10,706 | 10.93 | Met |
| Run # 1, 2 and 3 | | | | |
| PC_1E4 | 9,851 | 1,003 | 10.18 | Met |
| Run # 1, 2 and 3 | | | | |
| PC_1E3 | 820 | 141 | 17.23 | Met |

### Limit of Quantification

The limit of quantification (LOQ) of the quantitative qPCR assay is the lowest amount of target sequence in a sample which can be quantitatively determined with suitable precision and accuracy. To determine the LOQ, the lowest setpoint (1E3 genome copies/reaction), the mid setpoints (1E4 and 1E6 genome copies/reaction) and upper set point (1E8 genome copies/reaction) of dynamic range were tested. Positive control samples with a concentration at the lowest, middle and upper set points of the dynamic range were prepared and spiked into an exogenous extracted DNA sample (˜300 ng human genomic DNA per reaction, to serve as background sample matrix DNA) and evaluated in 12 replicates performing the BVP-3 assay in the presence of a standard curve. The assay was performed by three analysts on different dates. Precision (repeatability and intermediate precision) and accuracy were determined as described above with the acceptance criteria as described above.

Results of the three independent LOQ experiments were within the established acceptance criteria for repeatability, intermediate precision, and accuracy (Tables 37-39), and linearity (Table 40).

TABLE 37
Run #1: Linearity, repeatability and accuracy of the first LOQ experiment met acceptance criteria.

| | | | | | | Acceptance Criteria |
|---|---|---|---|---|---|---|
| | | | Accuracy | | | Accuracy: ±30% of standard |
| | | | Mean of | StdDev | Repeatability | Repeatability: % CV ≤ |
| Sample | | Quantity | Quantity | of | % CV of | 25% R² ≥ 0.98 |
| Name | CT | GC/rxn | (GC/rxn) | quantity | Quantity | PCR efficiency: 90-110% |
| PC_1E8 | 12.088 | 99,773,728 | 102,123,087 | 5355285 | 5.24 | Met |
| | 12.071 | 100,869,600 | | | | |
| | 12.092 | 99,482,992 | | | | |
| | 12.105 | 98,636,408 | | | | |
| | 12.126 | 97,306,984 | | | | |
| | 12.128 | 97,162,088 | | | | |
| | 11.979 | 107,288,808 | | | | |
| | 12.072 | 100,809,184 | | | | |
| | 12.067 | 101,182,688 | | | | |
| | 12.029 | 103,763,872 | | | | |
| | 12.046 | 102,613,232 | | | | |
| | 11.854 | 116,587,456 | | | | |
| PC_1E6 | 19.376 | 781,088 | 995,748 | 109618 | 11.01 | Met |
| | 19.132 | 918,892 | | | | |
| | 19.104 | 936,234 | | | | |
| | 19.074 | 954,863 | | | | |
| | 19.156 | 904,037 | | | | |
| | 19.144 | 911,503 | | | | |
| | 18.916 | 1,060,829 | | | | |
| | 18.893 | 1,076,882 | | | | |
| | 18.879 | 1,086,972 | | | | |
| | 18.803 | 1,143,163 | | | | |
| | 18.837 | 1,117,681 | | | | |
| | 18.921 | 1,056,834 | | | | |
| PC_1E4 | 25.953 | 9,815 | 10,475 | 2076 | 19.82 | Met |
| | 25.947 | 9,850 | | | | |
| | 26.233 | 8,147 | | | | |
| | 26.162 | 8,539 | | | | |
| | 26.029 | 9,330 | | | | |
| | 25.283 | 15,321 | | | | |
| | 25.849 | 10,513 | | | | |
| | 25.888 | 10,248 | | | | |
| | 26.029 | 9,329 | | | | |
| | 26.034 | 9,297 | | | | |
| | 25.521 | 13,078 | | | | |
| | 25.621 | 12,237 | | | | |
| PC_1E3 | 29.313 | 1,049 | 1,162 | 274 | 23.56 | Met |
| | 28.819 | 1,457 | | | | |
| | 29.434 | 968 | | | | |
| | 29.798 | 760 | | | | |
| | 29.571 | 883 | | | | |
| | 29.050 | 1,249 | | | | |
| | 29.420 | 977 | | | | |
| | 28.775 | 1,500 | | | | |
| | 28.688 | 1,590 | | | | |
| | 28.847 | 1,430 | | | | |
| | 29.250 | 1,094 | | | | |
| | 29.407 | 985 | | | | |

| | | |
|---|---|---|
| Linearity | Correlation coefficient (R²): 0.999 | Met |
| | PCR amplification efficiency: 94.544% | |

TABLE 38
Run #2: Linearity, repeatability and accuracy of the second LOQ experiment met acceptance criteria.

| | | | | | | Acceptance Criteria |
|---|---|---|---|---|---|---|
| | | | Accuracy | | | Accuracy: ±30% of standard |
| | | | Mean of | StdDev | Repeatability | Repeatability: % CV ≤ |
| Sample | | Quantity | Quantity | of | % CV of | 25% R² ≥ 0.98 |
| Name | CT | GC/rxn | (GC/rxn) | quantity | Quantity | PCR efficiency: 90-110% |
| PC_1E8 | 12.095 | 95,058,592 | 93,650,034 | 1,438,673 | 1.54 | Met |
| | 12.120 | 93,524,168 | | | | |
| | 12.115 | 93,828,104 | | | | |
| | 12.118 | 93,629,304 | | | | |
| | 12.112 | 94,045,632 | | | | |
| | 12.154 | 91,458,488 | | | | |
| | 12.109 | 94,209,456 | | | | |
| | 12.100 | 94,746,560 | | | | |
| | 12.094 | 95,136,672 | | | | |
| | 12.135 | 92,587,520 | | | | |
| | 12.168 | 90,615,920 | | | | |
| | 12.097 | 94,959,992 | | | | |
| PC_1E6 | 19.256 | 838,090 | 854,259 | 24,034 | 2.81 | Met |
| | 19.177 | 882,874 | | | | |
| | 19.225 | 855,340 | | | | |
| | 19.217 | 859,674 | | | | |
| | 19.237 | 848,636 | | | | |
| | 19.230 | 852,521 | | | | |
| | 19.295 | 816,498 | | | | |
| | 19.207 | 865,467 | | | | |
| | 19.261 | 835,148 | | | | |
| | 19.132 | 909,394 | | | | |
| | 19.253 | 839,838 | | | | |
| | 19.239 | 847,628 | | | | |
| PC_1E4 | 26.141 | 8,864 | 8,691 | 430 | 4.95 | Met |
| | 26.052 | 9,400 | | | | |
| | 26.174 | 8,670 | | | | |
| | 26.226 | 8,378 | | | | |
| | 26.154 | 8,787 | | | | |
| | 26.174 | 8,672 | | | | |
| | 26.194 | 8,560 | | | | |
| | 26.314 | 7,907 | | | | |
| | 26.197 | 8,543 | | | | |
| | 26.265 | 8,168 | | | | |
| | 26.083 | 9,209 | | | | |
| | 26.095 | 9,138 | | | | |
| PC_1E3 | 29.738 | 823 | 828 | 75 | 9.03 | Met |
| | 29.680 | 855 | | | | |
| | 29.774 | 804 | | | | |
| | 29.703 | 842 | | | | |
| | 29.747 | 818 | | | | |
| | 29.647 | 874 | | | | |
| | 29.528 | 946 | | | | |
| | 29.952 | 715 | | | | |
| | 29.871 | 754 | | | | |
| | 29.895 | 742 | | | | |
| | 29.503 | 961 | | | | |
| | 29.770 | 806 | | | | |

| | | |
|---|---|---|
| Linearity | Correlation coefficient (R²): 1.000 | Met |
| | PCR amplification efficiency: 93.621% | |

TABLE 39
Run #3: Linearity, repeatability and accuracy of the third LOQ experiment met acceptance criteria.

| | | | | | | Acceptance Criteria |
|---|---|---|---|---|---|---|
| | | | Accuracy | | | Accuracy: ±30% of standard |
| | | | Mean of | StdDev | Repeatability | Repeatability: % CV ≤ |
| Sample | | Quantity | Quantity | of | % CV of | 25% R² ≥ 0.98 |
| Name | CT | GC/rxn | (GC/rxn) | quantity | Quantity | PCR efficiency: 90-110% |
| PC_1E8 | 11.987 | 87,553,632 | 86,941,879 | 1,185,070 | 1.36 | Met |
| | 12.009 | 86,286,488 | | | | |
| | 12.016 | 85,904,016 | | | | |
| | 11.983 | 87,763,088 | | | | |
| | 11.996 | 87,037,040 | | | | |
| | 11.987 | 87,556,344 | | | | |
| | 11.976 | 88,172,408 | | | | |
| | 12.015 | 85,993,864 | | | | |
| | 11.958 | 89,230,456 | | | | |
| | 12.034 | 84,890,344 | | | | |
| | 12.012 | 86,159,392 | | | | |
| | 12.001 | 86,755,472 | | | | |
| PC_1E6 | 19.003 | 919,457 | 869,476 | 39,354 | 4.53 | Met |
| | 19.096 | 865,214 | | | | |
| | 19.106 | 859,918 | | | | |
| | 19.088 | 869,696 | | | | |
| | 19.140 | 841,225 | | | | |
| | 19.095 | 866,096 | | | | |
| | 19.091 | 868,491 | | | | |
| | 19.138 | 842,210 | | | | |
| | 19.174 | 822,775 | | | | |
| | 18.935 | 960,591 | | | | |
| | 19.168 | 826,054 | | | | |
| | 19.049 | 891,982 | | | | |
| PC_1E4 | 26.388 | 7,597 | 9,014 | 1148 | 12.73 | Met |
| | 26.298 | 8,053 | | | | |
| | 26.240 | 8,365 | | | | |
| | 26.272 | 8,188 | | | | |
| | 26.242 | 8,350 | | | | |
| | 25.987 | 9,854 | | | | |
| | 25.865 | 10,671 | | | | |
| | 24.184 | 31,781* | | | | |
| | 26.077 | 9,298 | | | | |
| | 25.823 | 10,964 | | | | |
| | 25.211 | 16,316* | | | | |
| | 26.162 | 8,795 | | | | |
| PC_1E3 | 30.058 | 701 | 732 | 39 | 5.33 | Met |
| | 29.949 | 752 | | | | |
| | 30.100 | 682 | | | | |
| | 30.149 | 660 | | | | |
| | 29.909 | 772 | | | | |
| | 29.909 | 772 | | | | |
| | 29.079 | 1,323* | | | | |
| | 29.114 | 1,294* | | | | |
| | 30.011 | 722 | | | | |
| | 29.948 | 753 | | | | |
| | 29.958 | 748 | | | | |
| | 29.935 | 759 | | | | |

| | | |
|---|---|---|
| Linearity | Correlation coefficient (R²): 1.000 | Met |
| | PCR amplification efficiency: 91.443% | |

*Values were outliers per IQR test and excluded from data analysis.

TABLE 40
Results of intermediate precision based on 3 independent
LOQ experiments/runs met acceptance criteria.

| | Mean of | | Intermediate | Acceptance Criteria |
|---|---|---|---|---|
| Sample | Quantity | StdDev of | precision % | Intermediate |
| Name | (GC/rxn) | Quantity | CV of Quantity | Precision: % CV ≤ 30% |
| PC_1E8 | 94,238,333 | 7056365 | 7.49 | Met |
| Run # 1, 2 and 3 | | | | |
| PC_1E6 | 906,494 | 92636 | 10.22 | Met |
| Run # 1, 2 and 3 | | | | |
| PC_1E4 | 9,416 | 1583 | 16.81 | Met |
| Run # 1, 2 and 3 | | | | |
| PC_1E3 | 918 | 250 | 27.19 | Met |
| Run # 1, 2 and 3 | | | | |

### Robustness

To determine the robustness of the assay, changes in critical reagent concentrations of up to 20% were evaluated according to the method of Youden and Steiner (supra). Several critical qPCR factors were selected and subjected to slight changes, Table 41. A BPV-3 positive fetal bovine serum test sample was extracted and tested in 12 replicates using the optimized and changed conditions in the BPV-3 assay in parallel with a standard curve.

TABLE 41
qPCR factors tested

| | Combination matrix condition # | | | | |
|---|---|---|---|---|---|
| | Optimized | | | | |
| PCR Factor | condition | 1 | 2 | 3 | 4 |
| Applied Biosystems | Lot A | Lot A | Lot A | Lot B | Lot B |
| qPCR Master Mix | | | | | |
| BPV-3 primer/probe | Lot A | Lot A | Lot B | Lot B | Lot A |
| BPV-3 primer/probe | 1x | 1.2x | 0.8x | 1.2x | 0.8x |
| concentration | | | | | |
| PCR Vol. (μL) | 30 | 25 | 25 | 35 | 35 |

The acceptance criterion for this procedure required that the response obtained from any robustness condition with respect to the applied small changes meet the established assay acceptance criteria. Acceptance criterion for this procedure included precision, the CV of quantity for repeatability and intermediate precision was ≤25% as described above. For accuracy, the mean of quantity of the combination matrix conditions (conditions 1 to 4) was ±30% of the mean of quantity of the optimized condition.

Results of robustness study showed that slight changes in the qPCR critical reagents were tolerable and the robustness results met the precision and accuracy (118,100±35,430) acceptance criteria when compared to the optimized condition, Table 42.

TABLE 42
Repeatability and accuracy of the robustness experiment met acceptance criteria

| | | | | | | Acceptance Criteria |
|---|---|---|---|---|---|---|
| | | | | | | Accuracy: ±30% of |
| | | | Accuracy | | | optimized cond. |
| | | | Mean of | StdDev | Repeatability | Repeatability: % CV ≤ |
| Sample | | Quantity | Quantity | of | % CV of | 25% R² ≥ 0.98 |
| Name | CT | GC/rxn | (GC/rxn) | quantity | Quantity | PCR efficiency: 90-110% |
| Optimized | 21.342 | 119,550 | 118,100 | 3359 | 2.84 | Met |
| condition | 21.347 | 119,099 | | | | |
| | 21.345 | 119,303 | | | | |
| | 21.400 | 114,637 | | | | |
| | 21.357 | 118,231 | | | | |
| | 21.402 | 114,449 | | | | |
| | 21.388 | 115,630 | | | | |
| | 21.413 | 113,586 | | | | |
| | 21.381 | 116,243 | | | | |
| | 21.288 | 124,286 | | | | |
| | 21.301 | 123,106 | | | | |
| | 21.347 | 119,083 | | | | |
| Robustness | 21.198 | 132,632 | 129,070 | 3071 | 2.38 | Met |
| condition | 21.229 | 129,739 | | | | |
| (1) | 21.261 | 126,719 | | | | |
| | 21.248 | 127,949 | | | | |
| | 21.265 | 126,411 | | | | |
| | 21.283 | 124,755 | | | | |
| | 21.274 | 125,541 | | | | |
| | 21.247 | 128,075 | | | | |
| | 21.216 | 130,993 | | | | |
| | 21.204 | 132,088 | | | | |
| | 21.177 | 134,715 | | | | |
| | 21.234 | 129,221 | | | | |
| Robustness | 21.582 | 100,455 | 97,559 | 4251 | 4.36 | Met |
| condition | 21.633 | 96,803 | | | | |
| (2) | 21.600 | 99,194 | | | | |
| | 21.630 | 97,070 | | | | |
| | 21.594 | 99,617 | | | | |
| | 21.697 | 92,446 | | | | |
| | 21.725 | 90,595 | | | | |
| | 21.697 | 92,449 | | | | |
| | 21.651 | 95,582 | | | | |
| | 21.596 | 99,439 | | | | |
| | 21.524 | 104,787 | | | | |
| | 21.558 | 102,275 | | | | |
| Robustness | 21.230 | 129,609 | 124,259 | 5180 | 4.17 | Met |
| condition | 21.248 | 127,920 | | | | |
| (3) | 21.283 | 124,744 | | | | |
| | 21.278 | 125,207 | | | | |
| | 21.369 | 117,245 | | | | |
| | 21.324 | 121,089 | | | | |
| | 21.351 | 118,724 | | | | |
| | 21.349 | 118,902 | | | | |
| | 21.357 | 118,260 | | | | |
| | 21.246 | 128,132 | | | | |
| | 21.227 | 129,892 | | | | |
| | 21.211 | 131,381 | | | | |
| Robustness | 21.513 | 105,590 | 101,792 | 4841 | 4.76 | Met |
| condition | 21.547 | 103,024 | | | | |
| (4) | 21.526 | 104,609 | | | | |
| | 21.575 | 100,966 | | | | |
| | 21.618 | 97,889 | | | | |
| | 21.633 | 96,866 | | | | |
| | 21.648 | 95,779 | | | | |
| | 21.626 | 97,330 | | | | |
| | 21.621 | 97,667 | | | | |
| | 21.544 | 103,268 | | | | |
| | 21.490 | 107,378 | | | | |
| | 21.443 | 111,135 | | | | |

| | | |
|---|---|---|
| Standard | Correlation coefficient (R²): 0.987 | Met |
| | PCR amplification efficiency: 106.209% | |

### Verification

Assay system suitability and assay acceptance criteria were defined to verify assay performance per qualification protocol, see Table 44. To demonstrate that the assay performs as expected a test sample containing target sequence (Sample A) and a test sample with no target sequence (Sample B) were subjected to DNA extraction followed by BPV-3 qPCR. The verification was tested 3 times by different analysts on different days. Assay controls, system suitability and assay acceptance criteria and reporting and interpretation of test results are shown in Tables 43-44.

TABLE 43
Defined system suitability and assay acceptance criteria for BPV-3 qPCR assay

| | Control | Valid assay acceptance criteria |
|---|---|---|
| BPV-3 Target | | |
| Sequence | | |
| BPV-3 | NTC | A minimum of 2 out of 3 reactions must show no amplification signal, |
| | NEC | CT = 40.00 (undetermined) and the obtained mean of GC quantity per |
| | | reaction (if any) shall be less than the assay LOD95% (27 GC/reaction). |
| BPV-3 | PEC | A minimum of 2 out of 3 reactions must show amplification signal of |
| | | CT < 40.00, and the obtained mean GC of quantity per reaction shall be |
| | | greater than assay LOQ (1E3 GC/rxn). |
| BPV-3 | PC_2E4 | A minimum of 2 out of 3 reactions must show amplification signal of |
| | PC_2E5 | CT < 40.00, and the obtained mean GC of quantity per reaction must |
| | PC_2E7 | meet the established assay accuracy criteria, +30% of PC reference value: |
| | | PC_2E7 acceptance accuracy range: 1.4E7 to 2.6E8 GC/rxn |
| | | PC_2E5 acceptance accuracy range: 1.4E5 to 2.6E6 GC/rxn |
| | | PC_2E4 acceptance accuracy range: 1.4E4 to 2.6E4 GC/rxn |
| BPV-3 | INH or | A minimum of 2 out of 3 reactions must show amplification signal of |
| | (+S) | CT < 40.00, and the obtained mean GC of quantity per reaction shall be |
| | | within one magnitude of the original (2E4) spiked concentration level; |
| | | the INH mean GC of quantity should be within 2E3 to 2E5 GC/reaction. |
| | | NOTE: INH control is irrelevant and not applicable if test sample is |
| | | positive with BPV-3 with mean GC of quantity of equal or greater than |
| | | 2E4 per reaction. |
| BPV-3 | ST | Correlation coefficient (R2) ≥ 0.98 |
| | | PCR amplification efficiency within 90 to 110%. |
| IPC Target | | |
| Sequence | | |
| IPC | NEC | A minimum of 2 out of 3 reactions must show no amplification signal, |
| | | CT = 40.00 (undetermined). |
| IPC | PEC | A minimum of 2 out of 3 reactions must show amplification signal of |
| | | CT < 40.00. |
| IPC | Test | A minimum of 2 out of 3 reactions must show no amplification signal, |
| | Sample | CT = 40.00 (undetermined). |

TABLE 44
Reporting and interpretation of data.

| Test sample (BPV-3 GC/rxn) | Report | Interpretation |
|---|---|---|
| BPV-3 CT value is | BPV-3 Not Detected | Target sequence not detected or |
| Undetermined (CT = 40.00) | | detected at a level below the |
| indicating the absence of BPV-3 | | LOD95% of the assay. |
| or the mean of the BPV-3 GC/rxn | | |
| is equal or less than assay | | |
| LOD95% (27 GC/rxn) | | |
| Mean GC/rxn of BPV-3 is | BPV-3 Detected | Target sequence is detected, but |
| less than assay LOQ (1E3) and | (provide quantity as Log10 of | at a level below the LOQ of the |
| greater than assay LOD95% | mean ± StdDev per mL) | assay; not accurately quantifiable. |
| (27 GC/rxn) | | |
| Mean of BPV-3 GC/rxn is | BPV-3 Detected | Target sequence is detected, and |
| within the dynamic range of the | (provide quantity as Log10 of | quantification is accurate and |
| assay (1E3 to 1E8 GC/rxn) | mean ± StdDev per mL) | within the linear range of the assay. |
| Mean GC/rxn of BPV-3 is | BPV-3 Detected | Target sequence is detected and is |
| greater than upper set point | (provide quantity as Log10 of | greater than the upper dynamic |
| (1E8) of assay dynamic range | mean ± StdDev per mL) | range of the assay. |
| | Alternatively, the quantity may | The assay can be repeated with |
| | be reported as greater than the | different dilution factors to allow |
| | upper level of the dynamic | for quantification within |
| | range of the assay. | operational range. |

### Results of the Verification Study

Tables 45, 47 and 49 present the results of assay system suitability and assay controls for the three independent experiments. All three independent experiments/runs met the established system suitability and assay control acceptance criteria as defined herein. Tables 45, 47 and 49 present test samples results (Sample A: previously confirmed positive sample, and Sample B: previously confirmed negative sample). Results of verification study demonstrated that BPV-3 was not detected in the negative sample (Sample B) among all three independent experiments/runs and BPV-3 could be detected and quantified in the positive sample (Sample A) through all three independent experiments/runs.

TABLE 45
Verification Run #1 met acceptance criteria
for assay system suitability and assay controls.

| | | | | Mean of | StdDev | |
|---|---|---|---|---|---|---|
| | | | Quantity | quantity | of | Acceptance |
| Target | Control | CT | GC/rxn | (GC/rxn) | quantity | Criteria |
| BPV-3 | PC_2E7 | 14.31 | 18,326,098 | 18,401,424 | 67,871 | Met |
| v3 PP | | 14.30 | 18,420,354 | | | |
| | | 14.29 | 18,457,820 | | | |
| | PC_2E5 | 21.49 | 168,383 | 167,435 | 3,214 | Met |
| | | 21.53 | 163,853 | | | |
| | | 21.47 | 170,069 | | | |
| | PC_2E4 | 24.77 | 19,824 | 19,485 | 359 | Met |
| | | 24.79 | 19,521 | | | |
| | | 24.82 | 19,109 | | | |
| | Sample | 24.97 | 17,393 | 17,452 | 53 | Met |
| | B + S | 24.96 | 17,468 | | | |
| | | 24.96 | 17,495 | | | |
| | Sample | 20.99 | 232,852 | 225,642 | 6,254 | N/A |
| | A + S | 21.07 | 221,700 | | | |
| | | 21.06 | 222,372 | | | |
| | PEC | 27.67 | 2,981 | 2,935 | 124 | Met |
| | | 27.65 | 3,029 | | | |
| | | 27.77 | 2,794 | | | |
| | NEC | 40.00 | 0 | 0 | 0 | Met |
| | | 40.00 | 0 | 0 | 0 | |
| | | 40.00 | 0 | 0 | 0 | |
| | NTC | 40.00 | 0 | 0 | 0 | Met |
| | | 40.00 | 0 | 0 | 0 | |
| | | 40.00 | 0 | 0 | 0 | |
| IPC | Sample B | 40.00 | N/A | | | Met |
| v2 PP | | 40.00 | | | | |
| | | 40.00 | | | | |
| | Sample A | 40.00 | | | | Met |
| | | 40.00 | | | | |
| | | 40.00 | | | | |
| | PEC | 27.40 | | | | Met |
| | | 27.35 | | | | |
| | | 27.42 | | | | |
| | NEC | 40.00 | | | | Met |
| | | 40.00 | | | | |
| | | 40.00 | | | | |

| | | | |
|---|---|---|---|
| BPV-3 | Standard | Correlation coefficient (R²): 1.000 | Met |
| v3 PP | | PCR amplification efficiency: 92.08% | |

TABLE 46
Verification Run #1 sample results demonstrated that Sample
B was negative for BPV-3, and Sample A was positive for BPV-3
and the quantity within the dynamic range of the assay

| | | | | Mean of | StdDev | | Quantity |
|---|---|---|---|---|---|---|---|
| | Test | | Quantity | quantity | of | BPV-3 | Log10 GC/mL |
| Target | Sample | CT | GC/rxn | (GC/rxn) | quantity | detected? | (Mean ± StdDev) |
| BPV-3 | Sample B | 40.00 | 0 | 0 | 0 | No | N/A |
| v3 PP | | 40.00 | 0 | | | | |
| | | 40.00 | 0 | | | | |
| | Sample A | 21.21 | 202,610 | 206,757 | 3,828 | Yes | 7.01 ± 0.01 |
| | | 21.15 | 210,154 | | | | |
| | | 21.17 | 207,507 | | | | |

BPV-3 was detected in Sample A at concentration of Log₁₀ 7.01±0.01 genome copies/mL (Mean±StdDev). No BPV-3 was detected in Sample B.

TABLE 47
Verification Run #2 met acceptance criteria
for assay system suitability and assay controls

| | | | | Mean of | | Acceptance |
|---|---|---|---|---|---|---|
| | | | Quantity | quantity | StdDev of | Criteria Refer |
| Target | Control | CT | GC/rxn | (GC/rxn) | quantity | to Table 19 |
| BPV-3 | PC_2E7 | 14.12 | 20,329,452 | 20,227,899 | 112,064 | Met |
| v3 PP | | 14.12 | 20,246,576 | | | |
| | | 14.13 | 20,107,670 | | | |
| | PC_2E5 | 21.12 | 214,577 | 208,974 | 11,572 | Met |
| | | 21.26 | 195,666 | | | |
| | | 21.11 | 216,678 | | | |
| | PC_2E4 | 24.72 | 20,728 | 20,763 | 497 | Met |
| | | 24.75 | 20,285 | | | |
| | | 24.68 | 21,277 | | | |
| | Sample | 24.86 | 18,896 | 18,752 | 1,277 | Met |
| | B + S | 24.78 | 19,951 | | | |
| | | 24.99 | 17,409 | | | |
| | Sample | 21.13 | 214,134 | 213,831 | 263 | N/A |
| | A + S | 21.13 | 213,692 | | | |
| | | 21.13 | 213,667 | | | |
| | PEC | 27.67 | 3,044 | 2,874 | 148 | Met |
| | | 27.81 | 2,777 | | | |
| | | 27.80 | 2,799 | | | |
| | NEC | 40.00 | 0 | 0 | 0 | Met |
| | | 40.00 | 0 | 0 | 0 | |
| | | 40.00 | 0 | 0 | 0 | |
| | NTC | 40.00 | 0 | 0 | 0 | Met |
| | | 40.00 | 0 | 0 | 0 | |
| | | 40.00 | 0 | 0 | 0 | |
| IPC | Sample B | 40.00 | N/A | | | Met |
| v2 PP | | 40.00 | | | | |
| | | 40.00 | | | | |
| | Sample A | 40.00 | | | | Met |
| | | 40.00 | | | | |
| | | 40.00 | | | | |
| | PEC | 27.52 | | | | Met |
| | | 27.66 | | | | |
| | | 27.57 | | | | |
| | NEC | 40.00 | | | | Met |
| | | 40.00 | | | | |
| | | 40.00 | | | | |

| | | | |
|---|---|---|---|
| BPV-3 | Standard | Correlation coefficient (R²): 1.000 | Met |
| v3 PP | | PCR amplification efficiency: 91.50% | |

TABLE 48
Verification Run #2 sample results demonstrated that Sample
B was negative for BPV-3, and Sample A was positive for BPV-3
and the quantity within the dynamic range of the assay

| | | | | Mean of | StdDev | | Quantity |
|---|---|---|---|---|---|---|---|
| | Test | | Quantity | quantity | of | BPV-3 | Log10 GC/mL |
| Target | Sample | CT | GC/rxn | (GC/rxn) | quantity | detected? | (Mean ± StdDev) |
| BPV-3 | Sample B | 40.00 | 0 | 0 | 0 | No | N/A |
| v3 PP | | 40.00 | 0 | | | | |
| | | 40.00 | 0 | | | | |
| | Sample A | 21.15 | 210,494 | 218,073 | 6,591 | Yes | 7.04 ± 0.01 |
| | | 21.07 | 222,461 | | | | |
| | | 21.08 | 221,264 | | | | |

BPV-3 was detected in Sample A at concentration of Log₁₀ 7.04±0.01 genome copies/mL (Mean±StdDev). No BPV-3 was detected in Sample B.

TABLE 49
Verification Run #3 met acceptance criteria
for assay system suitability and assay controls.

| | | | | Mean of | StdDev | Acceptance |
|---|---|---|---|---|---|---|
| | | | Quantity | quantity | of | Criteria Refer |
| Target | Control | CT | GC/rxn | (GC/rxn) | quantity | to Table 19 |
| BPV-3 | PC_2E7 | 13.92 | 23,374,386 | 24,090,493 | 835,483 | Met |
| v3 PP | | 13.82 | 25,008,390 | | | |
| | | 13.89 | 23,888,704 | | | |
| | PC_2E5 | 21.05 | 223,478 | 226,814 | 5,914 | Met |
| | | 21.05 | 223,321 | | | |
| | | 20.98 | 233,642 | | | |
| | PC_2E4 | 24.79 | 19,401 | 19,309 | 213 | Met |
| | | 24.82 | 19,065 | | | |
| | | 24.79 | 19,459 | | | |
| | Sample | 24.69 | 20,650 | 20,570 | 213 | Met |
| | B + S | 24.69 | 20,732 | | | |
| | | 24.72 | 20,329 | | | |
| | Sample | 21.12 | 212,975 | 211,790 | 2,873 | N/A |
| | A + S | 21.15 | 208,515 | | | |
| | | 21.11 | 213,881 | | | |
| | PEC | 27.46 | 3,390 | 3,292 | 140 | Met |
| | | 27.48 | 3,355 | | | |
| | | 27.58 | 3,132 | | | |
| | NEC | 40.00 | 0 | 0 | 0 | Met |
| | | 40.00 | 0 | 0 | 0 | |
| | | 40.00 | 0 | 0 | 0 | |
| | NTC | 40.00 | 0 | 0 | 0 | Met |
| | | 40.00 | 0 | 0 | 0 | |
| | | 40.00 | 0 | 0 | 0 | |
| IPC | Sample B | 40.00 | N/A | | | Met |
| v2 PP | | 40.00 | | | | |
| | | 40.00 | | | | |
| | Sample A | 40.00 | | | | Met |
| | | 40.00 | | | | |
| | | 40.00 | | | | |
| | PEC | 27.51 | | | | Met |
| | | 27.52 | | | | |
| | | 27.57 | | | | |
| | NEC | 40.00 | | | | Met |
| | | 40.00 | | | | |
| | | 40.00 | | | | |

| | | | |
|---|---|---|---|
| BPV-3 | Standard | Correlation coefficient (R²): 1.000 | Met |
| v3 PP | | PCR amplification efficiency: 92.114 | |

TABLE 50
Verification Run #3 sample results demonstrated that Sample
B is negative for BPV-3, and Sample A is positive for BPV-3
and the quantity within the dynamic range of the assay

| | | | | Mean of | StdDev | | Quantity |
|---|---|---|---|---|---|---|---|
| | Test | | Quantity | quantity | of | BPV-3 | Log10 GC/mL |
| Target | Sample | CT | GC/rxn | (GC/rxn) | quantity | detected? | (Mean ± StdDev) |
| BPV-3 | Sample B | 40.00 | 0 | 0 | 0 | No | N/A |
| v3 PP | | 40.00 | 0 | | | | |
| | | 40.00 | 0 | | | | |
| | Sample A | 21.09 | 217,541 | 220,903 | 4,909 | Yes | 7.04 ± 0.01 |
| | | 21.03 | 226,536 | | | | |
| | | 21.08 | 218,632 | | | | |

BPV-3 is detected in Sample A at concentration of Log₁₀ 7.04±0.01 genome copies/mL (Mean±StdDev). No BPV-3 is detected in Sample B.

Table 51 presents the established BPV-3 assay qualification criteria. The qualified quantitative BPV-3 assay allows for quantification of 1E3 to 1E8 BPV-3 genome copies per PCR reaction.

TABLE 51
BPV-3 assay qualified criteria

| Parameter | Qualified Criteria |
|---|---|
| Dynamic Range | 1E3 to 1E8 GC/rxn |
| Lower limit of quantification | 1E3 GC/rxn |
| Upper limit of quantification | 1E8 GC/rxn |
| Limit of detection (LOD95%) | 27 GC/rxn with a 95% confidence |
| | interval of 22 and 34 GC/rxn |

Tested Samples A and B met required qualification specifications for a quantitative assay including specificity, limit of detection, limit of quantification, linearity, precision, accuracy and robustness.

All tested samples (Samples A and B) met the proposed system suitability and assay acceptance criteria. The quantification level between three independent runs showed similar results (Tables 45, 47 and 49) indicating the accurate, precise and robust performance of the qualified assay. The quantitative BPV-3 assay and associated qualification data demonstrated that the BPV-3 quantitative real-time PCR assay allowed detection and quantification of BPV-3 DNA in test sample.

What is claimed is:

1. A composition comprising oligonucleotides selected from the groups consisting of
a) oligonucleotides having the nucleic acid sequence of SEQ ID NO:1, SEQ ID NO:2, and SEQ ID NO:3;
b) oligonucleotides having the nucleic acid sequence of SEQ ID NO:4, SEQ ID NO:5, and SEQ ID NO:6;
c) oligonucleotides having the nucleic acid sequence of SEQ ID NO:7, SEQ ID NO:8, and SEQ ID NO:9;
d) oligonucleotides having the nucleic acid sequence of SEQ ID NO:10, SEQ ID NO: 11, and SEQ ID NO:12;
e) oligonucleotides having the nucleic acid sequence of SEQ ID NO:13, SEQ ID NO:14, and SEQ ID NO: 15;
f) oligonucleotides having the nucleic acid sequence of SEQ ID NO:20, SEQ ID NO:21, and SEQ ID NO:22;
g) oligonucleotides having the nucleic acid sequence of SEQ ID NO:23, SEQ ID NO:24, and SEQ ID NO:25; and
h) oligonucleotides having the nucleic acid sequence of SEQ ID NO:26, SEQ ID NO:27, and SEQ ID NO:28.

2. A composition according to claim 1, wherein the composition optionally includes a second composition comprises oligonucleotides having the sequence of SEQ ID NO: 16, SEQ ID NO: 17, and SEQ ID NO: 18.

3. A composition according to claim 1, wherein the composition comprises oligonucleotides having the nucleic acid sequence of SEQ ID NO:7, SEQ ID NO:8, and SEQ ID NO:9.

4. A composition according to claim 1, wherein the composition comprises a first composition comprises oligonucleotides having the nucleic acid sequence of SEQ ID NO:7, SEQ ID NO:8, and SEQ ID NO:9 and a second composition comprises oligonucleotides having the sequence of SEQ ID NO: 16, SEQ ID NO: 17, and SEQ ID NO: 18.

5. A reagent for detecting Bovine parvovirus 3 (BPV-3) genomic DNA in the extracted DNA of a test sample selected from the group consisting of
a) a primer probe combination having the nucleic acid sequences of SEQ ID NO:1, SEQ ID NO:2, and SEQ ID NO:3;
b) a primer probe combination having the nucleic acid sequences of SEQ ID NO:4, SEQ ID NO:5, and SEQ ID NO:6;
c) a primer probe combination having the nucleic acid sequences of SEQ ID NO:7, SEQ ID NO:8, and SEQ ID NO:9;
d) a primer probe combination having the nucleic acid sequences of SEQ ID NO:10, SEQ ID NO:11, and SEQ ID NO:12;
e) a primer probe combination having the nucleic acid sequences of SEQ ID NO: 13, SEQ ID NO: 14, and SEQ ID NO:15;
f) a primer probe combination having the nucleic acid sequences of SEQ ID NO:20, SEQ ID NO:21, and SEQ ID NO:22;
g) a primer probe combination having the nucleic acid sequences of SEQ ID NO:23, SEQ ID NO:24, and SEQ ID NO:25; and
h) a primer probe combination having the nucleic acid sequences of SEQ ID NO:26, SEQ ID NO:27, and SEQ ID NO:28.

6. The reagent according to claim 5, wherein SEQ ID NO: 3, SEQ ID NO:6, SEQ ID NO:9, SEQ ID NO:12, SEQ ID NO:15, SEQ ID NO:22, SEQ ID NO:25; or SEQ ID NO:28 have a fluorescent reporter dye and/or a non-fluorescent quencher.

7. The reagent according to claim 5, wherein one or more of SEQ ID NO: 3, SEQ ID NO:6, SEQ ID NO:9, SEQ ID NO: 12, SEQ ID NO:15, SEQ ID NO:22, SEQ ID NO:25; or SEQ ID NO:28 have a fluorescent reporter dye 6-carboxyfluorescein (FAM) at the 5′ end and/or either a Minor Groove Binder non-fluorescence quencher (MGB-NFQ) or ZEN-IB and Iowa Black Fluorescence quencher (IBFQ) at the 3′ end.

8. The reagent according to claim 5, wherein the primer probe combination detects DNA encoding the structural capsid (VP) protein and/or the non-structural (NS) protein of Bovine parvovirus 3 in the test sample.

9. The reagent according to claim 5 in combination with an internal positive control primer combination.

10. The reagent according to claim 5, comprising SEQ ID NO:7, SEQ ID NO:8, and SEQ ID NO:9.

11. A reagent for use as an internal positive control primer probe combination in an assay for detecting Bovine parvovirus 3 (BPV-3) genomic DNA in the extracted DNA of a test sample comprising a primer probe combination.

12. The reagent according to claim 11, wherein the primer probe combination has the nucleic acid sequences of SEQ ID NO: 16, SEQ ID NO: 17, and SEQ ID NO: 18.

13. The reagent according to claim 11, wherein SEQ ID NO: 18 has a fluorescent reporter dye and/or a non-fluorescent quencher.

14. The reagent according to claim 13, wherein SEQ ID NO: 18 has a fluorescent reporter dye, 2′-chloro-7′phenyl-1,4-dichloro-6-carboxy-fluorescein (VIC) at the 5′ end and a Minor Groove Binder non-fluorescent quencher (MGB-NFQ) at the 3′ end.

15. A primer probe combination for detecting Bovine parvovirus 3 genomic DNA in the extracted DNA of a test sample in combination with an internal positive control primer probe combination for detecting Bovine parvovirus 3 (BPV-3) genomic DNA.

16. A primer probe combination for detecting Bovine parvovirus 3 genomic DNA according to claim 15 wherein the primer probe combination is selected from the groups consisting of
a) a primer probe combination having the nucleic acid sequences of SEQ ID NO:1, SEQ ID NO:2, and SEQ ID NO:3;
b) a primer probe combination having the nucleic acid sequences of SEQ ID NO:4, SEQ ID NO:5, and SEQ ID NO:6;
c) a primer probe combination having the nucleic acid sequences of SEQ ID NO:7, SEQ ID NO:8, and SEQ ID NO:9;
d) a primer probe combination having the nucleic acid sequences of SEQ ID NO: 10, SEQ ID NO:11, and SEQ ID NO:12;
e) a primer probe combination having the nucleic acid sequences of SEQ ID NO: 13, SEQ ID NO: 14, and SEQ ID NO:15;
f) a primer probe combination having the nucleic acid sequences of SEQ ID NO:20, SEQ ID NO:21, and SEQ ID NO:22;
g) a primer probe combination having the nucleic acid sequences of SEQ ID NO:23, SEQ ID NO:24, and SEQ ID NO:25; and
h) a primer probe combination having the nucleic acid sequences of SEQ ID NO:26, SEQ ID NO:27, and SEQ ID NO:28.

17. The primer probe combination according to claim 16, wherein SEQ ID NO: 3, SEQ ID NO:6, SEQ ID NO:9, SEQ ID NO:12, SEQ ID NO: 15 have a fluorescent reporter dye and/or a non-fluorescent quencher.

18. The primer probe combination according to claim 16, wherein one or more of SEQ ID NO: 3, SEQ ID NO:6, SEQ ID NO:9, SEQ ID NO:12, SEQ ID NO:15, SEQ ID NO:22, SEQ ID NO:25; or SEQ ID NO:28 have a fluorescent reporter dye 6-carboxyfluorescein (FAM) at the 5′ end and/or either a Minor Groove Binder non-fluorescence quencher (MGB-NFQ) or ZEN-IB and Iowa Black Fluorescence quencher (IBFQ) at the 3′ end.

19. The primer probe combination according to claim 15, wherein the primer probe combination detects DNA encoding the structural capsid (VP) protein and/or the non-structural (NS) protein of Bovine parvovirus 3 in a test sample.

20. An internal positive control primer probe combination for detecting Bovine parvovirus 3 (BPV-3) genomic DNA in a test sample according to claim 15, wherein the primer probe combination has the nucleic acid sequences of SEQ ID NO: 16, SEQ ID NO: 17, and SEQ ID NO: 18.

21. The internal positive control primer probe combination according to claim 20, wherein SEQ ID NO: 18 has a fluorescent reporter dye and/or a non-fluorescent quencher.

22. The primer probe combination according to claim 20, wherein SEQ ID NO: 18 has a fluorescent reporter dye, 2′-chloro-7′phenyl-1,4-dichloro-6-carboxy-fluorescein (VIC) at the 5′ end and a Minor Groove Binder non-fluorescent quencher (MGB-NFQ) at the 3′ end.

23. A primer probe combination for detecting Bovine parvovirus 3 genomic DNA in the extracted DNA of a test sample comprising SEQ ID NO:7, SEQ ID NO:8, and SEQ ID NO:9 in combination with an internal positive control primer probe combination comprising SEQ ID NO: 16, SEQ ID NO: 17, and SEQ ID NO:18, wherein SEQ ID NO: 9 has a fluorescent reporter dye 6-carboxyfluorescein at the 5′ end and a Minor Groove Binder non-fluorescent quencher (MGB-NFQ) non-fluorescence quencher at the 3′ end and SEQ ID NO: 18 has a fluorescent reporter dye, 2′-chloro-7′phenyl-1,4-dichloro-6-carboxy-fluorescein at the 5′ end and a Minor Groove Binder non-fluorescent quencher (MGB-NFQ) at the 3′ end.

24. A kit for detecting Bovine parvovirus 3 (BPV-3) genomic DNA contamination in the extracted DNA of a test sample comprising
primer probe combination that detects DNA encoding BPV-3 selected from the group consisting of
a) a primer probe combination having the nucleic acid sequences of SEQ ID NO:1, SEQ ID NO:2, and SEQ ID NO:3;
b) a primer probe combination having the nucleic acid sequences of SEQ ID NO:4, SEQ ID NO:5, and SEQ ID NO:6;
c) a primer probe combination having the nucleic acid sequences of SEQ ID NO:7, SEQ ID NO:8, and SEQ ID NO:9;
d) a primer probe combination having the nucleic acid sequences of SEQ ID NO:10, SEQ ID NO:11, and SEQ ID NO:12;
e) a primer probe combination having the nucleic acid sequences of SEQ ID NO: 13, SEQ ID NO: 14, and SEQ ID NO:15;
f) a primer probe combination having the nucleic acid sequences of SEQ ID NO:20, SEQ ID NO:21, and SEQ ID NO:22;
g) a primer probe combination having the nucleic acid sequences of SEQ ID NO:23, SEQ ID NO:24, and SEQ ID NO:25; and
h) a primer probe combination having the nucleic acid sequences of SEQ ID NO:26, SEQ ID NO:27, and SEQ ID NO:28; and
optionally an internal positive control primer probe combination having the nucleic acid sequences of SEQ ID NO: 16, SEQ ID NO:17, and SEQ ID NO:18.

25. A method for determining the presence or absence of Bovine parvovirus 3 genomic DNA in the extracted DNA of a test sample comprising
1) a reaction mixture comprising a test sample, a positive control, a BPV-3_IPC positive control plasmid DNA, nucleic acid amplification reagents, an internal positive control primer probe combination having the nucleic acid sequences of SEQ ID NO: 16, SEQ ID NO: 17, and SEQ ID NO: 18, wherein SEQ ID NO: 18 has a fluorescent reporter dye at the 5′ end and a non-fluorescence quencher at the 3′ end, and a primer probe combination selective for a DNA sequence of Bovine parvovirus 3, selected from the group consisting of
a) a primer probe combination having the nucleic acid sequences of SEQ ID NO:1, SEQ ID NO:2, and SEQ ID NO:3, wherein SEQ ID NO:3 has a fluorescent reporter dye at the 5′ end and a non-fluorescence quencher at the 3′ end;
b) a primer probe combination having the nucleic acid sequences of SEQ ID NO:4, SEQ ID NO:5, and SEQ ID NO:6, wherein SEQ ID NO:6 has a fluorescent reporter dye at the 5′ end and a non-fluorescence quencher at the 3′ end;
c) a primer probe combination having the nucleic acid sequences of SEQ ID NO:7, SEQ ID NO:8, and SEQ ID NO:9, wherein SEQ ID NO:9 has a fluorescent reporter dye at the 5′ end and a non-fluorescence quencher at the 3′ end;
d) a primer probe combination having the nucleic acid sequences of SEQ ID NO:10, SEQ ID NO:11, and SEQ ID NO: 12, wherein SEQ ID NO: 12 has a fluorescent reporter dye at the 5′ end and a non-fluorescence quencher at the 3′ end;
e) a primer probe combination having the nucleic acid sequences of SEQ ID NO: 13, SEQ ID NO: 14, and SEQ ID NO: 15, wherein SEQ ID NO: 15 has a fluorescent reporter dye at the 5′ end and a non-fluorescence quencher at the 3′ end;
f) a primer probe combination having the nucleic acid sequences of SEQ ID NO:20, SEQ ID NO:21, and SEQ ID NO:22, wherein SEQ ID NO:22 has a fluorescent reporter dye at the 5′ end and a non-fluorescence quencher at the 3′ end;
g) a primer probe combination having the nucleic acid sequences of SEQ ID NO:23, SEQ ID NO:24, and SEQ ID NO:25, wherein SEQ ID NO:25 has a fluorescent reporter dye at the 5′ end and a non-fluorescence quencher at the 3′ end; and
h) a primer probe combination having the nucleic acid sequences of SEQ ID NO:26, SEQ ID NO:27, and SEQ ID NO:28, wherein SEQ ID NO:28 has a fluorescent reporter dye at the 5′ end and a non-fluorescence quencher at the 3′ end;
2) subjecting the reaction mixture to a quantitative PCR technique to obtain copies of the target sequence,
3) measuring any increase in fluorescence signal,
wherein an increase in fluorescence signal indicates the presence of Bovine parvovirus 3 genomic DNA in the test sample.

26. The method according to claim 25, wherein the fluorescent reporter dye is 6-carboxyfluorescein (FAM) and the non-fluorescence quencher is a Minor Groove Binder non-fluorescent quencher (MGB-NFQ) or ZEN-IB and Iowa Black Fluorescence quencher (IBFQ).

27. The method according to claim 25, further comprising one or more negative extraction control, no template control, positive extraction control, positive control, and/or inhibition control.

28. The method according to claim 25, wherein the sensitivity or analytical limit of detection is 22 genome copies per reaction.

29. The method according to claim 25, wherein the sample limit of detection is 25 genome copies per reaction.

30. The method according to claim 25, wherein the primer probe combination selective for a DNA sequence of Bovine parvovirus 3 detects DNA encoding the non-structural (NS) protein and/or structural capsid (VP) protein of Bovine parvovirus 3.

31. The method according to claim 25, wherein the primer probe combination selective for a DNA sequence of Bovine parvovirus 3 amplifies a 144 bp fragment.

32. The method according to claim 25, wherein the primer probe combination selective for a DNA sequence of Bovine parvovirus 3 comprises the combination of SEQ ID NO:7, SEQ ID NO:8, and SEQ ID NO:9, wherein SEQ ID NO:9 has a 6-carboxyfluorescein (FAM) at the 5′ end and a Minor Groove Binder (MGB-NFQ) at the 3′ end.

33. The method according to claim 25 further comprising an internal positive control primer probe combination.

34. The method according to claim 33, wherein the primer probe combination has the nucleic acid sequences of SEQ ID NO: 16, SEQ ID NO: 17, and SEQ ID NO: 18.

35. The method according to claim 34, wherein SEQ ID NO: 18 has a fluorescent reporter dye and/or a non-fluorescent quencher.

36. The method according to claim 34, wherein SEQ ID NO: 18 has a fluorescent reporter dye, 2′-chloro-7′phenyl-1,4-dichloro-6-carboxy-fluorescein (VIC) at the 5′ end and a Minor Groove Binder non-fluorescent quencher (MGB-NFQ) at the 3′ end.

37. A method for the quantification of 1E3 to 1E8 genome copies of Bovine parvovirus 3 genomic DNA in a PCR reaction comprising
1) a reaction mixture comprising the extracted DNA of a test sample, a positive control, a BPV-3_IPC positive control plasmid DNA, nucleic acid amplification reagents, an internal positive control primer probe combination having the nucleic acid sequences of SEQ ID NO: 16, SEQ ID NO: 17, and SEQ ID NO: 18, wherein SEQ ID NO: 18 has a fluorescent reporter dye at the 5′ end and a non-fluorescence quencher at the 3′ end, and a primer probe combination selective for a DNA sequence of Bovine parvovirus 3, selected from the group consisting of
a) a primer probe combination having the nucleic acid sequences of SEQ ID NO:1, SEQ ID NO:2, and SEQ ID NO:3, wherein SEQ ID NO:3 has a fluorescent reporter dye at the 5′ end and a non-fluorescence quencher at the 3′ end;
b) a primer probe combination having the nucleic acid sequences of SEQ ID NO:4, SEQ ID NO:5, and SEQ ID NO:6, wherein SEQ ID NO:6 has a fluorescent reporter dye at the 5′ end and a non-fluorescence quencher at the 3′ end;
c) a primer probe combination having the nucleic acid sequences of SEQ ID NO:7, SEQ ID NO:8, and SEQ ID NO:9, wherein SEQ ID NO:9 has a fluorescent reporter dye at the 5′ end and a non-fluorescence quencher at the 3′ end;
d) a primer probe combination having the nucleic acid sequences of SEQ ID NO:10, SEQ ID NO:11, and SEQ ID NO: 12, wherein SEQ ID NO: 12 has a fluorescent reporter dye at the 5′ end and a non-fluorescence quencher at the 3′ end;
e) a primer probe combination having the nucleic acid sequences of SEQ ID NO: 13, SEQ ID NO: 14, and SEQ ID NO: 15, wherein SEQ ID NO: 15 has a fluorescent reporter dye at the 5′ end and a non-fluorescence quencher at the 3′ end; f) a primer probe combination having the nucleic acid sequences of SEQ ID NO:20, SEQ ID NO:21, and SEQ ID NO:22, wherein SEQ ID NO:22 has a fluorescent reporter dye at the 5′ end and a non-fluorescence quencher at the 3′ end;
g) a primer probe combination having the nucleic acid sequences of SEQ ID NO:23, SEQ ID NO:24, and SEQ ID NO:25, wherein SEQ ID NO:25 has a fluorescent reporter dye at the 5′ end and a non-fluorescence quencher at the 3′ end; and
h) a primer probe combination having the nucleic acid sequences of SEQ ID NO:26, SEQ ID NO:27, and SEQ ID NO:28, wherein SEQ ID NO:28 has a fluorescent reporter dye at the 5′ end and a non-fluorescence quencher at the 3′ end;
2) subjecting the reaction mixture to a quantitative PCR technique to obtain copies of the target sequence, and
3) measuring any increase in fluorescence signal.

38. The method according to claim 37, wherein the limit of detection (LOD95%) of the method is 27 genome copies of Bovine parvovirus 3 genomic DNA per reaction with a 95% confidence interval of 22 and 34 genome copies per reaction.

39. The method according to claim 37, wherein the linearity of the method has a correlation coefficient (R2) ≥0.98 and a PCR amplification efficiency within 90-110%.

40. The method according to claim 37, wherein the method has a repeatability value that is a % CV of quantity equal or less than 25%.

41. The method according to claim 37, wherein the method has an intermediate precision value that is % CV of quantity equal or less than 30%.

42. The method according to claim 37, wherein the method has an accuracy value within ±30% of the accepted reference value (ST) across the whole dynamic range of the assay.

43. The method according to claim 37, wherein the method has a limit of quantitation that is the % CV of quantity for repeatability at ≤25%, intermediate precision at ≤30% and acceptance criterion for the accuracy within ±30% of the expected standard reference value.

44. The method according to claim 37, wherein the method has a robustness that has a percent CV of quantity for repeatability of ≤25%, an intermediate precision of ≤30%, and an accuracy of the mean of quantity of the combination matrix condition tested of ±30% of the mean of quantity of the optimized condition.

45. The method according to claim 37, wherein the method includes one or more of a no template control, a positive control, a negative extraction control, a positive extraction control, an inhibition control, an internal positive control, and a standard.
